(19) United States
(12) Patent Application Publication
(10) Pub. No.: US 2019/0389850 A1
(43) Pub. Date: Dec. 26, 2019

(54) COMPOUNDS

(71) Applicant: GLAXOSMITHKLINE INTELLECTUAL PROPERTY DEVELOPMENT LIMITED, Brentford (GB)

(72) Inventors: Feng REN, Shanghai (CN); Yingxia SANG, Shanghai (CN); Weiqiang XING, Shanghai (CN); Yang ZHAN, Shanghai (CN); Baowei ZHAO, Shanghai (CN)

(21) Appl. No.: 16/480,937

(22) PCT Filed: Jan. 23, 2018
(86) PCT No.: PCT/CN2018/073846

§ 371 Date: Jul. 25, 2019

(30) Foreign Application Priority Data
Jan. 25, 2017 (CN) PCT/CN2017/072587
Jan. 25, 2017 (CN) PCT/CN2017/072610
Jan. 25, 2017 (CN) PCT/CN2017/072612

(51) Int. Cl. C07D 413/14

(52) U.S. Cl. CPC C07D 413/14

(57) ABSTRACT

The present invention provides novel compounds that inhibit LRRK2 kinase activity, processes for their preparation, compositions containing them and their use in the treatment of or prevention of diseases associated with or characterized by LRRK2 kinase activity, for example Parkinson's disease, Alzheimer's disease and amyotrophic lateral sclerosis (ALS).

### FIELD OF THE INVENTION

The present invention relates to novel compounds that inhibit LRRK2 kinase activity, processes for their preparation, compositions containing them and their use in the treatment of diseases associated with or characterized by LRRK2 kinase activity, for example, Parkinson's disease, Alzheimer's disease and amyotrophic lateral sclerosis (ALS).

### BACKGROUND OF THE INVENTION

Parkinson's disease (PD) is a neurodegenerative disorder characterized by selective degeneration and cell death of dopaminergic neurons in the substantial nigra region of the brain. Parkinson's disease was generally considered to be sporadic and of unknown etiology, but, in the last 15 years, there has been an important development of the understanding of the genetic basis of this disease and associated pathogenic mechanisms. One area of the development is the understanding of leucine rich repeat kinase 2 (LRRK2) protein. A number of mis-sense mutations in the LRRK2 gene have been strongly linked with autosomal dominant Parkinson's disease in familial studies (See WO2006068492 and WO2006045392; Trinh and Farrer 2013, Nature Reviews in Neurology 9: 445-454; Paisan-Ruiz et al., 2013, J. Parkinson's Disease 3: 85-103). The G2019S mutation in LRRK2 is the most frequent mis-sense mutation and is associated with a clinical phenotype that closely resembles sporadic Parkinson's disease. The LRRK2 G2019S mutation is also present in approximately 1.5% of sporadic Parkinson's disease cases (See Gilks et al., 2005, Lancet, 365: 415-416). In addition to the known pathogenic coding mutations in LRRK2, additional amino acid coding variants of LRRK2 have been identified that are also associated with risk of developing Parkinson's disease (See Ross et al., 2011 Lancet Neurology 10: 898-908). Furthermore, genome-wide association studies (GWAS) have identified LRRK2 as a Parkinson's disease susceptibility locus, which indicates that LRRK2 may be also relevant to sporadic Parkinson's disease cases without mutations that cause amino acid substitutions in the LRRK2 protein. (See Satake et al., 2009 Nature Genetics 41:1303-1307; Simon-Sanchez et al 2009 Nature Genetics 41: 1308-1312)

LRRK2 is a member of the ROCO protein family and all members of this family share five conserved domains. The most common pathogenic mutation G2019S occurs in the highly conserved kinase domain of LRRK2. This mutation confers an increase in the LRRK2 kinase activity in in vitro enzyme assays of recombinant LRRK2 proteins (See Jaleel et al., 2007, Biochem J, 405: 307-317) and in LRRK2 proteins purified from G2019S PD patient-derived cells (See Dzamko et al., 2010 Biochem. J. 430: 405-413). A less frequent LRRK2 pathogenic mutation that confers amino acid substitution at a different residue, R1441, has also been shown to elevate LRRK2 kinase activity by decreasing the rate of GTP hydrolysis by the GTPase domain of LRRK2 (See Guo et al., 2007 Exp Cell Res. 313: 3658-3670; West et al., 2007 Hum. Mol Gen. 16: 223-232). Moreover, phosphorylation of Rab protein physiologic substrates of LRRK2 has been shown to be increased by a range of Parkinson's disease pathogenic mutations of LRRK2 (See Steger et al., 2016 eLife 5 e12813). Therefore, the evidence indicates that the kinase and GTPase activities of LRRK2 are important for pathogenesis, and that the LRRK2 kinase domain may regulate overall LRRK2 function (See Cookson, 2010 Nat. Rev. Neurosci. 11: 791-797).

There is evidence to show that the increased LRRK2 kinase activity is associated with neuronal toxicity in cell culture models (See Smith et al., 2006 Nature Neuroscience 9: 1231-1233) and kinase inhibitor compounds protect against LRRK2-mediated cell death (See Lee et al., 2010 Nat. Med. 16: 998-1000). LRRK2 has also been reported to act as a negative regulator of microglial-mediated clearance of alpha-synuclein (See Maekawa et al., 2016 BMC Neuroscience 17:77), suggesting a possible utility of LRRK2 inhibitors in promoting clearance of neurotoxic forms of alpha-synuclein in the treatment of Parkinson's disease.

Induced pluripotent stem cells (iPSCs) derived from LRRK2 G2019S Parkinson's disease patients have been found to exhibit defects in neurite outgrowth and increased susceptibility to rotenone, that may be ameliorated by either genetic correction of the G2019S mutation or treatment of cells with small molecule inhibitors of LRRK2 kinase activity (See Reinhardt et al., 2013 Cell Stem Cell 12: 354-367). Mitochondrial DNA damage has been reported as a molecular marker of vulnerable dopamine neurons in substantia nigra of postmortem Parkinson's disease specimens (See Sanders et al 2014 Neurobiol. Dis. 70: 214-223). Increased levels of such mitochondrial DNA damage associated with LRRK2 G2019S mutation in iSPCs is blocked by genetic correction of the G2019S mutation (See Sanders et al., 2014 Neurobiol. Dis. 62: 381-386).

Additional evidence links LRRK2 function and dysfunction with autophagy-lysosomal pathways (See Manzoni and Lewis, 2013 Faseb J. 27:3234-3429). LRRK2 proteins confer defects in chaperone-mediated autophagy that negatively impact the ability of cells to degrade alpha-synuclein (Orenstein et al., 2013 Nature Neurosci. 16 394-406). In other cell models, selective LRRK2 inhibitors have been shown to stimulate macroautophagy (See Manzoni et al., 2013 BBA Mol. Cell Res. 1833: 2900-2910). These data suggest that small molecule inhibitors of LRRK2 kinase activity may have utility in the treatment of diseases characterized by defects in cellular proteostasis that result from aberrant autophagy/lysosomal degradation pathways including forms of Parkinson's disease associated with GBA mutations (See Swan and Saunders-Pullman 2013 Curr. Neurol. Neurosci Rep. 13: 368), other alpha-synucleinopathies, tauopathies, Alzheimer's disease (See Li et al., 2010 Neurodegen. Dis. 7: 265-271) and other neurodegenerative diseases (See Nixon 2013 Nat. Med. 19: 983-997) and Gaucher disease (See Westbroek et al., 2011 Trends. Mol. Med. 17: 485-493). As promoters of autophagy, small molecule inhibitors of LRRK2 kinase may also have utility in treatment of other diseases including diabetes, obesity, motor neuron disease, epilepsy and some cancers (See Rubinsztein et al., 2012 Nat. Rev. Drug Discovery 11: 709-730), pulmonary diseases such as chronic obstructive pulmonary disease and idiopathic pulmonary fibrosis (See Araya et al., 2013 Intern. Med. 52: 2295-2303) and autoimmune diseases such as systemic lupus erythematosus (See Martinez et al., 2016 Nature 533: 115-119). As promoters of autophagy and phagocytic processes, small molecule inhibitors of LRRK2 kinase may also have utility in augmenting host responses in treatment of a range of intracellular bacterial infections, parasitic infections and viral infections, including diseases such as tuberculosis (See Rubinsztein et al., 2012 Nat. Rev. Drug Discovery 11: 709-730; Araya et al., 2013 Intern. Med. 52: 2295-2303; Gutierrez, Biochemical Society Conference; Leucine rich repeat kinase 2: ten years along the road to therapeutic intervention, Henley Business School, UK 12 Jul. 2016), HIV, West Nile Virus and chikungunya virus (see Shoji-Kawata et al., 2013 Nature 494: 201-206). LRRK2 inhibitors may have utility in treatment of such diseases alone, or in combination with drugs that directly target the infectious agent. Further, significantly elevated levels of LRRK2 mRNA have also been observed in fibroblasts of Niemann-Pick Type C (NPC) disease patients compared with fibroblasts of normal subjects, which indicates that aberrant LRRK2 function may play a role in lysosomal disorders (See Reddy et al., 2006 PLOS One 1 (1):e19 doi: 10.1371/journal.pone.0000019—supporting information Dataset S1). This observation suggests that LRRK2 inhibitors may have utility for treatment of NPC.

The PD-associated G2019S mutant form of LRRK2 has also been reported to enhance phosphorylation of tubulin-associated Tau (See Kawakami et al., 2012 PLoS ONE 7: e30834, doi 10.1371), and disease models have been proposed in which LRRK2 acts upstream of the pathogenic effects of Tau and alpha-synuclein (See Taymans & Cookson, 2010, BioEssays 32: 227-235). In support of this, LRRK2 expression has been associated with increased aggregation of insoluble Tau, and increased Tau phosphorylation, in a transgenic mouse model (See Bailey et al., 2013 Acta Neuropath. 126:809-827). Over-expression of the PD pathogenic mutant protein LRRK2R1441G is reported to cause symptoms of Parkinson's disease and hyperphosphorylation of Tau in transgenic mouse models (See Li, Y. et al. 2009, Nature Neuroscience 12: 826-828). Therefore, these data suggest that LRRK2 inhibitors of kinase catalytic activity may be useful for the treatment of tauopathy diseases characterized by hyperphosphorylation of Tau such as argyrophilic grain disease, Pick's disease, corticobasal degeneration, progressive supranuclear palsy and inherited frontotemporal dementia and parkinsonism linked to chromosome 17 (FTDP-17) (See Goedert, M and Jakes, R (2005) Biochemica et Biophysica Acta 1739, 240-250). In addition, LRRK2 inhibitors may have utility in treatment of other diseases characterized by diminished dopamine levels such as withdrawal symptoms/relapse associated with drug addiction (See Rothman et al., 2008, Prog. Brain Res, 172: 385).

Other studies have also shown that overexpression of the G2019S mutant form of LRRK2 confers defects in subventricular zone (SVZ) neuroprogenitor cell proliferation and migration in transgenic mouse models (See Winner et al., 2011 Neurobiol. Dis. 41: 706-716) and reduces neurite length and branching cell culture models (See Dachsel et al., 2010 Parkinsonism & Related Disorders 16: 650-655). Moreover, it was reported that agents that promote SVZ neuroprogenitor cell proliferation and migration also improve neurological outcomes following ischemic injury in rodent models of stroke (See Zhang et al., 2010 J. Neurosci. Res. 88: 3275-3281). These findings suggest that compounds that inhibit aberrant activity of LRRK2 may have utility for the treatments designed to stimulate restoration of CNS functions following neuronal injury, such as ischemic stroke, traumatic brain injury, spinal cord injury.

Mutations in LRRK2 have also been identified that are clinically associated with the transition from mild cognitive impairment (MCI) to Alzheimer's disease (See WO2007149798). These data suggest that inhibitors of LRRK2 kinase activity may be useful for the treatment diseases such as Alzheimer's disease, other dementias and related neurodegenerative disorders.

Aberrant regulation of normal LRRK2 proteins is also observed in some disease tissues and models of disease. Normal mechanisms of translational control of LRRK2 by miR-205 are perturbed in some sporadic PD cases, where significant decreases in miR-205 levels in PD brain samples concur with elevated LRRK2 protein levels in those samples (See Cho et al., (2013) Hum. Mol. Gen. 22: 608-620). Therefore, LRRK2 inhibitors may be used in treatment of sporadic PD patients who have elevated levels of normal LRRK2 proteins.

In an experimental model of Parkinson's disease in marmosets, an elevation of LRRK2 mRNA is observed in a manner that correlates with the level of L-Dopa induced dyskinesia (See Hurley, M. J et al., 2007 Eur. J. Neurosci. 26: 171-177). This suggests that LRRK2 inhibitors may have a utility in amelioration of such dyskinesias.

Significantly elevated levels of LRRK2 mRNA have been reported in ALS patient muscle biopsy samples (See Shtilbans et al., 2011 Amyotrophic Lateral Sclerosis 12: 250-256) It is suggested that elevated levels of LRRK2 kinase activity may be a characteristic feature of ALS. Therefore, this observation indicated that LRRK2 inhibitor may have utility for treatment of ALS.

There is also evidence indicating that LRRK2 kinase activity may play a role in mediating microglial proinflammatory responses (See Moehle et al., 2012, J. Neuroscience 32: 1602-1611). This observation suggests a possible utility of LRRK2 inhibitors for treatment of aberrant neuroinflammatory mechanisms that contribute a range of neurodegenerative diseases, including Parkinson's disease, Alzheimer's disease, multiple sclerosis, HIV-induced dementia, amyotrophic lateral sclerosis, ischemic stroke, traumatic brain injury and spinal cord injury. Some evidence also indicates that LRRK2 plays a role in regulating neuronal progenitor differentiation in vitro (See Milosevic, J. et al., 2009 Mol. Neurodegen. 4: 25). This evidence suggests that inhibitors of LRRK2 may have a utility in production of neuronal progenitor cells in vitro for consequent therapeutic application in cell based-treatment of CNS disorders.

It has been reported that Parkinson's disease patients bearing LRRK2 G2019S mutation display increased frequency of non-skin cancers, including renal, breast, lung, prostate cancers as well as acute myelogenous leukemia (AML). Since there is evidence to show that G2019S mutation in LRRK2 increases catalytic activity of the LRRK2 kinase domain, small molecule inhibitors of LRRK2 may have a utility in treatment of cancers, for example kidney cancer, breast cancer, lung cancer, prostate cancer (e.g. solid tumors) and blood cancer (See. AML; Saunders-Pullman et al., 2010, Movement Disorders, 25:2536-2541; Inzelberg et al., 2012 Neurology 78: 781-786). Amplification and over-expression of LRRK2 has also been reported in papillary renal and thyroid carcinomas, where co-operativity between LRRK2 and the MET oncogene may promote tumor cell growth and survival (See Looyenga et al., 2011 PNAS 108: 1439-1444.)

Some studies suggested that genetic association of common LRRK2 variants with susceptibility to ankylosing spondylitis (See Danoy P, et al., 2010. PLoS Genet.; 6(12):e1001195; and leprosy infection. (See Zhang F R, et al. 2009, N Engl J Med. 361:2609-18.) These findings suggest that inhibitors of LRRK2 may have a utility in the treatment of ankylosing spondylitis and leprosy infection.

Meta-analysis of three genome wide associated scans for Crohn's disease identified a number of loci associated with the disease, including the locus containing the LRRK2 gene (See Barrett et al., 2008, Nature Genetics, 40: 955-962). Evidence has also emerged that LRRK2 is an IFN-γ target gene that may be involved in signaling pathways relevant to Crohn's disease pathogenesis (See Gardet et al., 2010, J. Immunology, 185: 5577-5585). These findings suggest that inhibitors of LRRK2 may have utility in the treatment of Crohn's disease.

As an IFN-γ target gene, LRRK2 may also play a role in T cell mechanisms that underlie other diseases of the immune system such as multiple sclerosis and rheumatoid arthritis. Further potential utility of LRRK2 inhibitors comes from the reported finding that B lymphocytes constitute a major population of LRRK2 expressing cells (See Maekawa et al. 2010, BBRC 392: 431-435). This suggests that LRRK2 inhibitors may be effective in treatment of diseases of the immune system for which B cell depletion is, or may be, effective in diseases such as lymphomas, leukemias, multiple sclerosis (See Ray et al., 2011 J. Immunol. 230: 109), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, Sjogren's syndrome, Devic's disease and inflammatory myopathies (See Engel et al., 2011 Pharmacol. Rev. 63: 127-156; Homam et al., 2010 J. Clin. Neuromuscular Disease 12: 91-102).

WO2016036586 and WO2017012576 disclose a series of compounds described as inhibitors of LRRK2 kinase and their use in the treatment of diseases, including, inter alia, Parkinson's disease. Unmet needs exist for new treatments that will halt or slow disease progression both in terms of motor (e.g. control of gait dysfunction, freezing, and postural imbalance) and non-motor symptoms (e.g. PD-associated dementia), reducing the need for escalating use of symptomatic medications and associated long-term adverse effects of currently available treatment (e.g. dyskinesia and on/off fluctuations) maintaining independence for longer.

### SUMMARY OF THE INVENTION

The present invention provides, in a first aspect, compounds of Formula (I) and salts thereof:

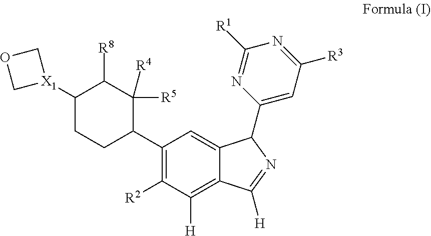

wherein
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃haloalkyl;
R³ is selected from the group consisting of:
a) an N-linked 4-6 membered heterocyclyl ring optionally substituted with one, two, three or four substituents independently selected from the group consisting of:
   halo,
   hydroxyl,
   C₁₋₆alkyl, optionally substituted with one or two substituents independently selected from the group consisting of: halo, hydroxyl, C₁₋₃alkoxy and cyclopropyl, and
   C₁₋₆ alkoxyl, which alkoxyl group is optionally substituted with one or two substituents independently selected from the group consisting of halo, hydroxyl and C₁₋₃ alkoxyl,
wherein when the N-linked 4-6 membered heterocyclyl ring contains a substitutable nitrogen atom, the group of substituents also includes a 4-6 membered heterocyclyl ring which is optionally substituted with one, two or three substituents independently selected from halo, hydroxyl, and C₁₋₃ alkoxyl, and is attached to said substitutable nitrogen atom;
b) NHR⁷; and
c) OR⁷;
R⁴ and R⁵ are independently selected from the group consisting of H, hydroxyl and halo;
X₁ is CR⁶ wherein R⁶ is C₁₋₃alkyl, which alkyl group is optionally substituted with one or two substituents independently selected from the group consisting of hydroxyl, halo and C₁₋₃alkoxyl;
R⁷ is independently selected from the group consisting of:
   C₄₋₆ cycloalkyl, which cycloalkyl is optionally substituted with one, two or three substituents independently selected from halo, hydroxyl, C₁₋₃ alkoxyl and C₁₋₃ alkyl, which alkyl group is optionally substituted with one two or three halo or hydroxyl groups, and
   a nitrogen- or oxygen-containing 4-6 membered heterocyclyl optionally substituted with one or more substituents independently selected from halo, hydroxyl, C₁₋₃ alkoxyl and C₁₋₃ alkyl, which alkyl group is optionally substituted with one, two or three halo or hydroxyl groups; and
R⁸ is hydrogen or C₁₋₃alkyl.

In a further aspect of the invention, the invention provides a pharmaceutical composition comprising a compound of Formula (I) or a pharmaceutically acceptable salt thereof and a pharmaceutically acceptable carrier.

A further aspect of the invention provides a compound of Formula (I) or a pharmaceutically acceptable salt thereof for use in the treatment or prevention of Parkinson's disease, Alzheimer's disease, or amyotrophic lateral sclerosis (ALS).

### DETAILED DESCRIPTION OF THE INVENTION

The foregoing and other aspects of the present invention will now be described in more detail with respect to the description and methodologies provided herein. It should be appreciated that the invention can be embodied in different forms and should not be construed as limited to the embodiments set forth herein. Rather, these embodiments are provided so that this disclosure will be thorough and complete, and will Fully convey the scope of the invention to those skilled in the art.

The terminology used in the description of the invention herein is for the purpose of describing particular embodiments only and is not intended to be limiting of the invention. As used in the description of the embodiments of the invention and the appended claims, the singular forms “a”, “an” and “the” are intended to include the plural forms as well, unless the context clearly indicates otherwise. Also, as used herein, “and/or” refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Generally, the nomenclature used herein and the laboratory procedures in organic chemistry, medicinal chemistry, biology described herein are those well known and commonly employed in the art. Unless defined otherwise, all technical and scientific terms used herein generally have the same meaning as commonly understood by one of ordinary skill in the art to which this disclosure belongs. In the event that there is a plurality of definitions for a term used herein, those in this section prevail unless stated otherwise.

### A. Definitions

As used herein, “alkyl” refers to a monovalent, saturated hydrocarbon chain having a specified number of carbon atoms. For example, C₁₋₃ alkyl refers to an alkyl group having from 1 to 3 carbon atoms. Alkyl groups may be straight or branched. In some embodiments, branched alkyl groups may have one, two, or three branches. Exemplary alkyl groups include, but are not limited to, methyl, ethyl, and propyl (n-propyl and isopropyl).

As used herein, “alkoxy” refers to the group —O-alkyl. For example, C₁₋₆ alkoxy groups contain from 1 to 6 carbon atoms. C₁₋₃ alkoxy groups contain from 1 to 3 carbon atoms. Exemplary alkoxy groups include, but are not limited to, methoxy, ethoxy, propoxy, butoxyl, pentyloxy, and hexyloxy.

As used herein, “cycloalkyl” refers to a saturated monocyclic hydrocarbon ring having a specified number of carbon atoms. For example, C₃₋₆ cycloalkyl contains 3 to 6 carbon atoms as member atoms in the ring. Examples of C₃₋₆ cycloalkyl include cyclopropyl, cyclobutyl, cyclopentyl and cyclohexyl.

As used herein, “halogen” refers to fluorine (F), chlorine (Cl), bromine (Br), or iodine (I). “Halo” refers to the halogen radicals: fluoro (—F), chloro (—Cl), bromo (—Br), or iodo (—I).

As used herein, “haloalkyl” refers to an alkyl group, as defined above, having one or more halogen atoms selected from F, Cl, Br, or I, which are substituted on any or all of the carbon atoms of the alkyl group by replacing hydrogen atoms attached to the carbon atoms and which may be the same or different. For example, C₁₋₃haloalkyl refers to a C₁₋₃alkyl group substituted with one or more halogen atoms. In some embodiments, “haloalkyl” refers to an alkyl group substituted with one or more halogen atoms independently selected from F or Cl. Exemplary haloalkyl groups include, but are not limited to, chloromethyl, bromoethyl, trifluoromethyl, and dichloromethyl.

As used herein, “heterocyclyl” or “herterocyclyl ring” is a monovalent radical derived by removal of a hydrogen atom from a saturated monocyclic ring, which ring consists of ring carbon atoms and 1 or more ring heteroatoms independently selected from nitrogen, oxygen or sulphur. In one embodiment, the ring consists of ring carbon atoms and 1 to 3 ring heteroatoms independently selected from nitrogen, oxygen or sulphur. In one embodiment, the ring-heteroatoms are independently selected from nitrogen or oxygen. The number of ring atoms may be specified. For example, a “4-6 membered heterocyclyl” a heterocyclyl as defined above consisting of 4-6 ring atoms. The term N-linked 4-6 membered heterocyclyl ring refers to a 4-6 membered heterocyclyl ring as defined above that contains at least one nitrogen ring atom through which it is linked to the core. Other ring heteroatoms (nitrogen, oxygen or sulphur) may additionally be present. The term nitrogen containing heterocyclyl refers to heterocyclyl ring as defined above that contains at least one nitrogen ring atom. Other ring heteroatoms (nitrogen, oxygen or sulphur) may additionally be present. The term oxygen containing heterocyclyl should be construed in an analogous manner. Examples of herterocyclyl rings include, but are not limited to, azetidinyl, tetrahydrofuranyl (including, for example, tetrahydrofuran-2-yl and tetrahydrofuran-3-yl), pyrrolidinyl (including, for example, pyrrolidin-1-yl and pyrrolidin-3-yl), piperidinyl (including, for example, piperidin-3-yl and piperidin-4-y), morpholinyl (including, for example, morpholin-2-yl and morpholin-4-yl).

As used herein, “substituted” in reference to a group indicates that one or more hydrogen atom attached to a member atom (e.g., carbon atom) within the group is replaced with a substituent selected from the group of defined substituents. It should be understood that the term “substituted” includes the implicit provision that such substitution is in accordance with the permitted valence of the substituted atom and the substituent and that the substitution results in a stable compound (i.e. one that does not spontaneously undergo transformation such as by rearrangement, cyclization, or elimination and that is sufficiently robust to survive isolation from a reaction mixture). When it is stated that a group may contain one or more substituent, one or more (as appropriate) member atom within the group may be substituted. In addition, a single member atom within the group may be substituted with more than one substituent as long as such substitution is in accordance with the permitted valence of the atom. Examples of substituted heterocyclyl rings include, but are not limited to,

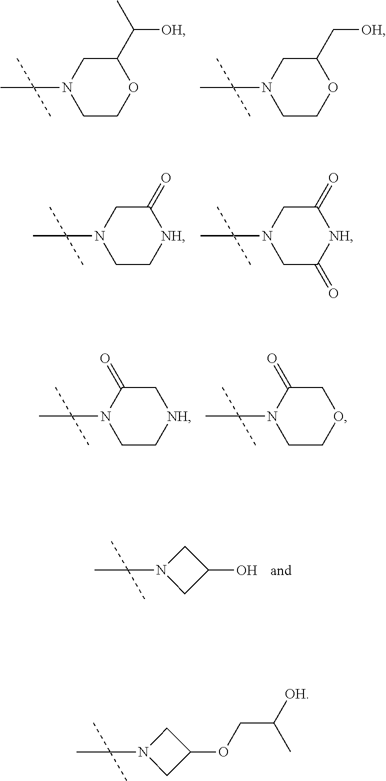

As used herein, “optionally substituted” indicates that a particular group may be unsubstituted, or may be substituted as further defined.

As used herein, the term “disease” refers to any alteration in state of the body or of some of the organs, interrupting or disturbing the performance of the functions and/or causing symptoms such as discomfort, dysfunction, distress, or even death to the person afflicted or those in contact with a person. A disease can also include a distemper, ailing, ailment, malady, disorder, sickness, illness, complain, interdisposition and/or affectation.

As used herein, “treat”, “treating” or “treatment” in reference to a disease means: (1) to ameliorate the disease or one or more of the biological manifestations of the disease, (2) to interfere with (a) one or more points in the biological cascade that leads to or is responsible for the disease or (b) one or more of the biological manifestations of the disease, (3) to alleviate one or more of the symptoms or effects associated with the disease, (4) to slow the progression of the disease or one or more of the biological manifestations of the disease, and/or (5) to diminish the likelihood of severity of a disease or biological manifestations of the disease. Symptomatic treatment refers to treatment as referred to in point (1), (3) and (5). Disease modifying treatment refers to treatment as defined in point (2) and (4).

As used herein, “prevent”, “preventing” or “prevention” means the prophylactic administration of a drug to diminish the likelihood of the onset of or to delay the onset of a disease or biological manifestation thereof.

As used herein, “subject” means a mammalian subject (e.g., dog, cat, horse, cow, sheep, goat, monkey, etc.), and human subjects including both male and female subjects, and including neonatal, infant, juvenile, adolescent, adult and geriatric subjects, and further including various races and ethnicities including, but not limited to, white, black, Asian, American Indian and Hispanic.

As used herein, “pharmaceutically acceptable salt(s)” refers to salt(s) that retain the desired biological activity of the subject compound and exhibit minimal undesired toxicological effects. These pharmaceutically acceptable salts may be prepared in situ during the final isolation and purification of the compound, or by separately reacting the purified compound in its free acid or free base form with a suitable base or acid, respectively.

As used herein, “therapeutically effective amount” in reference to a compound of the invention or other pharmaceutically-active agent means an amount of the compound sufficient to treat or prevent the patient's disease but low enough to avoid serious side effects (at a reasonable benefit/risk ratio) within the scope of sound medical judgment. A therapeutically effective amount of a compound will vary with the particular compound chosen (e.g. consider the potency, efficacy, and half-life of the compound); the route of administration chosen; the disease being treated; the severity of the disease being treated; the age, size, weight, and physical disease of the patient being treated; the medical history of the patient to be treated; the duration of the treatment; the nature of concurrent therapy; the desired therapeutic effect; and like factors, but can nevertheless be routinely determined by the skilled artisan.

### B. Compounds

This invention provides, in a first aspect, a compound of Formula (I) and salts thereof:

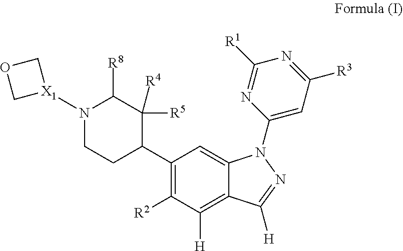

wherein
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃haloalkyl;
R³ is selected from the group consisting of:
a) an N-linked 4-6 membered heterocyclyl ring optionally substituted with one, two, three or four substituents independently selected from the group consisting of:
   halo,
   hydroxyl,
   C₁₋₆alkyl, optionally substituted with one or two substituents independently selected from the group consisting of: halo, hydroxyl, C₁₋₃alkoxy and cyclopropyl, and
   C₁₋₆ alkoxyl, which alkoxyl group is optionally substituted with one or two substituents independently selected from the group consisting of halo, hydroxyl and C₁₋₃ alkoxyl,
wherein when the N-linked 4-6 membered heterocyclyl ring contains a substitutable nitrogen atom, the group of substituents also includes a 4-6 membered heterocyclyl ring which is optionally substituted with one, two or three substituents independently selected from halo, hydroxyl, and C₁₋₃ alkoxyl, and is attached to said substitutable nitrogen atom;
b) NHR⁷; and
c) OR⁷;
R⁴ and R⁵ are independently selected from the group consisting of H, hydroxyl and halo;
X₁ is CR⁶ wherein R⁶ is C₁₋₃alkyl, which alkyl group is optionally substituted with one or two substituents independently selected from the group consisting of hydroxyl, halo and C₁₋₃alkoxyl;
R⁷ is independently selected from the group consisting of:
   C₄₋₆ cycloalkyl, which cycloalkyl is optionally substituted with one, two or three substituents independently selected from halo, hydroxyl, C₁₋₃ alkoxyl and C₁₋₃ alkyl, which alkyl group is optionally substituted with one two or three halo or hydroxyl groups, and
   a nitrogen- or oxygen-containing 4-6 membered heterocyclyl optionally substituted with one or more substituents independently selected from halo, hydroxyl, C₁₋₃ alkoxyl and C₁₋₃ alkyl, which alkyl group is optionally substituted with one, two or three halo or hydroxyl groups; and
R⁸ is hydrogen or C₁₋₃alkyl.

In a further aspect of the invention, the invention provides a pharmaceutical composition comprising a compound of Formula (I) or a pharmaceutically acceptable salt thereof and a pharmaceutically acceptable carrier.

In another aspect, the present invention provides compounds of Formula (I-A) and salts thereof:

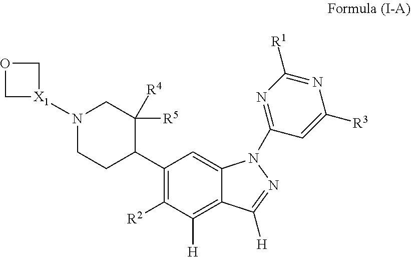

wherein
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃haloalkyl;
R³ is selected from the group consisting of:
a) an N-linked 4-6 membered heterocyclyl ring optionally substituted with one or two substituents independently selected from the group consisting of:
   halo,
   hydroxyl,
   C₁₋₆alkyl, optionally substituted with one or two substituents independently selected from the group consisting of: halo, hydroxyl, C₁₋₃alkoxy and cyclopropyl, and
   C₁₋₆ alkoxyl, which alkoxyl group is optionally substituted with one or two substituents independently selected from the group consisting of halo, hydroxyl and C₁₋₃ alkoxyl,
wherein when the N-linked 4-6 membered heterocyclyl ring contains a substitutable nitrogen atom, the group of substituents also includes a 4-6 membered heterocyclyl ring which is optionally substituted with one, two or three substituents independently selected from halo, hydroxyl, and C₁₋₃ alkoxyl, and is attached to said substitutable nitrogen atom;
b) NHR⁷; and
c) OR⁷;
R⁴ and R⁵ are independently selected from the group consisting of H, hydroxyl and halo;
X₁ is CR⁶ wherein R⁶ is C₁₋₃alkyl, which alkyl group is optionally substituted with one or two substituents independently selected from the group consisting of hydroxyl, halo and C₁₋₃alkoxyl; and
R⁷ is independently selected from the group consisting of:
   C₄₋₆ cycloalkyl, which cycloalkyl is optionally substituted with one, two or three substituents independently selected from halo, hydroxyl, C₁₋₃ alkoxyl and C₁₋₃ alkyl, which alkyl group is optionally substituted with one two or three halo or hydroxyl groups, and
   a nitrogen- or oxygen-containing 4-6 membered heterocyclyl optionally substituted with one or more substituents independently selected from halo, hydroxyl, C₁₋₃ alkoxyl and C₁₋₃ alkyl, which alkyl group is optionally substituted with one, two or three halo or hydroxyl groups.

In another aspect of the invention, the invention provides a pharmaceutical composition comprising a compound of Formula (I-A) or a pharmaceutically acceptable salt thereof and a pharmaceutically acceptable carrier.

In one embodiment, R¹ is selected from the group consisting of C₁₋₃ alkyl and C₁₋₃ alkoxyl. In one embodiment, R¹ is selected from the group consisting of methyl and methoxy. In one embodiment, R¹ is methyl.

In one embodiment, R² is selected from the group consisting of H, halo and C₁₋₃alkyl. In one embodiment, R² is C₁₋₃alkyl. In one embodiment, R² is selected from the group consisting of H, halo and methyl. In one embodiment, R² is selected from the group consisting of H, fluoro, chloro and methyl. In one embodiment, R² is selected from the group consisting of H, chloro and methyl. In one embodiment, R² is selected from the group consisting of chloro and methyl. In one embodiment, R² is methyl.

In one embodiment R³ is an N-linked 4-6 membered heterocyclyl ring optionally substituted with one or two substituents independently selected from the group consisting of:
halo, hydroxyl,
C₁ 6alkyl, which alkyl group is optionally substituted with one or two substituents independently selected from the group consisting of: halo, hydroxyl, C₁₋₃alkoxy and cyclopropyl, and
C₁₋₆ alkoxyl, which alkoxyl group is optionally substituted with one or two substituents independently selected from the group consisting of halo, hydroxyl and C₁₋₃ alkoxyl,
wherein when the N-linked 4-6 membered heterocyclyl ring contains a substitutable nitrogen atom, the group of substituents also includes a 4-6 membered heterocyclyl ring which is optionally substituted with one, two or three substituents independently selected from halo, hydroxyl, and C₁₋₃ alkoxyl, with the proviso that the 4-6 membered heterocyclyl ring is attached to said substitutable nitrogen atom.

In one embodiment R³ is an N-linked 4-6 membered heterocyclyl ring optionally substituted with one or two substituents independently selected from the group consisting of:
halo,
hydroxyl,
C₁₋₃alkyl, which alkyl group is optionally substituted with one or two substituents independently selected from the group consisting of: halo, hydroxyl and C₁₋₃alkoxy, and
C₁₋₃ alkoxyl, which alkoxyl group is optionally substituted with one or two substituents independently selected from the group consisting of halo, hydroxyl and C₁₋₃ alkoxyl.

In one embodiment R³ is an N-linked 4-6 membered heterocyclyl ring selected from the group consisting of morpholinyl, azetidinyl, pyrrolidinyl and piperazinyl, optionally substituted with one or two substituents independently selected from the group consisting of:
halo,
hydroxyl,
C₁₋₃alkyl, which alkyl group is optionally substituted with one or two substituents independently selected from the group consisting of: halo, hydroxyl and C₁₋₃alkoxy, and
C₁₋₃ alkoxyl, which alkoxyl group is optionally substituted with one or two substituents independently selected from the group consisting of halo, hydroxyl and C₁₋₃ alkoxyl.

In one embodiment R³ is an N-linked 4-6 membered heterocyclyl ring selected from the group consisting of morpholinyl, azetidinyl, pyrrolidinyl and piperazinyl, optionally substituted with one or two substituents independently selected from the group consisting of:
hydroxyl,
C₁₋₃alkyl, which alkyl group is optionally substituted with one or two substituents independently selected from the group consisting of: halo, hydroxyl and C₁₋₃alkoxy, and
C₁₋₃ alkoxyl, which alkoxyl group is optionally substituted with one or two substituents independently selected from the group consisting of halo, hydroxyl and C₁₋₃ alkoxyl.

In one embodiment R³ is an N-linked morpholinyl ring optionally substituted with one or two substituents independently selected from the group consisting of:
hydroxyl,
C₁₋₃alkyl, which alkyl group is optionally substituted with one or two substituents independently selected from the group consisting of: halo, hydroxyl and C₁₋₃ alkoxy, and
C₁₋₃ alkoxyl, which alkoxyl group is optionally substituted with one or two substituents independently selected from the group consisting of halo, hydroxyl and C₁₋₃ alkoxyl.

In one embodiment R³ is an N-linked morpholinyl ring optionally substituted with one C₁₋₃ alkyl substituent, which alkyl group is optionally substituted with one or two substituents independently selected from the group consisting of: halo, hydroxyl and C₁₋₃alkoxy.

In one embodiment, R³ is (2-hydroxymethyl)-morpholin-4-yl.

In one embodiment R³ is an N-linked 4-6 membered heterocyclyl ring containing a substitutable nitrogen atom, substituted with a further 4-6 membered heterocyclyl ring which is optionally substituted with one, two or three substituents independently selected from the group consisting of halo, hydroxyl, and C₁₋₃ alkoxyl, and with the proviso that the further 4-6 membered heterocyclyl ring is attached to said substitutable nitrogen atom.

In one embodiment R³ is an N-linked 4-6 membered heterocyclyl ring containing a substitutable nitrogen atom, substituted with an oxetanyl group on said substitutable nitrogen atom.

In one embodiment, R⁴ and R⁵ are independently selected from the group consisting of H and halo. In one embodiment, R⁴ and R⁵ are independently selected from the group consisting of H and fluoro. In one embodiment, R⁴ and R⁵ are both hydrogen.

In one embodiment, R⁶ is unsubstituted C₁₋₃alkyl. In one embodiment, R⁶ is methyl.

In one embodiment, the invention provides a compound of Formula (I) or a salt thereof wherein R¹, R², R⁴, R⁵, X₁ and R⁶ are as defined above, and R³ is an N-linked 4-6 membered heterocyclyl ring optionally substituted with one or two substituents independently selected from the group consisting of: halo, hydroxyl, C₁₋₃alkyl (which alkyl group is optionally substituted with one or two substituents independently selected from the group consisting of: halo, hydroxyl and C₁₋₃alkoxyl) and C₁₋₃ alkoxyl (which alkoxyl group is optionally substituted with one or two substituents independently selected from halo, hydroxyl and C₁₋₃ alkoxyl). In this embodiment, R¹, R², R⁴, R⁵, X₁ and R⁶ may be further defined as in any of the preceding embodiments. For example, R¹ may be selected from the group consisting of C₁₋₃ alkyl and C₁₋₃ alkoxyl and R² may be selected from the group consisting of H, halo and C₁₋₃alkyl.

In one embodiment, the compound of Formula (I) or a pharmaceutically acceptable salt thereof is a compound of any one of Examples 1 to 4, or a salt thereof.

In one embodiment, the compound of formula (I) or a salt thereof is (4-(2-methyl-6-(5-methyl-6-(1-(3-methyloxetan-3-yl)piperidin-4-yl)-1H-indazol-1-yl)pyrimidin-4-yl)morpholin-2-yl)methanol or)-(4-(2-methoxy-6-(5-methyl-6-(1-(3-methyloxetan-3-yl)piperidin-4-yl)-1H-indazol-1-yl)pyrimidin-4-yl)morpholin-2-yl)methanol or a salt of either of these compounds.

In one embodiment, the compound of formula (I) is (4-(2-methyl-6-(5-methyl-6-(1-(3-methyloxetan-3-yl)piperidin-4-yl)-1H-indazol-1-yl)pyrimidin-4-yl)morpholin-2-yl)methanol or (4-(2-methoxy-6-(5-methyl-6-(1-(3-methyloxetan-3-yl)piperidin-4-yl)-1H-indazol-1-yl)pyrimidin-4-yl)morpholin-2-yl)methanol.

In one embodiment, the compound of formula (I) or a pharmaceutically acceptable salt thereof is (R)-(4-(2-methyl-6-(5-methyl-6-(1-(3-methyloxetan-3-yl)piperidin-4-yl)-1H-indazol-1-yl)pyrimidin-4-yl)morpholin-2-yl)methanol or a salt thereof. In one embodiment, the compound of formula (I) is (R)-(4-(2-methyl-6-(5-methyl-6-(1-(3-methyloxetan-3-yl)piperidin-4-yl)-1H-indazol-1-yl)pyrimidin-4-yl)morpholin-2-yl)methanol.

In one embodiment, the compound of formula (I) or a pharmaceutically acceptable salt thereof is (S)-(4-(2-methoxy-6-(5-methyl-6-(1-(3-methyloxetan-3-yl)piperidin-4-yl)-1H-indazol-1-yl)pyrimidin-4-yl)morpholin-2-yl)methanol or a salt thereof. In one embodiment, the compound of formula (I) is (S)-(4-(2-methoxy-6-(5-methyl-6-(1-(3-methyloxetan-3-yl)piperidin-4-yl)-1H-indazol-1-yl)pyrimidin-4-yl)morpholin-2-yl)methanol.

In one embodiment, the invention provides a compound of Formula (I-A) or a pharmaceutically acceptable salt thereof which is a compound of any one of Examples A-1 to A-4 or a pharmaceutically acceptable salt thereof. In one embodiment, the invention provides a compound of Formula (I-A) which is a compound of any one of Examples A-1 to A-4.

The present invention provides, in a further aspect, compounds of Formula (I-B) and salts thereof:

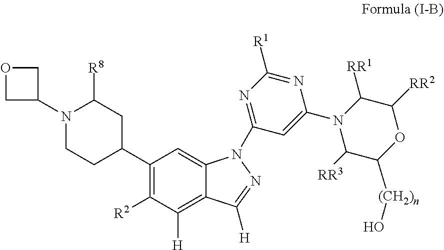

wherein
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃haloalkyl; RR¹, RR² and RR³ are independently hydrogen or C₁₋₃alkyl;
R⁸ is hydrogen or C₁₋₃alkyl; and
n is 1 or 2;
with the proviso that when n is 1 and R⁸ is hydrogen, RR², RR¹ and RR³ are not all hydrogen.

In another aspect of the invention, the invention provides a pharmaceutical composition comprising a compound of Formula (I-B) or a pharmaceutically acceptable salt thereof and a pharmaceutically acceptable carrier.

In one embodiment, R¹ is selected from the group consisting of C₁₋₃ alkyl and C₁₋₃ alkoxy. In one embodiment, R¹ is selected from the group consisting of methyl or methoxy. In one embodiment, R¹ is methyl.

In one embodiment, R² is selected from the group consisting of H, halo and C₁₋₃alkyl. In one embodiment, R² is C₁₋₃alkyl. In one embodiment, R² is selected from the group consisting of H, halo and methyl. In one embodiment, R² is selected from the group consisting of H, fluoro, chloro and methyl. In one embodiment, R² is selected from the group consisting of H, chloro and methyl. In one embodiment, R² is selected from the group consisting of chloro and methyl. In one embodiment, R² is methyl.

In one embodiment, n is 1 or 2, RR¹ is methyl, RR² is hydrogen, and RR³ is hydrogen. In one embodiment, n is 1, RR¹ is methyl, RR² is hydrogen, and RR³ is hydrogen.

In one embodiment, n is 1 or 2, RR¹ is hydrogen, RR² is methyl, and RR³ is hydrogen. In one embodiment, n is 1, RR¹ is hydrogen, RR² is methyl, and RR³ is hydrogen.

In one embodiment, n is 1 or 2, RR¹ is hydrogen, RR² is hydrogen, and RR³ is methyl. In one embodiment, n is 1, RR¹ is hydrogen, RR² is hydrogen, and RR³ is methyl.

In one embodiment, n is 2 and RR¹, RR² and RR³ are hydrogen.

In one embodiment, R⁸ is hydrogen or methyl. In one embodiment, R⁸ is hydrogen.

In one embodiment, the invention provides a compound of Formula (I-B) or a pharmaceutically acceptable salt thereof wherein n is 1. In this embodiment, R¹, R², RR², RR¹, RR³ and R⁸ may be further defined as in any of the preceding embodiments. For example, R² may be methyl.

In one embodiment, the invention provides a compound of Formula (I-B) selected from:
(6-Methyl-4-(2-methyl-6-(5-methyl-6-(1-(oxetan-3-yl)piperidin-4-yl)-1H-indazol-1-yl)pyrimidin-4-yl)morpholin-2-yl)methanol;
(5-methyl-4-(2-methyl-6-(5-methyl-6-(1-(oxetan-3-yl)piperidin-4-yl)-1H-indazol-1-yl)pyrimidin-4-yl)morpholin-2-yl)methanol;
2-(4-(2-methyl-6-(5-methyl-6-(1-(oxetan-3-yl)piperidin-4-yl)-1H-indazol-1-yl)pyrimidin-4-yl)morpholin-2-yl)ethanol;
(4-(2-methoxy-6-(5-methyl-6-(1-(oxetan-3-yl)piperidin-4-yl)-1H-indazol-1-yl)pyrimidin-4-yl)-5-methylmorpholin-2-yl)methanol;
(4-(2-methoxy-6-(5-methyl-6-(1-(oxetan-3-yl)piperidin-4-yl)-1H-indazol-1-yl)pyrimidin-4-yl)-3-methylmorpholin-2-yl)methanol;
(4-(2-methyl-6-(5-methyl-6-(2-methyl-1-(oxetan-3-yl)piperidin-4-yl)-1H-indazol-1-yl)pyrimidin-4-yl)morpholin-2-yl)methanol;
(4-(2-methoxy-6-(5-methyl-6-(2-methyl-1-(oxetan-3-yl)piperidin-4-yl)-1H-indazol-1-yl)pyrimidin-4-yl)morpholin-2-yl)methanol;
   or a pharmaceutically acceptable salt thereof.

In one embodiment, the invention provides a compound of Formula (I-B) or a pharmaceutically acceptable salt thereof which is a compound of any one of Examples B-1 to B-28 or a pharmaceutically acceptable salt thereof. In one embodiment, the invention provides a compound of Formula (I-B) which is a compound of any one of Examples B-1 to B-28.

The present invention further provides compounds of Formula (I-C) and salts thereof:

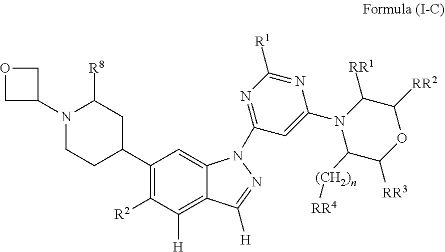

wherein
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃haloalkyl;
R³ is hydrogen or hydroxyl;
R⁸ is hydrogen or C₁₋₃alkyl;
RR¹, RR², and RR³ are independently hydrogen or C₁₋₃alkyl;
RR⁴ is hydrogen or hydroxyl; and
n is 1 or 2.

In another aspect of the invention, the invention provides a pharmaceutical composition comprising a compound of Formula (I-C) or a pharmaceutically acceptable salt thereof and a pharmaceutically acceptable carrier.

In one embodiment, R¹ is selected from the group consisting of C₁₋₃ alkyl and C₁₋₃ alkoxyl. In one embodiment, R¹ is selected from the group consisting of methyl or methoxy. In one embodiment, R¹ is methyl.

In one embodiment, R² is selected from the group consisting of H, halo and C₁₋₃alkyl. In one embodiment, R² is selected from the group consisting of C₁₋₃alkyl. In one embodiment, R² is selected from the group consisting of H, halo and methyl. In one embodiment, R² is selected from the group consisting of H, fluoro, chloro and methyl. In one embodiment, R² is selected from the group consisting of H, chloro and methyl. In one embodiment, R² is selected from the group consisting of chloro and methyl. In one embodiment, R² is methyl.

In one embodiment, RR¹ is hydrogen, RR² is hydrogen, RR³ is hydrogen, and R⁸ is hydrogen.

In one embodiment, RR¹ is hydrogen, RR² is hydrogen, RR³ is C₁₋₃alkyl, and R⁸ is hydrogen.

In one embodiment, RR¹ is hydrogen, RR² is hydrogen, RR³ is methyl, and R⁸ is hydrogen.

In one embodiment, n is 1.

In one embodiment, RR⁴ is hydrogen.

In one embodiment, RR⁴ is hydroxyl.

In one embodiment, the invention provides a compound of Formula (I-C) or a pharmaceutically acceptable salt thereof wherein n is 1. In this embodiment, R¹, R², RR¹, RR², RR³, RR⁴ and R⁸ may be further defined as in any of the preceding embodiments. For example, RR¹, RR², RR³ and R⁸ may be hydrogen.

In one embodiment, the invention provides a compound of Formula (I-C) or a pharmaceutically acceptable salt thereof which is a compound of any one of Examples C-1 to 6 or a pharmaceutically acceptable salt thereof. In one embodiment, the invention provides a compound of Formula (I-C) which is a compound of any one of Examples C-1 to C-6.

In addition to the free base form of the compounds described herein, the salt form of the compounds is also within the scope of the present invention. The salts or pharmaceutically-acceptable salts of the compounds described herein may be prepared in situ during the final isolation and purification of the compound, or by separately reacting the purified compound in its free base form with a suitable base or acid, respectively. For reviews on suitable pharmaceutical salts see Berge et al, J. Pharm, Sci., 66, 1-19, 1977; P L Gould, International Journal of Pharmaceutics, 33 (1986), 201-217; and Bighley et al, Encyclopedia of Pharmaceutical Technology, Marcel Dekker Inc, New York 1996, Volume 13, page 453-497.

Certain compounds of formula (I) contain a basic group and are therefore capable of forming pharmaceutically-acceptable acid addition salts by treatment with a suitable acid. Suitable acids include pharmaceutically-acceptable inorganic acids and pharmaceutically-acceptable organic acids. Exemplary pharmaceutically-acceptable acid addition salts include hydrochloride, hydrobromide, nitrate, methylnitrate, sulfate, bisulfate, sulfamate, phosphate, acetate, hydroxyacetate, phenylacetate, propionate, butyrate, isobutyrate, valerate, maleate, hydroxymaleate, acrylate, fumarate, malate, tartrate, citrate, salicylate, p-aminosalicyclate, glycollate, lactate, heptanoate, phthalate, oxalate, succinate, benzoate, o-acetoxybenzoate, chlorobenzoate, methylbenzoate, dinitrobenzoate, hydroxybenzoate, methoxybenzoate, mandelate, tannate, formate, stearate, ascorbate, palmitate, oleate, pyruvate, pamoate, malonate, laurate, glutarate, glutamate, estolate, methanesulfonate (mesylate), ethanesulfonate (esylate), 2-hydroxyethanesulfonate, benzenesulfonate (besylate), p-aminobenzenesulfonate, p-toluenesulfonate (tosylate), and napthalene-2-sulfonate. In some embodiments, the pharmaceutically acceptable salts include the L-tartrate, ethanedisulfonate (edisylate), sulfate, phosphate, p-toluenesulfonate (tosylate), hydrochloride salt, methanesulfonate, citrate, fumarate, benzenesulfonate, maleate, hydrobromate, L-lactate, malonate, and S-camphor-10-sulfonate. In certain embodiments, some of these salts form solvates. In certain embodiments, some of these salts are crystalline.

Certain compounds of Formula (I) or salts thereof may exist in stereoisomeric forms (e.g., they may contain one or more asymmetric carbon atoms). The individual stereoisomers (enantiomers and diastereomers) and mixtures of these are included within the scope of the present invention. The different isomeric forms may be separated or resolved one from the other by conventional methods, or any given isomer may be obtained by conventional synthetic methods or by stereospecific or asymmetric syntheses.

The invention also includes isotopically-labelled compounds and salts, which are identical to compounds of Formula (I) or salts thereof, but for the fact that one or more atoms are replaced by an atom having an atomic mass or mass number different from the atomic mass or mass number most commonly found in nature. Examples of isotopes that can be incorporated into compounds of Formula (I) or salts thereof isotopes of hydrogen, carbon, nitrogen, fluorine, such as ³H, ¹¹C, ¹⁴C and ¹⁸F. Such isotopically-labelled compound of Formula (I) or salts thereof are useful in drug and/or substrate tissue distribution assays. For example, ¹¹C and ¹⁸F isotopes are useful in PET (positron emission tomography). PET is useful in brain imaging. Isotopically-labelled compounds of Formula (I) and salts thereof can generally be prepared by carrying out the procedures disclosed below, by substituting a readily available isotopically-labelled reagent for a non-isotopically labelled reagent. In one embodiment, compounds of Formula (I) or salts thereof are not isotopically labelled.

Certain compounds of Formula (I) or salts thereof may exist in solid or liquid form. In the solid state, compounds of Formula (I) or salts may exist in crystalline or noncrystalline form, or as a mixture thereof. For compounds of Formula (I) or salts that are in crystalline form, the skilled artisan will appreciate that pharmaceutically-acceptable solvates may be formed wherein solvent molecules are incorporated into the crystalline lattice during crystallization. Solvates may involve nonaqueous solvents such as ethanol, isopropanol, DMSO, acetic acid, ethanolamine, and ethyl acetate, or they may involve water as the solvent that is incorporated into the crystalline lattice. Solvates wherein water is the solvent that is incorporated into the crystalline lattice are typically referred to as “hydrates.” Hydrates include stoichiometric hydrates as well as compositions containing variable amounts of water.

The skilled artisan will further appreciate that certain compounds of Formula (I), pharmaceutically acceptable salts or solvates thereof that exist in crystalline form, including the various solvates thereof, may exhibit polymorphism (i.e. the capacity to occur in different crystalline structures). These different crystalline forms are typically known as “polymorphs.” Polymorphs have the same chemical composition but differ in packing, geometrical arrangement, and other descriptive properties of the crystalline solid state. Polymorphs, therefore, may have different physical properties such as shape, density, hardness, deformability, stability, and dissolution properties. Polymorphs typically exhibit different melting points, IR spectra, and X-ray powder diffraction patterns, which may be used for identification. The skilled artisan will appreciate that different polymorphs may be produced, for example, by changing or adjusting the reaction conditions or reagents, used in making the compound. For example, changes in temperature, pressure, or solvent may result in polymorphs. In addition, one polymorph may spontaneously convert to another polymorph under certain conditions.

The skilled artisan also appreciates that this invention may contain various deuterated forms of compounds of Formula (I), or pharmaceutically acceptable salts thereof. Each available hydrogen atom attached to a carbon atom may be independently replaced with a deuterium atom. A person of ordinary skill in the art will know how to synthesize deuterated forms of compounds of Formula (I), or pharmaceutically acceptable salts thereof. Commercially available deuterated starting materials may be employed in the preparation of deuterated forms of compounds of Formula (I) or pharmaceutically acceptable salts thereof, or they may be synthesized using conventional techniques employing deuterated reagents (e.g. lithium aluminum deuteride).

### C. Methods of Use

Compounds of Formula (I) or pharmaceutically acceptable salts thereof are inhibitors of LRRK2 kinase activity and are thus believed to be of potential use in the treatment of or prevention of the following neurological diseases: Parkinson's disease, Alzheimer's disease, dementia (including Lewy body dementia and vascular dementia, HIV-induced dementia), amyotrophic lateral sclerosis (ALS), age related memory dysfunction, mild cognitive impairment, argyrophilic grain disease, Pick's disease, corticobasal degeneration, progressive supranuclear palsy, inherited frontotemporal dementia and parkinsonism linked to chromosome 17 (FTDP-17), withdrawal symptoms/relapse associated with drug addiction, L-Dopa induced dyskinesia, ischemic stroke, traumatic brain injury, spinal cord injury and multiple sclerosis. Other diseases potentially treatable by inhibition of LRRK2 include, but are not limited to, lysosomal disorders (for example, Niemann-Pick Type C disease, Gaucher disease), Crohn's disease, cancers (including thyroid, renal (including papillary renal), breast, lung and prostate cancers, leukemias (including acute myelogenous leukemia (AML)) and lymphomas), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, obesity, epilepsy, pulmonary diseases such as chronic obstructive pulmonary disease, idiopathic pulmonary fibrosis, Sjogren's syndrome, Devic's disease, inflammatory myopathies, ankylosing spondylitis, bacterial infections (including leprosy), viral infections (including tuberculosis, HIV, West Nile virus and chikungunya virus) and parasitic infections.

One aspect of the invention provides a compound of Formula (I) or a pharmaceutically acceptable salt thereof for use in therapy. In one embodiment, the invention provides a compound of Formula (I) or a pharmaceutically acceptable salt thereof for use in the treatment of or prevention of the above disorders (i.e. the neurological diseases and other diseases listed above). In one embodiment, the invention provides a compound of Formula (I) or a pharmaceutically acceptable salt thereof for use in the treatment of or prevention of Parkinson's disease. In one embodiment, the invention provides a compound of Formula (I) or a pharmaceutically acceptable salt thereof for use in the treatment of Parkinson's disease. In another embodiment, the invention provides a compound of Formula (I) or a pharmaceutically acceptable salt thereof for use in the treatment of or prevention of Alzheimer's disease. In one embodiment, the invention provides a compound of Formula (I) or a pharmaceutically acceptable salt thereof for use in the treatment of Alzheimer's disease. In another embodiment, the invention provides a compound of Formula (I) or a pharmaceutically acceptable salt thereof for use in the treatment of amyotrophic lateral sclerosis (ALS).

In one embodiment, the invention provides a compound of Formula (I), Formula (I-A), Formula (I-B), or Formula (I-C), or a pharmaceutically acceptable salt thereof for use in the treatment or prevention of Parkinson's disease, Alzheimer's disease or amyotrophic lateral sclerosis (ALS).

In another embodiment, the invention provides a compound of Formula (I), Formula (I-A), Formula (I-B), or Formula (I-C), or a pharmaceutically acceptable salt thereof for use in the treatment of Parkinson's disease.

A further aspect of the invention provides the use of a compound of Formula (I) or a pharmaceutically acceptable salt thereof in the manufacture of a medicament for the treatment or prevention of the above disorders (i.e. the neurological diseases and other diseases listed above). A further aspect of the invention provides the use of a compound of Formula (I) or a pharmaceutically acceptable salt thereof in the manufacture of a medicament for the treatment of or prevention of Parkinson's disease. A further aspect of the invention provides the use of a compound of Formula (I) or a pharmaceutically acceptable salt thereof in the manufacture of a medicament for the treatment of Parkinson's disease. In another embodiment, the invention provides the use of a compound of Formula (I) or a pharmaceutically acceptable salt thereof in the manufacture of a medicament for the treatment or prevention of Alzheimer's disease. In one embodiment, the invention provides the use of a compound of Formula (I) or a pharmaceutically acceptable salt thereof in the manufacture of a medicament for the treatment of Alzheimer's disease. In another embodiment, the invention provides use of a compound of Formula (I) or a pharmaceutically acceptable salt thereof in the manufacture of a medicament for the treatment of amyotrophic lateral sclerosis (ALS).

In one embodiment, the invention provides the use of a compound of Formula (I), Formula (I-A), Formula (I-B), or Formula (I-C), or a pharmaceutically acceptable salt thereof in the manufacture of a medicament for the treatment or prevention of Parkinson's disease, Alzheimer's disease or amyotrophic lateral sclerosis (ALS).

In another embodiment, the invention provides the use of a compound of Formula (I), Formula (I-A), Formula (I-B), or Formula (I-C), a pharmaceutically acceptable salt thereof in the manufacture of a medicament for the treatment or prevention of Parkinson's disease.

In yet another embodiment, the invention provides the use of a compound of Formula (I), Formula (I-A), Formula (I-B), or Formula (I-C), or a pharmaceutically acceptable salt thereof in the manufacture of a medicament for the treatment of Parkinson's disease.

A further aspect of the invention provides a method of treatment or prevention of a disorder listed above (i.e. selected from the neurological diseases and other diseases listed above), which comprises administering to a subject in need thereof a therapeutically effective amount of a compound of Formula (I) or a pharmaceutically acceptable salt thereof. A further aspect of the invention provides a method of treatment or prevention of Parkinson's disease, which comprises administering to a subject in need thereof a therapeutically effective amount of a compound of Formula (I) or a pharmaceutically acceptable salt thereof. A further aspect of the invention provides a method of treatment of Parkinson's disease, which comprises administering to a subject in need thereof a therapeutically effective amount of a compound of Formula (I) or a pharmaceutically acceptable salt thereof. A further aspect of the invention provides a method of treatment or prevention of Alzheimer's disease, which comprises administering to a subject in need thereof a therapeutically effective amount of a compound of Formula (I) or a pharmaceutically acceptable salt thereof. A further aspect of the invention provides a method of treatment of Alzheimer's disease, which comprises administering to a subject in need thereof a therapeutically effective amount of a compound of Formula (I) or a pharmaceutically acceptable salt thereof. A further aspect of the invention provides a method of treatment of tuberculosis, which comprises administering to a subject in need thereof a therapeutically effective amount of a compound of Formula (I) or a pharmaceutically acceptable salt thereof. In an embodiment, the subject is human.

In one embodiment, the invention provides a method of treatment of Parkinson's disease, Alzheimer's disease or amyotrophic lateral sclerosis (ALS), which comprises administering to a subject in need thereof a therapeutically effective amount of a compound of Formula (I) or a pharmaceutically acceptable salt thereof.

In one embodiment, the invention provides a method of treatment of Parkinson's disease, Alzheimer's disease or amyotrophic lateral sclerosis (ALS), which comprises administering to a human in need thereof a therapeutically effective amount of a compound of Formula (I) or a pharmaceutically acceptable salt thereof.

In one embodiment, the invention provides a method of treatment of Parkinson's disease, which comprises administering to a subject in need thereof a therapeutically effective amount of a compound of Formula (I) or a pharmaceutically acceptable salt thereof.

In one embodiment, the invention provides a method of treatment of Parkinson's disease, which comprises administering to a human in need thereof a therapeutically effective amount of a compound of Formula (I) or a pharmaceutically acceptable salt thereof.

In the context of the present invention, treatment of Parkinson's disease refers to the treatment of sporadic Parkinson's disease, and/or familial Parkinson's disease. In one embodiment, treatment of Parkinson's disease refers to treatment of familial Parkinson's disease. Familial Parkinson's disease patients are those expressing one or more of the following LRRK2 kinase mutations: G2019S mutation, N1437H mutation, R1441G mutation, R1441C mutation, R1441H mutation, Y1699C mutation, S1761R mutation, or 12020T mutation. In another embodiment, familial Parkinson's disease patients express other coding mutations (such as G2385R) or non-coding single nucleotide polymorphisms at the LRRK2 locus that are associated with Parkinson's disease. In a more particular embodiment, familial Parkinson's disease includes patients expressing the G2019S mutation or the R1441G mutation in LRRK2 kinase. In one embodiment, treatment of Parkinson's disease refers to the treatment of familial Parkinson's disease includes patients expressing LRRK2 kinase bearing G2019S mutation. In another embodiment, familial Parkinson's disease patients express aberrantly high levels of normal LRRK2 kinase.

In one embodiment, the invention provides a method of treatment of Parkinson's disease, which comprises administering to a human expressing the G2019S mutation in LRRK2 kinase in need thereof a therapeutically effective amount of a compound of Formula (I) or a pharmaceutically acceptable salt thereof.

In one embodiment, the invention provides a method of treatment of Parkinson's disease, which comprises testing in a human for the G2019S mutation in LRRK2 kinase and administering to the human expressing the G2019S mutation in LRRK2 kinase in need thereof a therapeutically effective amount of a compound of Formula (I) or a pharmaceutically acceptable salt thereof.

Treatment of Parkinson's disease may be symptomatic or may be disease modifying. In one embodiment, treatment of Parkinson's disease refers to symptomatic treatment. In one embodiment, treatment of Parkinson's disease refers to disease modifying treatment.

Compounds of the present invention may also be useful in treating patients identified as susceptible to progression to severe Parkinsonism by means of one or more subtle features associated with disease progression such as family history, olfaction deficits, constipation, cognitive defects, gait or biological indicators of disease progression gained from molecular, biochemical, immunological or imaging technologies. In this context, treatment may be symptomatic or disease modifying.

In the context of the present invention, treatment of Alzheimer's disease refers to the treatment of sporadic Alzheimer's disease and/or familial Alzheimer's disease. Treatment of Alzheimer's disease may be symptomatic or may be disease modifying. In one embodiment, treatment of Alzheimer's disease refers to symptomatic treatment.

In the context of the present invention, treatment of dementia (including Lewy body dementia and vascular dementia, HIV-induced dementia), amyotrophic lateral sclerosis (ALS), age related memory dysfunction, mild cognitive impairment, argyrophilic grain disease, Pick's disease, corticobasal degeneration, progressive supranuclear palsy, inherited frontotemporal dementia and parkinsonism linked to chromosome 17 (FTDP-17), multiple sclerosis, lysosomal disorders (for example, Niemann-Pick Type C disease, Gaucher disease), Crohn's disease, cancers (including thyroid, renal (including papillary renal), breast, lung and prostate cancers, leukemias (including acute myelogenous leukemia (AML)) and lymphomas), rheumatoid arthritis, systemic lupus erythematosus, autoimmune hemolytic anemia, pure red cell aplasia, idiopathic thrombocytopenic purpura (ITP), Evans syndrome, vasculitis, bullous skin disorders, type 1 diabetes mellitus, obesity, epilepsy, pulmonary diseases such as chronic obstructive pulmonary disease, idiopathic pulmonary fibrosis, Sjogren's syndrome, Devic's disease, inflammatory myopathies, ankylosing spondylitis, may be symptomatic or disease modifying. In certain embodiments, treatment of these disorders refers to symptomatic treatment.

The invention also provides the use of inhibitors of LRRK2 in the production of neuronal progenitor cells in vitro for consequent therapeutic application in cell based-treatment of CNS disorders.

When a compound of Formula (I) or a pharmaceutically acceptable salt thereof is intended for use in the treatment of Parkinson's disease, it may be used in combination with medicaments alleged to be useful as symptomatic treatments of Parkinson's disease. Suitable examples of such other therapeutic agents include L-dopa, and dopamine agonists (e.g. pramipexole, ropinirole).

When a compound of Formula (I) or a pharmaceutically acceptable salt thereof is intended for use in the treatment of Alzheimer's disease, it may be used in combination with medicaments claimed to be useful as either disease modifying or symptomatic treatments of Alzheimer's disease. Suitable examples of such other therapeutic agents may be symptomatic agents, for example those known to modify cholinergic transmission such as M1 muscarinic receptor agonists or allosteric modulators, M2 muscarinic antagonists, acetylcholinesterase inhibitors (such as tetrahydroaminoacridine, donepezil hydrochloride rivastigmine, and galantamine), nicotinic receptor agonists or allosteric modulators (such as α7 agonists or allosteric modulators or α4β2 agonists or allosteric modulators), PPAR agonists (such as PPARγ agonists), 5-HT₄ receptor partial agonists, 5-HT₆ receptor antagonists e.g. SB-742457 or 5HT1A receptor antagonists and NMDA receptor antagonists or modulators, or disease modifying agents such as β or γ-secretase inhibitors e.g semagacestat, mitochondrial stabilizers, microtubule stabilizers or modulators of Tau pathology such as Tau aggregation inhibitors (e.g. methylene blue and REMBER™), NSAIDS, e.g. tarenflurbil, tramiprosil; or antibodies for example bapineuzumab or solanezumab; proteoglycans for example tramiprosate.

When a compound of Formula (I) or a pharmaceutically acceptable salt thereof is intended for use in the treatment of bacterial infections, parasitic infections or viral infections, it may be used in combination with medicaments alleged to be useful as symptomatic treatments that directly target the infectious agent.

When a compound of Formula (I) or a pharmaceutically acceptable salt thereof is used in combination with other therapeutic agents, the compound may be administered either sequentially or simultaneously by any convenient route.

The invention also provides, in a further aspect, a combination comprising a compound of Formula (I) or a pharmaceutically acceptable salt thereof together with one or more further therapeutic agent or agents.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of Formula (I) or a pharmaceutically acceptable salt thereof is used in combination with a second therapeutic agent active against the same disease state the dose of each compound may differ from that when the compound is used alone. Appropriate doses will be readily appreciated by those skilled in the art.

### D. Composition

Compounds of Formula (I) or pharmaceutically acceptable salts thereof may be formulated into pharmaceutical compositions prior to administration to a subject. According to one aspect, the invention provides a pharmaceutical composition comprising a compound of Formula (I) or a pharmaceutically acceptable salt thereof and a pharmaceutically acceptable excipient. According to another aspect, the invention provides a process for the preparation of a pharmaceutical composition comprising admixing a compound of Formula (I) or a pharmaceutically acceptable salt thereof, with a pharmaceutically acceptable excipient.

Pharmaceutical compositions may be presented in unit dose forms containing a predetermined amount of active ingredient per unit dose. Such a unit may contain, for example, 0.1 mg, 0.5 mg, or 1 mg to 50 mg, 100 mg, 200 mg, 250 mg, 500 mg, 750 mg or 1 g of a compound of the present invention, depending on the disease being treated, the route of administration and the age, weight and condition of the subject, or pharmaceutical compositions may be presented in unit dose forms containing a predetermined amount of active ingredient per unit dose. In other embodiments, the unit dosage compositions are those containing a daily dose or sub-dose as described herein, or an appropriate fraction thereof, of an active ingredient. Furthermore, such pharmaceutical compositions may be prepared by any of the methods well-known to one skilled in the art.

A therapeutically effective amount of a compound of Formula (I) will depend upon a number of factors including, for example, the age and weight of the intended recipient, the precise condition requiring treatment and its severity, the nature of the formulation, and the route of administration, and will ultimately be at the discretion of the attendant prescribing the medication. However, a therapeutically effective amount of a compound of formula (I) for the treatment of diseases described in the present invention will generally be in the range of 0.1 to 100 mg/kg body weight of recipient per day and more usually in the range of 1 to 10 mg/kg body weight per day. Thus, for a 70 kg adult mammal, the actual amount per day would usually be from 70 to 700 mg and this amount may be given in a single dose per day or in a number of sub-doses per day as such as two, three, four, five or six doses per day. Or the dosing can be done intermittently, such as once every other day, once a week or once a month. A therapeutically effective amount of a pharmaceutically acceptable salt or solvate, etc., may be determined as a proportion of the therapeutically effective amount of the compound of Formula (I) per se. It is envisaged that similar dosages would be appropriate for treatment of the other diseases referred to above.

The pharmaceutical compositions of the invention may contain one or more compounds of Formula (I) or a pharmaceutically acceptable salt thereof. In some embodiments, the pharmaceutical compositions may contain more than one compound of the invention. For example, in some embodiments, the pharmaceutical compositions may contain two or more compounds of Formula (I) or a pharmaceutically acceptable salt thereof. In addition, the pharmaceutical compositions may optionally further comprise one or more additional active pharmaceutical ingredients (APIs).

As used herein, “pharmaceutically acceptable excipient” means a pharmaceutically acceptable material, composition or vehicle involved in giving form or consistency to the pharmaceutical composition. Each excipient may be compatible with the other ingredients of the pharmaceutical composition when commingled such that interactions which would substantially reduce the efficacy of the compound of the invention when administered to a subject and interactions which would result in pharmaceutical compositions that are not pharmaceutically acceptable are avoided.

The compounds of the invention and the pharmaceutically-acceptable excipient or excipients may be formulated into a dosage form adapted for administration to the subject by the desired route of administration. For example, dosage forms include those adapted for (1) oral administration (including buccal or sublingual) such as tablets, capsules, caplets, pills, troches, powders, syrups, elixers, suspensions, solutions, emulsions, sachets, and cachets; (2) parenteral administration (including subcutaneous, intramuscular, intravenous or intradermal) such as sterile solutions, suspensions, and powders for reconstitution; (3) transdermal administration such as transdermal patches; (4) rectal administration such as suppositories; (5) nasal inhalation such as dry powders, aerosols, suspensions, and solutions; and (6) topical administration (including buccal, sublingual or transdermal) such as creams, ointments, lotions, solutions, pastes, sprays, foams, and gels. Such compositions may be prepared by any methods known in the art of pharmacy, for example by bringing into association a compound of Formula (I) with the carrier(s) or excipient(s).

Pharmaceutical compositions adapted for oral administration may be presented as discrete units such as capsules or tablets; powders or granules; solutions or suspensions in aqueous or non-aqueous liquids; edible foams or whips; or oil-in-water liquid emulsions or water-in-oil liquid emulsions.

Suitable pharmaceutically-acceptable excipients may vary depending upon the particular dosage form chosen. In addition, suitable pharmaceutically-acceptable excipients may be chosen for a particular function that they may serve in the composition. For example, certain pharmaceutically-acceptable excipients may be chosen for their ability to facilitate the production of uniform dosage forms. Certain pharmaceutically-acceptable excipients may be chosen for their ability to facilitate the production of stable dosage forms. Certain pharmaceutically acceptable excipients may be chosen for their ability to facilitate carrying or transporting the compound or compounds of the invention once administered to the subject from an organ, or a portion of the body, to another organ, or a portion of the body. Certain pharmaceutically-acceptable excipients may be chosen for their ability to enhance patient compliance.

Suitable pharmaceutically acceptable excipients include the following types of excipients: diluents, fillers, binders, disintegrants, lubricants, glidants, granulating agents, coating agents, wetting agents, solvents, co-solvents, suspending agents, emulsifiers, sweeteners, flavoring agents, flavor masking agents, coloring agents, anticaking agents, hemectants, chelating agents, plasticizers, viscosity increasing agents, antioxidants, preservatives, stabilizers, surfactants, and buffering agents. The skilled artisan will appreciate that certain pharmaceutically-acceptable excipients may serve more than one function and may serve alternative functions depending on how much the excipient is present in the formulation and what other ingredients are present in the formulation.

Skilled artisans possess the knowledge and skill in the art to enable them to select suitable pharmaceutically-acceptable excipients in appropriate amounts for use in the invention. In addition, there are a number of resources that are available to the skilled artisan which describe pharmaceutically-acceptable excipients and may be useful in selecting suitable pharmaceutically-acceptable excipients. Examples include Remington's Pharmaceutical Sciences (Mack Publishing Company), The Handbook of Pharmaceutical Additives (Gower Publishing Limited), and The Handbook of Pharmaceutical Excipients (the American Pharmaceutical Association and the Pharmaceutical Press).

The pharmaceutical compositions of the invention are prepared using techniques and methods known to those skilled in the art. Some of the methods commonly used in the art are described in Remington's Pharmaceutical Sciences (Mack Publishing Company).

In one aspect, the invention is directed to a solid oral dosage form such as a tablet or capsule comprising a therapeutically effective amount of a compound of the invention and a diluent or filler. Suitable diluents and fillers include lactose, sucrose, dextrose, mannitol, sorbitol, starch (e.g. corn starch, potato starch, and pre-gelatinized starch), cellulose and its derivatives (e.g. microcrystalline cellulose), calcium sulfate, and dibasic calcium phosphate. The oral solid dosage form may further comprise a binder. Suitable binders include starch (e.g. corn starch, potato starch, and pre-gelatinized starch), gelatin, acacia, sodium alginate, alginic acid, tragacanth, guar gum, povidone, and cellulose and its derivatives (e.g. microcrystalline cellulose). The oral solid dosage form may further comprise a disintegrant. Suitable disintegrants include crospovidone, sodium starch glycolate, croscarmellose, alginic acid, and sodium carboxymethyl cellulose. The oral solid dosage form may further comprise a lubricant. Suitable lubricants include stearic acid, magnesium stearate, calcium stearate, and talc.

In certain embodiments, the present invention is directed to a pharmaceutical composition comprising 0.01 to 1000 mg of one or more of a compound of Formula (I) or a pharmaceutically acceptable salt thereof and 0.01 to 5 g of one or more pharmaceutically acceptable excipients.

In another embodiment, the present invention is directed to a pharmaceutical composition for the treatment of a neurodegeneration disease comprising a compound of formula (I) or a pharmaceutically acceptable salt thereof and a pharmaceutically acceptable excipient. In another embodiment, the present invention is directed to a pharmaceutical composition for the treatment of Parkinson's disease comprising a compound of formula (I) or a pharmaceutically acceptable salt thereof and a pharmaceutically acceptable excipient.

### E. Process of Preparing Compounds

The process to be utilized in the preparation of compounds of formula (I) or salts thereof described herein depends upon the desired compounds. Such factors as the selection of the specific substituent and various possible locations of the specific substituent all play a role in the path to be followed in the preparation of the specific compounds of this invention. Those factors are readily recognized by one of ordinary skill in the art.

In general, the compounds of the present invention may be prepared by standard techniques known in the art and by known processes analogous thereto. General methods for preparing compounds of formula (I) are set forth below. All starting material and reagents described in the below general experimental schemes are commercially available or can be prepared by methods known to one skilled in the art.

The skilled artisan will appreciate that if a substituent described herein is not compatible with the synthetic methods described herein, the substituent may be protected with a suitable protecting group that is stable to the reaction conditions. The protecting group may be removed at a suitable point in the reaction sequence to provide a desired intermediate or target compound. Suitable protecting groups and the methods for protecting and de-protecting different substituents using such suitable protecting groups are well known to those skilled in the art; examples of which may be found in T. Greene and P. Wuts, Protecting Groups in Chemical Synthesis (3rd ed.), John Wiley & Sons, NY (1999). In some instances, a substituent may be specifically selected to be reactive under the reaction conditions used. Under these circumstances, the reaction conditions convert the selected substituent into another substituent that is either useful as an intermediate compound or is a desired substituent in a target compound.

General Scheme A-1 provides exemplary processes of synthesis for preparing compounds of the present invention.

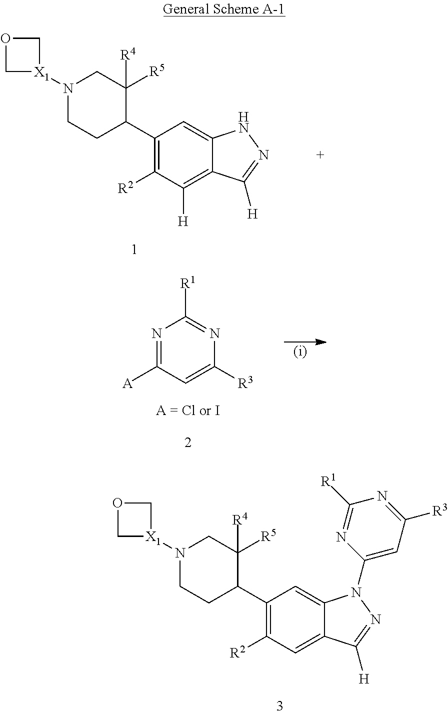

General Scheme A-1 provides an exemplary synthesis for preparing compound 3 which represents compounds of Formula (I-A). In Scheme A-1, R¹, R², R³, R⁴, R⁵ and X₁ are as defined in Formula (I-A).

Step (i) may be a substitution reaction by reacting compound 1 with compound 2 using appropriate base such as Cs₂CO₃ in an appropriate solvent such as N, N-dimethylformamide (DMF) under suitable temperature such as about 100° C. to provide compound 3.

Step (i) may alternatively be a coupling reaction using appropriate reagents such as CuI and N,N′-dimethyl-cyclohexane-1,2-diamine in the presence of suitable base such as K₃PO₄ in a suitable solvent such as toluene at suitable temperature such as reflux condition to provide compound 3.

Step (i) may alternatively be a coupling reaction using appropriate reagents such as Pd₂dba₃ and di-tert-butyl(2′,4′,6′-triisopropyl-[1,1′-biphenyl]-2-yl)phosphine in the presence of suitable base such as sodium tert-butoxide in a suitable solvent such as toluene at suitable temperature such as 100° C. to provide compound 3.

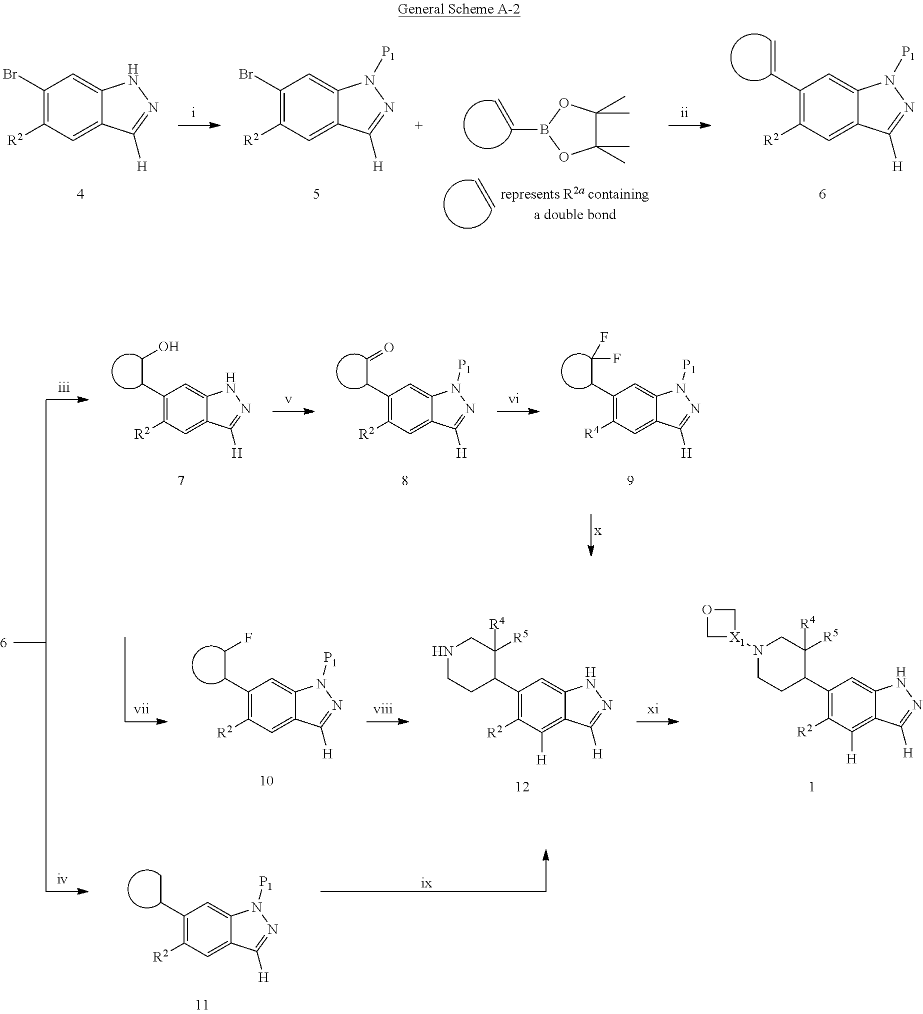

General Scheme A-2 provides an exemplary synthesis for preparing intermediate 1. The protecting group, P₁, can be any suitable protecting groups for example, tetrahydro-2H-pyran-2-yl (THP), (trimethylsilyl)ethoxy)methyl (SEM) or Acetyl (Ac).

Intermediate 5 can be obtained in step (i) by reacting starting material 4 with suitable reagents such as DHP in the presence of suitable acids such as TsOH in appropriate solvents such as DCM under suitable temperatures such as 20° C. to 40° C.

Step (ii) is a cross-coupling reaction between intermediate 5 and boronic acid or esters using appropriate palladium catalysts such as Pd(dppf)Cl₂ in the presence of suitable bases such as Na₂CO₃ in appropriate solvents such as 1,4-dioxane at suitable temperatures such as 60° C. to 100° C.

Step (iii) involves reaction with suitable oxidation reagents such as H₂O₂ in a suitable solvent such as THF under suitable temperatures such as −60° C. to −10° C. to provide intermediate 7.

Step (iv) is a reaction with a suitable reducing reagent such as hydrogen in the presence of suitable catalysts such Pd/C in polar solvents such as MeOH at appropriate temperatures such as 25° C. to 80° C.

Step (v) may be an oxidation reaction with oxidants such as DMP in suitable solvents such as DCM under suitable temperatures such as 0° C. to 25° C. to give intermediate 8.

Steps (vi) and (vii) involve reaction with a fluridizer such as DAST in suitable solvents such as DCM under suitable temperatures such as −78° C. to 0° C.

Steps (viii) (ix) and (x) are de-protection reactions. Typically, the intermediate is reacted with suitable acids such HCl in suitable solvents such as 1,4-dioxane under suitable temperatures such as 25° C. to 40° C. to give intermediate 1.

Step (xi) involves reaction with substituted oxetan-3-one under suitable reductant such as NaBH₃CN in a suitable solvent such as MeOH and CH₂Cl₂ at suitable temperature such as room temperature.

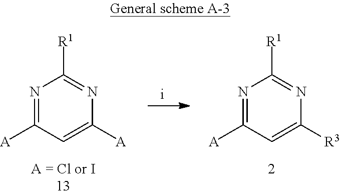

General Scheme A-3 provides an exemplary synthesis for preparing intermediates 2.

When R³ is an N-linked 4-6 membered heterocyclyl ring or NHR⁷; step (i) can be a reaction with different amines using appropriate bases such as TEA in appropriate solvents such as EtOH under suitable temperatures such as 25° C. to 100° C. to provide intermediate 2.

When R₃ is OR⁷, step (i) is a coupling reaction. The alcohol (R⁷OH) is deprotonated by a suitable base such as sodium hydride in suitable solvent such as THF at suitable temperature such as 0° C. to give the transitional intermediate. Then intermediate 13 is reacted with the transitional intermediate in suitable solvent such as THF at suitable temperature such as room temperature.

General Scheme B-1 provides exemplary processes of synthesis for preparing compounds of Formula (I-B).

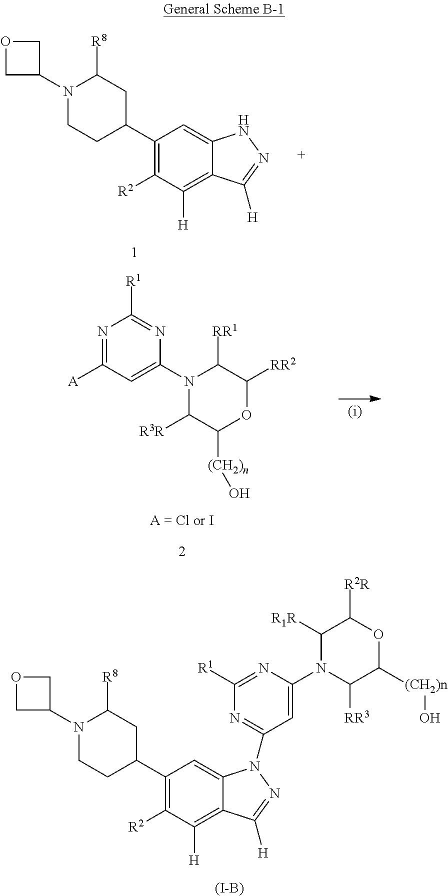

General Scheme B-1 provides an exemplary synthesis for preparing compounds of Formula (I-B). In Scheme 1, R¹, R², RR¹, RR², RR³ and R⁸ and n are as defined in Formula (I-B).

Step (i) may be a substitution reaction by reacting compound 1 with compound 2 using appropriate base such as Cs₂CO₃ in an appropriate solvent such as N, N-dimethylformamide (DMF) under suitable temperature such as about 100° C. to provide a compound of Formula (I-B).

Step (i) may alternatively be a coupling reaction using appropriate reagents such as CuI and N,N′-dimethyl-cyclohexane-1,2-diamine in the presence of suitable base such as K₃PO₄ in a suitable solvent such as toluene at suitable temperature such as reflux condition to provide a compound of Formula (I-B).

Step (i) may alternatively be a coupling reaction using appropriate reagents such as Pd₂dba₃ and di-tert-butyl(2′,4′,6′-triisopropyl-[1,1′-biphenyl]-2-yl)phosphine in the presence of suitable base such as sodium tert-butoxide in a suitable solvent such as toluene at suitable temperature such as 100° C. to provide a compound of Formula (I-B).

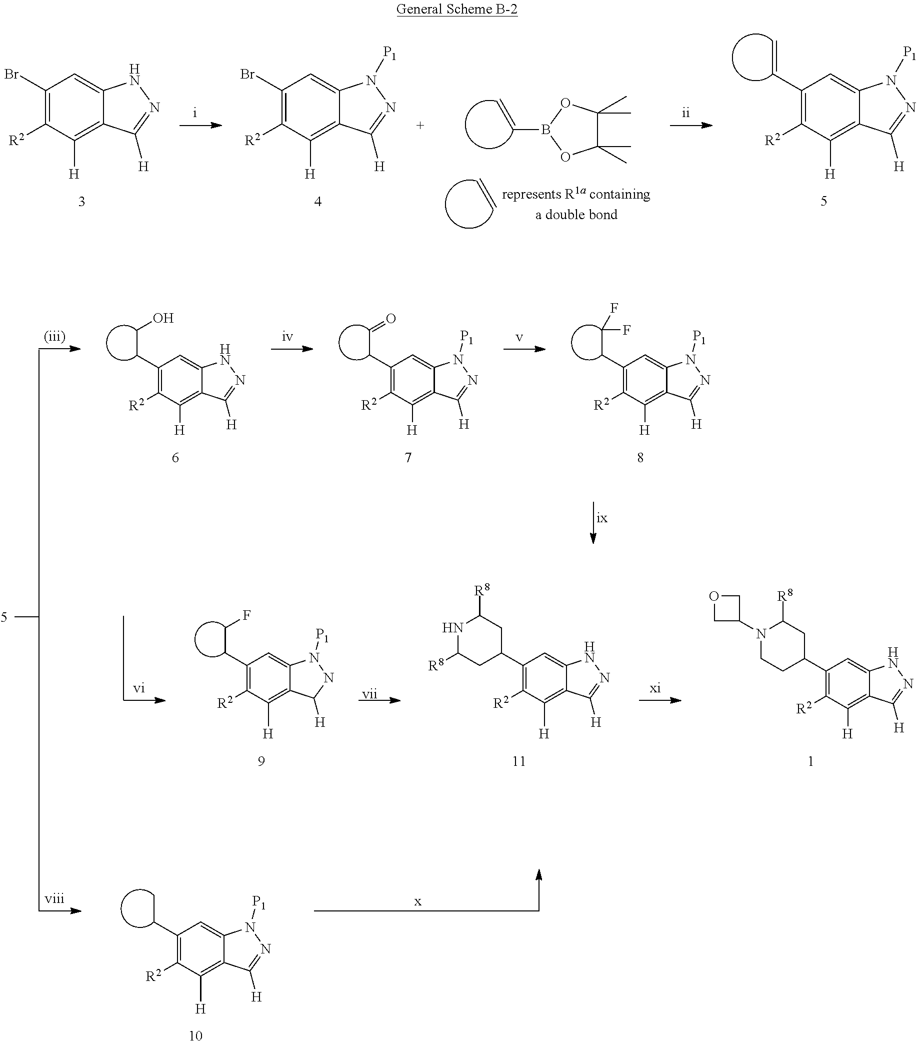

General Scheme B-2 provides an exemplary synthesis for preparing intermediate 1. The protecting group P₁ can be hydrogen or any suitable protecting group for example, tetrahydro-2H-pyran-2-yl (THP), (trimethylsilyl)ethoxy)methyl (SEM) or Acetyl (Ac).

Intermediates 4 can be obtained in step (i) by reacting starting material 3 with suitable reagents such as DHP in the presence of suitable acids such as TsOH in appropriate solvents such as DCM under suitable temperatures such as 20° C. to 40° C.

Step (ii) may be a cross-coupling reaction between intermediate 4 with suitable reagents such as boronic acid or esters using appropriate palladium catalysts such as Pd(dppf)Cl₂ in the presence of suitable bases such as Na₂CO₃ in appropriate solvents such as 1,4-dioxane under suitable temperatures such as 60° C. to 100° C.

Step (iii) is a reaction with suitable oxidation reagents such as H₂O₂ in a suitable solvent such as THF at a suitable temperature such as −60° C. to −10° C.

Step (iv) may be an oxidation reaction involving reacting intermediate 6 with oxidants such as DMP in suitable solvents such as DCM under suitable temperatures such as 0° C. to 25° C. to give intermediate7.

Steps (v) and (vi) involve a reaction with a fluridizer such as DAST in a suitable solvent such as DCM at a suitable temperature such as −78° C. to 0° C.

Step (viii) is a reaction with a suitable reducing reagent such as hydrogen in the presence of a suitable catalyst such Pd/C in polar solvents such as MeOH at an appropriate temperature such as 25° C. to 80° C.

Steps (vii), (ix) and (x) are de-protection reactions typically involving a reaction with a suitable acid such HCl in a suitable solvent such as 1,4-dioxane at a suitable temperature such as 25° C. to 40° C.

Step (xi) is a coupling reaction with oxetan-3-one using appropriate reagents in the presence of suitable base such as NaBH₃CN in the presence of a catalyst for example AcOH, in a suitable solvent such as DCM or DCE at suitable temperature such as room temperature.

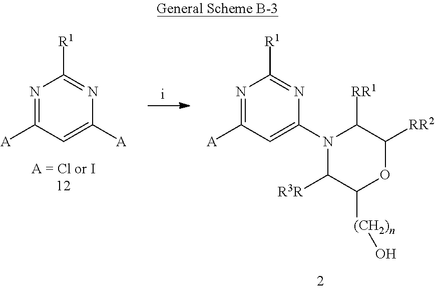

General Scheme B-3 provides an exemplary synthesis for preparing intermediate 2, wherein R¹, RR¹, RR², RR³ and n are as shown in compounds of Formula (I-B) Step (i) can be a reaction between intermediate 12 with different amines such as morpholine using appropriate bases such as TEA in appropriate solvents such as EtOH under suitable temperatures such as 25° C. to 100° C. to provide intermediate 2.

Intermediate 2 can be also obtained by a coupling reaction between intermediate 12 with suitable reagents such as boronic acid in the presence of catalysts such as Pd(PPh₃)₂Cl₂ in suitable solvents such as 1,4-dioxane under 25° C. to 130° C. in step (xx).

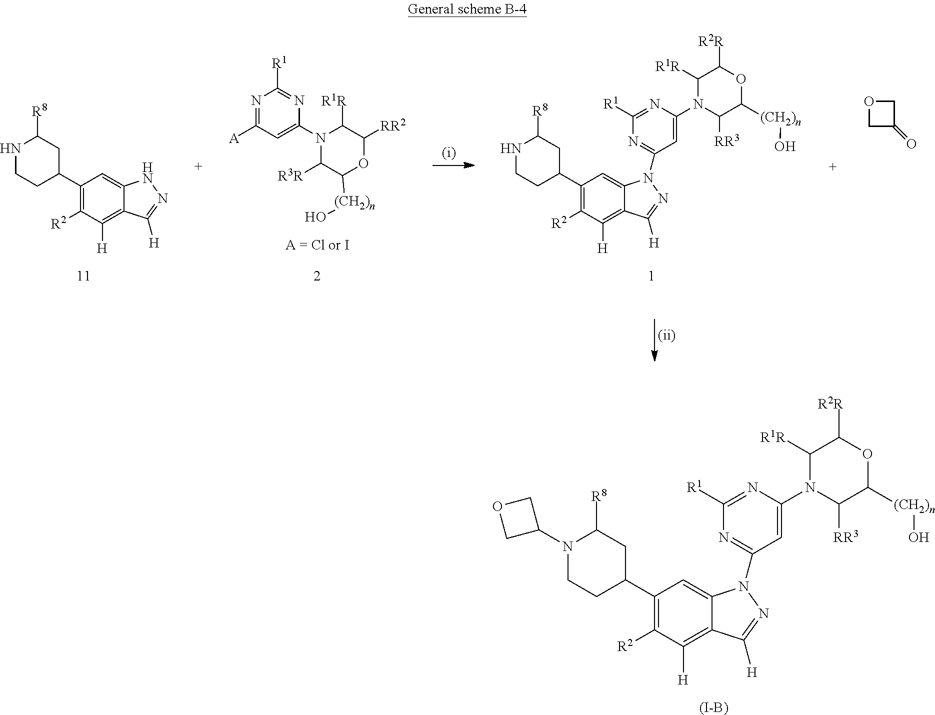

General Scheme B-4 provides an alternative exemplary synthesis for preparing compounds of Formula (I-B). In Scheme 4, R¹, R², RR¹, RR², RR³ and R⁸ and n are as defined in Formula (I-B).

Step (i) may be a substitution reaction using an appropriate base such as Cs₂CO₃ in an appropriate solvent such as N, N-dimethylformamide (DMF) under suitable temperature such as about 100° C.

Step (i) may alternatively be a coupling reaction using appropriate reagents such as CuI and N,N′-dimethyl-cyclohexane-1,2-diamine in the presence of suitable base such as K₃PO₄ in a suitable solvent such as toluene at suitable temperature such as reflux condition.

Step (ii) may be a coupling reaction using appropriate reagents in the presence of suitable base such as NaBH₃CN in the presence of a catalyst for example AcOH, in a suitable solvent such as DCM or DCE at suitable temperature such as room temperature to provide a compound of Formula (I-B).

General Scheme C-1 provides exemplary processes of synthesis for preparing compounds of Formula (I-C).

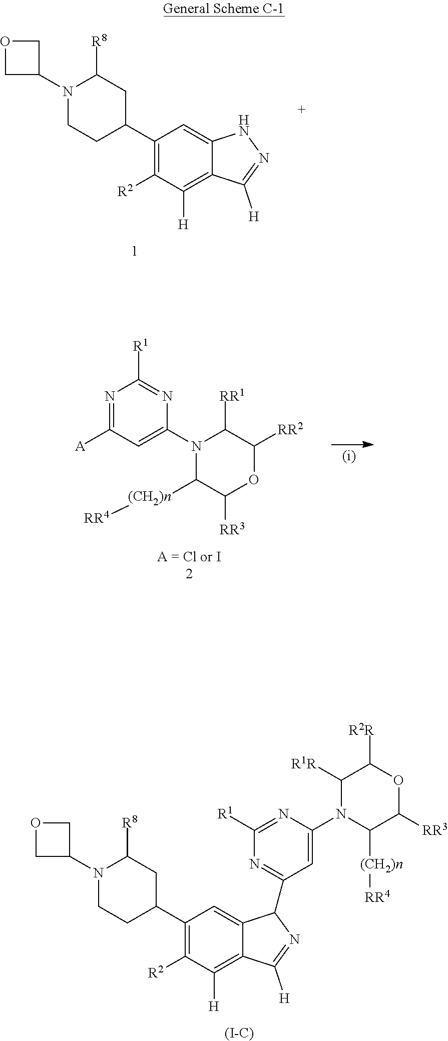

General Scheme C-1 provides an exemplary synthesis for preparing compounds of Formula (I-C). In Scheme C-1, R¹, R², RR¹, RR², RR³, RR⁴, R⁸ and n are as defined in Formula (I-C). Step (i) may be a substitution reaction by reacting compound 1 with compound 2 using appropriate base such as Cs₂CO₃ in an appropriate solvent such as N, N-dimethylformamide (DMF) under suitable temperature such as about 100° C. to provide a compound of Formula (I-C).

Step (i) may alternatively be a coupling reaction using appropriate reagents such as CuI and N,N′-dimethyl-cyclohexane-1,2-diamine in the presence of suitable base such as K₃PO₄ in a suitable solvent such as toluene at suitable temperature such as reflux condition to provide a compound of Formula (I-C).

Step (i) may alternatively be a coupling reaction using appropriate reagents such as Pd₂dba₃ and di-tert-butyl(2′,4′,6′-triisopropyl-[1,1′-biphenyl]-2-yl)phosphine in the presence of suitable base such as sodium tert-butoxide in a suitable solvent such as toluene at suitable temperature such as 100° C. to provide a compound of Formula (I-C).

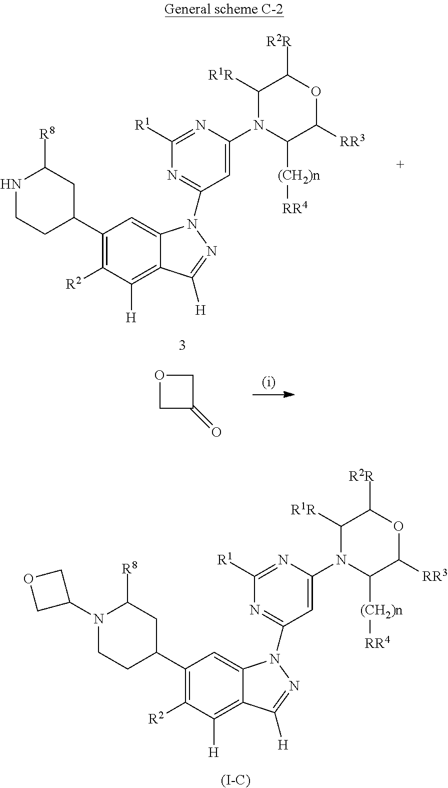

General Scheme C-2 provides an alternative exemplary synthesis for preparing compounds of Formula (I-C). In Scheme C-2, R¹, R², RR¹, RR², RR³, RR⁴, R⁸ and n are as defined in Formula (I-C).

Step (i) may be a coupling reaction using appropriate reagents in the presence of suitable base such as NaBH₃CN in the presence of a catalyst for example AcOH, in a suitable solvent such as DCM or DCE at suitable temperature such as room temperature to provide a compound of Formula (I-C).

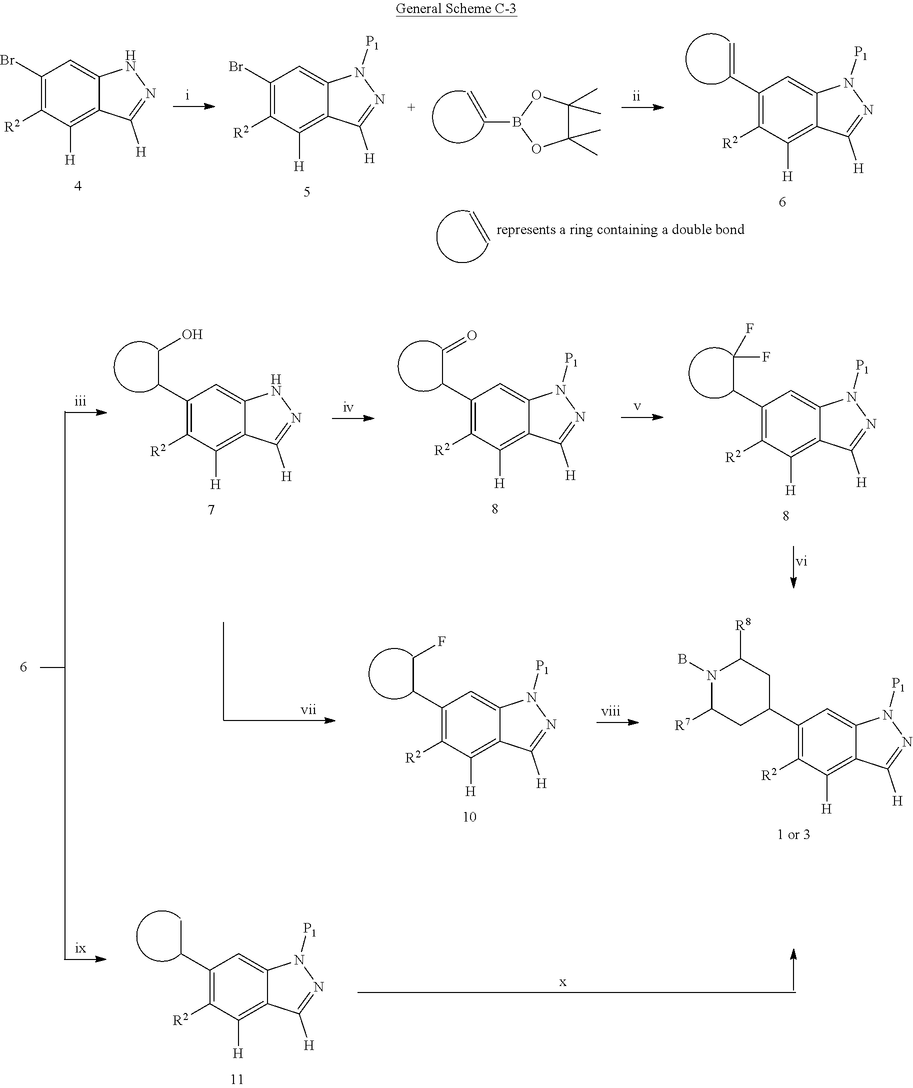

General Scheme C-3 provides an exemplary synthesis for preparing intermediate 1 or 3, wherein B is either H (for intermediate 3) or oxetanyl (for intermediate 1). The protecting group PG₁ can be any suitable protecting groups for example, tetrahydro-2H-pyran-2-yl (THP), (trimethylsilyl)ethoxy)methyl (SEM) or Acetyl (Ac).

Step (i) is a reaction with a suitable reagent such as DHP in the presence of a suitable acid such as TsOH in appropriate solvents such as DCM at a suitable temperature such as 20° C. to 40° C.

Step (ii) is a cross-coupling reaction with a suitable reagent such as boronic acid or esters using appropriate palladium catalysts such as Pd(dppf)Cl₂ in the presence of suitable bases such as Na₂CO₃ in appropriate solvents such as 1,4-dioxane under suitable temperatures such as 60° C. to 100° C.

Step (iii) is a reaction with a suitable oxidation reagent such as H₂O₂ in a suitable solvent such as THF under suitable temperatures such as −60° C. to −10° C. to provide intermediate 1 d.

Step (iv) is an oxidation reaction comprising reaction with an oxidant such as DMP in a suitable solvent such as DCM at a suitable temperature such as 0° C. to 25° C.

Steps (v) and (vii) involve reaction with a fluridizer such as DAST in suitable solvents such as DCM under suitable temperatures such as −78° C. to 0° C.

Step (ix) is a reaction with a suitable reducing reagent such as hydrogen in the presence of suitable catalysts such Pd/C in polar solvents such as MeOH at appropriate temperatures such as 25° C. to 80° C.

Steps (vi), (viii) and (x) are de-protection reactions. Typically, the intermediate is reacted with suitable acids such HCl in suitable solvents such as 1,4-dioxane under suitable temperatures such as 25° C. to 40° C. to give intermediate 1.

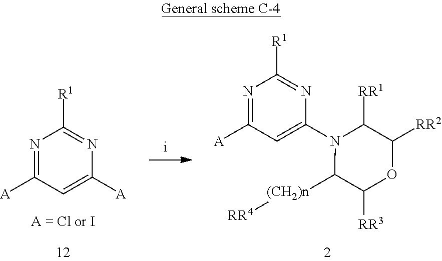

General Scheme C-4 provides an exemplary synthesis for preparing intermediate 2, wherein R¹, RR¹, RR², RR³ and RR⁴ are as defined in Formula (I-C).

Step (i) is a reaction between intermediate 12 with the appropriate amine using appropriate bases such as TEA in appropriate solvents such as EtOH at a suitable temperature such as 25° C. to 100° C.

Step (i) can alternatively be a coupling reaction with suitable reagents such as boronic acid in the presence of catalysts such as Pd(PPh₃)₂Cl₂ in suitable solvents such as 1,4-dioxane under 25° C. to 130° C.

### EXAMPLES

### General Experimental Procedures

The following descriptions and examples illustrate the invention. These examples are not intended to limit the scope of the present invention, but rather to provide guidance to the skilled chemist to prepare and use the compounds, compositions and methods of the present invention. While particular embodiments of the present invention are described, the skilled chemist will appreciate that various changes and modifications can be made without departing from the spirit and scope of the invention.

The chemical names of compounds described in the present application were generally created from ChemDraw Ultra (ChambridgeSoft) and/or generally follow the principle of IUPAC nomenclature.

Heating of reaction mixtures with microwave irradiations was carried out on a Smith Creator (purchased from Personal Chemistry, Forboro/MA, now owned by Biotage), an Emrys Optimizer (purchased from Personal Chemistry) or an Explorer (provided by CEM Discover, Matthews/NC) microwave.

Conventional techniques may be used herein for work up of reactions and purification of the products of the Examples.

References in the Examples below relating to the drying of organic layers or phases may refer to drying the solution over magnesium sulfate or sodium sulfate and filtering off the drying agent in accordance with conventional techniques. Products may generally be obtained by removing the solvent by evaporation under reduced pressure.

Purification of the compounds in the examples may be carried out by conventional methods such as chromatography and/or re-crystallization using suitable solvents. Chromatographic methods are known to the skilled person and include e.g. column chromatography, flash chromatography, HPLC (high performance liquid chromatography), and MDAP (mass directed auto-preparation, also referred to as mass directed LCMS purification). MDAP is described in e.g. W. Goetzinger et al, Int. J. Mass Spectrom., 2004, 238, 153-162.

Analtech Silica Gel GF and E. Merck Silica Gel 60 F-254 thin layer plates were used for thin layer chromatography. Both flash and gravity chromatography were carried out on E. Merck Kieselgel 60 (230-400 mesh) silica gel. Preparative HPLC were performed using a Gilson Preparative System using a Luna 5 u C18(2) 100A reverse phase column eluting with a 10-80 gradient (0.1% FA in acetonitrile/0.1% aqueous FA) or a 10-80 gradient (acetonitrile/water). The CombiFlash system used for purification in this application was purchased from Isco, Inc. CombiFlash purification was carried out using a pre-packed SiO₂ column, a detector with UV wavelength at 254 nm and mixed solvents.

The terms “CombiFlash”, “Biotage®”, “Biotage 75” and “Biotage SP4®” when used herein refer to commercially available automated purification systems using pre-packed silica gel cartridges.

Final compounds were characterized with LCMS (conditions listed below) or NMR. ¹H NMR or ¹⁹FNMR spectra were recorded using a Bruker Avance 400 MHz spectrometer. CDCl₃ is deuteriochloroform, DMSO-d₆ is hexadeuteriodimethylsulfoxide, and CD₃OD is tetradeuteriomethanol. Chemical shifts are reported in parts per million (ppm) downfield from the internal standard tetramethylsilane (TMS) or the NMR solvent. Abbreviations for NMR data are as follows: s=singlet, d=doublet, t=triplet, q=quartet, m=multiplet, dd=doublet of doublets, dt=doublet of triplets, app=apparent, br=broad. J indicates the NMR coupling constant measured in Hertz.

All temperatures are reported in degrees Celsius. All other abbreviations are as described in the ACS Style Guide (American Chemical Society, Washington, D.C., 1986).

Absolute stereochemistry can be determined by methods known to one skilled in the art, for example X-ray or Vibrational Circular Dichroism (VCD).

When an enantiomer or a diasteroisomer is described and the absolute stereochemistry of a chiral centre is not known, the use of “*” at the chiral centre denotes that the absolute stereochemistry of the chiral centre is not known, i.e. the compound as drawn may be either a single R enantiomer or a single S enantiomer. Where the absolute stereochemistry at a chiral centre of an enantiomer or a diasteroisomer is known, a bold wedge symbol ( [symbol] ) or a hashed wedge symbol ( [symbol] ) is used as appropriate, without the use of “*” at the chiral centre.

When a geometric or cis-trans isomer is described and the absolute configuration of the isomer is not known, the use of “*” at one of the atoms relevant to the geometric or cis-trans isomerism denotes that the absolute configuration at or around that atom is not known, i.e. the compound as drawn may be either a single cis isomer or a single trans enantiomer.

In the procedures that follow, after each starting material, reference to an intermediate is typically provided. This is provided merely for assistance to the skilled chemist. The starting material may not necessarily have been prepared from the batch referred to.

### LCMS Conditions:

1) Acidic method:
a. Instruments: HPLC: Waters UPC2 and MS: Qda
Mobile phase: water containing 0.1% FA/0.1% MeCN

### Column: ACQUITY UPLC BEH C₁₈ 1.7 μm 2.1×50 mm and 1.7 μm 2.1×100 mm

Detection: MS and photodiode array detector (PDA)
b. Instruments: HPLC: Shimadzu and MS: 2020
Mobile phase: water containing 0.1% FA/0.1% MeCN
Column: Sunfire C₁₈ 5 μm 50×4.6 mm and Sunfire C₁₈ 5 μm 150×4.6 mm
Detection: MS and photodiode array detector (PDA)
2) Basic conditions:

### Instruments: HPLC: Agilent 1260 and MS: 6120

Mobile phase: 0.1% NH₄OH in H₂O/0.1% NH₄OH in ACN
Column: Xbridge C₁₈ 5 μm 50×4.6 mm and Xbridge C₁₈ 5 μm 150×4.6 mm
Detection: MS and photodiode array detector (DAD)

### Prep-HPLC Conditions

Instrument: Waters instrument
Column: Xbridge Prep C₁₈ column OBD (10 μm, 19×250 mm), Xbrige prep C₁₈ 10 μm OBD™ 19×150 mm, Sunfire Prep C₁₈ 10×250 mm 5 μm, XBRIDGE Prep C₁₈ 10×150 mm 5 μm, etc

### Acidic Method:

Mobile phase: water containing 0.1% TFA/acetonitrile.

### Basic Method:

Mobile phase: water containing 0.1% NH₄OH/acetonitrile.

### Chiral Prep-HPLC:

Thar SFC Prep 80 (TharSFC ABPR1, TharSFC SFC Prep 80 CO₂ Pump, TharSFC Co-Solvent Pump, TharSFC Cooling Heat Exchanger and Circulating Bath, TharSFC Mass Flow Meter, TharSFC Static Mixer, TharSFC Injection Module, Gilson UV Detector, TharSFC Fraction Collection Module

### Chiral-HPLC Analysis:

Instrument: Thar SFC Prep 80 (TharSFC ABPR1, TharSFC SFC Prep 80 CO₂Pump, TharSFC Co-Solvent Pump, TharSFC Cooling Heat Exchanger and Circulating Bath, TharSFC Mass Flow Meter, TharSFC Static Mixer, TharSFC Injection Module, Gilson UV Detector, TharSFC Fraction Collection Module
Column and mobile phase: are described in below examples.

### Abbreviations and Resource Sources

The following abbreviations and resources are used herein below:

Ac—acetyl
MeCN—acetonitrile
Atm—atmosphere
Aq.—aqueous
BINAP—2,2′-bis(diphenylphosphino)-1,1′-binaphthyl
Boc—tert-butyloxycarbonyl
Boc₂O—di-tert-butyl dicarbonate
Bn—benzyl
t-Bu—tert-butyl
conc.—concentrated
DAST— N,N-diethylaminosulfur trifluoride
DCE— 1,2-dichloroethane
DCM—dichloromethane
DEA— diethanolamine

### DMEDA—N,N′-Dimethylethylenediamine

Dess-Martin—1,1,1-Tris(acetyloxy)-1,1-dihydro-1,2-benziodoxol-3-(1H)-one
DHP—3,4-dihydro-2H-pyran
DIBAL-H—diisobutylaluminum hydride

### DIEA—N,N-diisopropylethylamine

### DIPEA—N, N-diisopropylethylamine

### DMA—N, N-dimethylacetamide

DMAP—4-dimethylaminopyridine
DMEDA—N,N′-dimethylethylenediamine

### DMF—N, N-dimethylformamide

DMP—Dess-Martin periodinane
DMSO—dimethyl sulfoxide
DPPF—1,1′-bis(diphenylphosphino)ferrocene
EA—ethyl acetate
EDC—1-ethyl-3-(3-dimethylaminopropyl)carbodiimide hydrochloride
EDCI—3-(ethyliminomethyleneamino)-N,N-dimethylpropan-1-amine
EtOH/EtOH—ethanol
Et₂O—diethyl ether
EtOAc—ethyl acetate
Et₃N—triethylamine
FA—formic acid
HEP—heptane
Hex—hexane
HOAc—acetic acid
HATU—2-(1H-7-azabenzotriazol-1-yl)-1,1,3,3-tetramethyl uranium hexafluorophosphate
HOBT—hydroxybenzotriazole
IPA—isopropyl alcohol
ⁱPrOH/iPrOH—isopropyl alcohol
m-CPBA—meta-chloroperoxybenzoic acid
MOMCl—monochlorodimethyl ether
Me—methyl
MeOH—methanol
MsCl—methanesulfonyl chloride
NaHMDS—sodium bis(trimethylsilyl)amide

### NIS—N-iodosuccinimide

NMP—1-methyl-2-pyrrolidone
NMO—4-methylmorpholine 4-oxide
PE—petroleum ether
PMB—p-methoxybenzyl
Pd₂(dba)₃—Tris(dibenzylideneacetone)dipalladium
Pd(dppf)Cl₂—1,1′-Bis(diphenylphosphino)ferrocenepalladium(II)dichloride dichloromethane complex
Ph₃P—triphenylphosphine

### PhNTf₂—N,N-bis-(Trifluoromethanesulfonyl)aniline

PPTS—pyridinium p-toluenesulfonate
PTSA—p-toluenesulfonic acid
rt/RT—room temperature
Rt—retention time
sat.—saturated
SEM-Cl—2-(trimethylsilyl)ethoxymethyl chloride

### SFC—Supercritical Fluid Chromatography

TBAI—Tetrabutylammonium iodide
TBDPSCl—tert-Butyl(chloro)diphenylsilane
TEA—triethylamine
TFA—trifluoroacetic acid
TFAA—trifluoroacetic anhydride
THF—tetrahydrofuran
TLC—thin layer chromatography
TsCl—4-toluenesulfonyl chloride
TsOH—p-toluenesulfonic acid

### Description A-1

### (S)-Morpholin-2-ylmethanol hydrochloride (D A-1)

To a solution of (S)-tert-butyl 2-(hydroxymethyl)morpholine-4-carboxylate (500 mg, 2.30 mmol) in dioxane (4 mL) was added HCl/dioxane (4 M, 5 mL) and stirred at rt for 2 hrs. TLC showed that the reaction was completed. The reaction mixture was concent-rated to give the title compound (crude, 430 mg, yield >100%) as a white solid.

### Description A-2

### 4,6-Diiodo-2-methylpyrimidine (D A-2)

To a solution of NaI (11.9 g, 79.7 mmol) in HI (55%, 50 mL) was added 4,6-dichloro-2-methylpyrimidine (10.0 g, 61.3 mmol) in portions. The resulting suspension was heated to 40° C. and stirred for 1 hour. The reaction mixture was cooled and filtered. The solid was washed with water and then washed with methanol (50 mL). The mixture was filtered to give the title compound (9.0 g, yield 42%) as a white solid.

¹H NMR (400 MHz, CDCl₃): δ 8.07 (s, 1H), 2.67 (s, 3H).

LCMS (mobile phase: 5-95% acetonitrile in 2.5 min): Rt=1.59 min, MS Calcd: 346; MS Found: 347 [M+H]⁺.

### Description A-3

### (S)-(4-(6-Iodo-2-methylpyrimidin-4-yl)morpholin-2-yl)methanol (D A-3)

To a solution of (S)-morpholin-2-ylmethanol hydrochloride (430 mg crude, 2.80 mmol) in CH₃OH (5 mL) was added 4,6-diiodo-2-methylpyrimidine (1.10 g, 3.10 mmol) and TEA (850 mg, 8.40 mmol). The resulting mixture was warmed to 60° C. for 2 hrs. TLC showed the reaction was completed. The reaction mixture was diluted with water (20 mL) and extracted EtOAc (20 mL×2). The combined organic layers were concentrated. The crude was purified by gel silico column (PE:EA=5:1) to give the title compound (760 mg, yield 81%) as a white solid.

¹H NMR (300 MHz, CDCl₃): δ 6.79 (s, 1H), 4.18-4.01 (m, 3H), 3.79-3.58 (m, 4H), 3.08-2.99 (m, 1H), 2.92-2.84 (m, 1H), 2.46 (s, 3H), 1.97-1.90 (m, 1H).

### Description A-4

### 6-Bromo-5-methyl-1H-indazole (D A-4)

To a solution of 5-bromo-2,4-dimethylaniline (15.0 g, 75.0 mmol) in chloroform (150 mL) were added Ac₂O (15.0, 150 mmol), KOAc (8.00 g, 82.5 mmol), 18-crown-6 (10.0 g, 37.5 mmol) and isoamyl nitrite (26.3 g, 225 mmol) under ice bath. The reaction mixture was refluxed for 36 hrs, then concentrated to remove solvent. The residue was dissolved in EtOAc (500 mL), washed with water (100 mL), dried over Na₂SO₄, filtered and concentrated. The residue was dissolved in THF (100 mL) and NaOH (4 M, 40.0 mL, 160 mmol) was added. The mixture was stirred at rt for 1 h. The solvent was removed under vacuum and the residue was partitioned between EtOAc (400 mL) and water (200 mL). The organic layer was washed with brine, dried over Na₂SO₄, filtered and concentrated. The crude was purified by column chromatography (PE:EtOAc from 10:1 to 5:1) to give the title compound (5.1 g, yield 32%) as an orange solid.

¹H NMR (300 MHz, CDCl₃): δ 10.20 (br s, 1H), 7.99 (s, 1H), 7.75 (s, 1H), 7.61 (s, 1H), 2.50 (s, 3H).

### Description A-5

### 6-Bromo-5-methyl-1-(tetrahydro-2H-pyran-2-yl)-1H-indazole (D A-5)

To a solution of 6-bromo-5-methyl-1H-indazole (5.10 g, 24.2 mmol) in dry DCM (120 mL) was added DHP (4.10 g, 48.4 mmol), TsOH (0.800 g, 4.80 mmol) and Mg₂SO₄ (5.0 g) at rt. The reaction mixture was heated to 35° C. and stirred for an hour. The reaction mixture was filtered and the filtrate was washed with a solution of Na₂CO₃ (10%, 100 mL), dried over Na₂SO₄, filtered and concentrated. The crude was purified by column chromatography (PE: EtOAc=50/1 to 20/1) to give the title compound (6.0 g, yield 84%) as an orange solid.

¹H NMR (300 MHz, CDCl₃): δ 7.90 (s, 1H), 7.84 (s, 1H), 7.55 (s, 1H), 5.63 (dd, J=9.6, 3.0 Hz, 1H), 4.05-4.00 (m, 1H), 3.78-3.70 (m, 1H), 2.58-2.44 (m, 4H), 2.20-2.02 (m, 2H), 1.78-1.65 (m, 3H).

LCMS (mobile phase: 5-95% CH₃CN): Rt=2.19 min in 3 min; MS Calcd: 294; MS Found: 295 [M+H]⁺.

### Description A-6

### tert-Butyl 4-(5-methyl-1-(tetrahydro-2H-pyran-2-yl)-1H-indazol-6-yl)-5,6-dihydropyridine-1(2H)-carboxylate (D A-6)

To a suspension of 6-bromo-5-methyl-1-(tetrahydro-2H-pyran-2-yl)-1H-indazole (5.50 g, 18.6 mmol), tert-butyl 4-(4,4,5,5-tetramethyl-1,3,2-dioxaborolan-2-yl)-5,6-dihydropyridine-1(2H)-carboxylate (6.90 g, 22.3 mmol) and Na₂CO₃ (4.90 g, 46.5 mmol) in dioxane (150 mL) and water (130 mL) was added Pd(dppf)Cl₂ (658 mg, 0.900 mmol). The mixture was degassed with N₂ for 3 times and then stirred at 80° C. overnight. The solvent was removed under vacuum and the residue was partitioned between EtOAc (300 mL) and water (200 mL). The separated organic layer was washed with brine, dried over Na₂SO₄, filtered and concentrated. The crude was purified by column chromatography (PE:EtOAc=10:1) to give the title compound (7.3 g, yield 99%) as a slight brown solid.

¹H NMR (400 MHz, CDCl₃): δ 7.92 (s, 1H), 7.48 (s, 1H), 7.28 (s, 1H), 5.67 (dd, J=9.6, 2.8 Hz, 1H), 5.63 (br s, 1H), 4.07-4.01 (m, 3H), 3.78-3.70 (m, 1H), 3.67-3.64 (m, 2H), 2.62-2.53 (m, 1H), 2.45-2.39 (m, 2H), 2.34 (s, 3H), 2.18-2.12 (m, 1H), 2.07-2.02 (m, 1H), 1.81-1.73 (m, 2H), 1.69-1.61 (m, 1H), 1.52 (s, 9H).

### Description A-7

### tert-Butyl 4-(5-methyl-1-(tetrahydro-2H-pyran-2-yl)-1H-indazol-6-yl)piperidine-1-carboxylate (D A-7)

To a solution of tert-butyl 4-(5-methyl-1-(tetrahydro-2H-pyran-2-yl)-1H-indazol-6-yl)-5,6-dihydropyridine-1(2H)-carboxylate (80 g, crude) in MeOH (2 L) under H₂ was added Pd/C (10 g, 12%/W). The reaction mixture was degassed for 3 times, stirred at r.t for 2 d, filtered and concentrated to give the crude product as a white solid. (65.8 g)

LC-MS [mobile phase: mobile phase: from 30% water (0.1% FA) and 70% CH₃CN (0.1% FA) to 5% water (0.1% FA) and 95% CH₃CN (0.1% FA) in 2.0 min]: Rt=0.63 min; MS Calcd.: 399.2, MS Found: 400.5 [M+H]⁺.

### Description A-8

### 5-Methyl-6-(piperidin-4-yl)-1H-indazole (D A-8)

To a solution of tert-butyl 4-(5-methyl-1-(tetrahydro-2H-pyran-2-yl)-1H-indazol-6-yl)piperidine-1-carboxylate (55.4 g, 139 mmol) in MeOH (150 mL) was added HCl/MeOH (5 M, 200 mL). The reaction mixture was stirred at rt overnight, then concentrated, treated with Na₂CO₃ aq. and basified with NaOH aq. to pH >12. The mixture was filtered to give the desired product as a white solid. (29.3 g, yield=98%)

LC-MS [mobile phase: mobile phase: from 90% water (0.1% FA) and 10% CH₃CN (0.1% FA) to 5% water (0.1% FA) and 95% CH₃CN (0.1% FA) in 2.0 min]: Rt=0.85 min; MS Calcd.: 215, MS Found: 216 [M+H]⁺.

### Description A-9

### 3-((Phenylsulfonyl)methylene)oxetane (D A-9)

To a solution of (methylsulfonyl)benzene (2.2 g, 13.9 mmol) in THF (38 mL) at 0° C. was added n-BuLi (2.5 M in hexanes, 12.2 mL, 30.6 mmol) dropwise over 10 minutes. After the mixture was stirred for 30 min, chlorodiethylphosphonate (2.4 mL, 16.7 mmol) was added dropwise to the reaction. After 30 minutes, a solution of oxetan-3-one (1.0 g, 13.9 mmol) in THF (2 mL) was added dropwise to the reaction mixture at −78° C. The reaction mixture was stirred at −78° C. for 2 hours, then diluted with aqueous NH₄Cl (100 mL) and extracted with EtOAc (100 mL×2). The combined organic layers were concentrated and the residue was purified by silica gel chromatxography column (petroleum ether/EtOAc=3/1) to give the title compound (2.4 g, 82%) as a colorless oil.

¹H NMR (400 MHz, CDCl₃): δ 7.90-7.88 (m, 2H), 7.68-7.64 (m, 1H), 7.57 (t, J=7.6 Hz, 2H), 6.13-6.11 (m, 1H), 5.66-5.64 (m, 2H), 5.30-5.27 (m, 2H).

### Description A-10

### 5-Methyl-6-(1-(3-((phenylsulfonyl)methyl)oxetan-3-yl)piperidin-4-yl)-1H-indazole (D A-10)

To a stirred solution of 3-((phenylsulfonyl)methylene)oxetane (630 mg, 2.99 mmol) in MeOH (5 ml) was added 5-methyl-6-(piperidin-4-yl)-1H-indazole (500 mg, 2.32 mmol). The reaction mixture was stirred at 50° C. overnight, then concentrated. The purification via column chromatography afforded the desired product as a white solid (816 mg, yield: 82%).

LC-MS [mobile phase: from 90% water (0.1% FA) and 10% CH₃CN (0.1% FA) to 5% water (0.1% FA) and 95% CH₃CN (0.1% FA) in 2.0 min]: Rt=1.31 min; MS Calcd: 425, MS Found: 426 [M+H]⁺.

### Description A-11

### 5-Methyl-6-(1-(3-methyloxetan-3-yl)piperidin-4-yl)-1H-indazole (D A-11)

To a stirred solution of 5-methyl-6-(1-(3-((phenylsulfonyl)methyl)oxetan-3-yl)piperidin-4-yl)-1H-indazole (400 mg, 0.940 mmol) in MeOH/THF (12 ml/2.4 ml) was added Mg (114 mg, 4.70 mmol). The reaction mixture was stirred at room temperature overnight. Another portion of Mg (152 mg, 6.27 mmol) was added. The reaction mixture was stirred at 40° C. overnight, then cooled to room temperature, diluted with Et₂O, treated with Na₂SO₄′10H₂O, stirred for an hour and filtered. The filtrate was concentrated and purified by column chromatography (eluent: PE:EtOAc=1:1, followed by CH₂Cl₂:MeOH=30:1 to 15:1) afforded the desired product as a white solid (114 mg, yield: 42%).

LC-MS [mobile phase: from 90% water (0.1% FA) and 10% CH₃CN (0.1% FA) to 5% water (0.1% FA) and 95% CH₃CN (0.1% FA) in 2.0 min]: Rt=0.92 min; MS Calcd: 285, MS Found: 286 [M+H]⁺.

### Description A-12

### (R)-Morpholin-2-ylmethanol hydrochloride (D A-12)

To a solution of (R)-tert-butyl 2-(hydroxymethyl)morpholine-4-carboxylate (500 mg, 2.30 mmol) was added HCl/dioxane (4 M, 10 mL) and stirred for 1 h at rt. TLC showed that the reaction was completed. The reaction was concentrated to give the title compound (420 mg, yield >100%) as a white solid.

¹H NMR (300 MHz, DMSO-d): δ 9.67 (s, 1H), 9.38 (s, 1H), 3.94-3.88 (m, 1H), 3.77-3.67 (m, 2H), 3.45-3.33 (m, 2H), 3.13 (t, J=12.6 Hz, 2H), 2.95-2.87 (m, 1H), 2.78-2.67 (m, 1H).

### Description A-13

### (R)-(4-(6-Iodo-2-methylpyrimidin-4-yl)morpholin-2-yl)methanol (D A-13)

To a solution of (R)-morpholin-2-ylmethanol hydrochloride (423 mg crude, 2.30 mmol) in CH₃OH (10 mL) was added 4,6-diiodo-2-methylpyrimidine (954 mg, 2.75 mmol) and TEA (835 mg, 8.25 mmol). The resulting mixture was warmed to 70° C. and stirred for 2 hrs. LCMS showed that the reaction was completed. The reaction mixture was concentrated to remove solvent, poured into water (40 mL) and extracted with EtOAc (40 mL×2). The combined organic layers were washed with brine, dried over Na₂SO₄ and concentrated. The residue was purified by column (PE:EA=2:1) to give the title compound (639 mg, yield 83%) as a white solid.

¹H NMR (300 MHz, CDCl₃): δ 6.79 (s, 1H), 4.22-4.01 (m, 3H), 3.79-3.56 (m, 4H), 3.08-2.98 (m, 1H), 2.88-2.84 (m, 1H), 2.46 (s, 3H), 2.09-2.04 (m, 1H).

### Description A-14

### 4,6-Diiodo-2-methoxypyrimidine (D A-14)

To a solution of NaI (1.10 g, 7.34 mmol) in HI (55%, 7.5 mL) was added 4,6-dichloro-2-methoxypyrimidine (1.00 g, 5.59 mmol). The reaction mixture was heated to 40° C. and stirred for 10 h, then poured into ice water (50 mL) and filtered to give the crude solid. The residue was purified by column chromatography (PE:EtOAc=10:1) to give the title product (640 mg, yield 31.7%) as a white solid.

¹H NMR (400 MHz, CDCl₃): δ 7.85 (s, 1H), 4.00 (s, 3H).

### Description A-15

### (R)-(4-(6-Iodo-2-methoxypyrimidin-4-yl)morpholin-2-yl)methanol (D A-15)

The title compound was prepared by a procedure similar to those described for D A-3 starting from a solution of 4,6-diiodo-2-methoxypyrimidine and (R)-morpholin-2-ylmethanol hydrochloride in ⁱPrOH and DIPEA.

LC-MS [mobile phase: from 50% water (0.1% FA) and 50% CH₃CN (0.1% FA) to 5% water (0.1% FA) and 95% CH₃CN (0.1% FA) in 2.6 min]: Rt=0.92 min; MS Calcd: 351.1, MS Found: 352.0 [M+H]⁺.

### Description A-16

### (S)-(4-(6-Iodo-2-methoxypyrimidin-4-yl)morpholin-2-yl)methanol (D A-16)

The title compound was prepared by a procedure similar to those described for D A-3 starting from a solution of 4,6-diiodo-2-methoxypyrimidine and (S)-morpholin-2-ylmethanol hydrochloride in iPrOH and DIPEA.

LC-MS [mobile phase: from 50% water (0.1% FA) and 50% CH₃CN (0.1% FA) to 5% water (0.1% FA) and 95% CH₃CN (0.1% FA) in 2.6 min]: Rt=0.91 min; MS Calcd: 351.1, MS Found: 352.0 [M+H]⁺.

### Description B-1

### 6-Bromo-5-methyl-1H-indazole (D B-1)

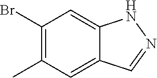

To a solution of 5-bromo-2,4-dimethylaniline (15.0 g, 75.0 mmol) in chloroform (150 mL) was added Ac₂O (15.0, 150 mmol) under ice bath. KOAc (8.00 g, 82.5 mmol), 18-crown-6 (10.0 g, 37.5 mmol) and isoamyl nitrite (26.3 g, 225 mmol) were added. The mixture was refluxed for 36 hrs. The reaction mixture was concentrated and the residue was dissolved in EtOAc (500 mL). The organic solution was washed with water (100 mL), dried over Na₂SO₄ and concentrated. The residue was dissolved in THF (100 mL) and NaOH (4 M, 40.0 mL, 160 mmol) was added. The mixture was stirred at rt for 1 h. The solvent was removed under vacuum and the residue was partitioned between EtOAc (400 mL) and water (200 mL). The organic layer was washed with brine, dried over Na₂SO₄ and concentrated. The crude was purified by column chromatography (PE:EtOAc from 10:1 to 5:1) to give the title compound (5.1 g, yield 32%) as an orange solid.

¹H NMR (300 MHz, CDCl₃): δ 10.20 (br, 1H), 7.99 (s, 1H), 7.75 (s, 1H), 7.61 (s, 1H), 2.50 (s, 3H).

### Description B-2

### 6-Bromo-5-methyl-1-(tetrahydro-2H-pyran-2-yl)-1H-indazole (D B-2)

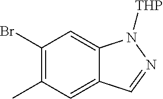

To a solution of 6-bromo-5-methyl-1H-indazole (5.10 g, 24.2 mmol) in dry DCM (120 mL) was added DHP (4.10 g, 48.4 mmol), TsOH (0.800 g, 4.80 mmol) and Mg₂SO₄ (5.0 g) at rt. The reaction mixture was heated to 35° C. and stirred for an hour. The reaction mixture was filtered and the filtrate was washed with a solution of Na₂CO₃ (10%, 100 mL), dried over Na₂SO₄ and concentrated. The crude was purified by column chromatography (PE:EtOAc from 50:1 to 20:1) to give the title compound (6.0 g, yield 84%) as an orange solid.

¹H NMR (300 MHz, CDCl₃): δ 7.90 (s, 1H), 7.84 (s, 1H), 7.55 (s, 1H), 5.63 (dd, J=9.6, 3.0 Hz, 1H), 4.05-4.00 (m, 1H), 3.78-3.70 (m, 1H), 2.58-2.44 (m, 4H), 2.20-2.02 (m, 2H), 1.78-1.65 (m, 3H).

LCMS (mobile phase: 5-95% CH₃CN): Rt=2.19 min in 3 min; MS Calcd: 294; MS Found: 295 [M+H]⁺.

### Description B-3

### Tert-butyl 4-(5-methyl-1-(tetrahydro-2H-pyran-2-yl)-1H-indazol-6-yl)-5,6-dihydropyridine-1(2H)-carboxylate (D B-3)

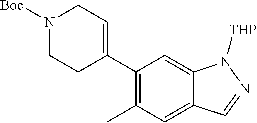

To a suspension of 6-bromo-5-methyl-1-(tetrahydro-2H-pyran-2-yl)-1H-indazole (5.50 g, 18.6 mmol), tert-butyl 4-(4,4,5,5-tetramethyl-1,3,2-dioxaborolan-2-yl)-5,6-dihydropyridine-1(2H)-carboxylate (6.90 g, 22.3 mmol) and Na₂CO₃ (4.90 g, 46.5 mmol) in dioxane (150 mL) and water (130 mL) was added Pd(dppf)Cl₂ (658 mg, 0.900 mmol). The mixture was degassed with N₂ for 3 times and then stirred at 80° C. overnight. The solvent was removed under vacuum and the residue was partitioned between EtOAc (300 mL) and water (200 mL). The combined organic layers were washed with brine, dried over Na₂SO₄ and concentrated. The crude was purified by column chromatography (PE:EtOAc=10:1) to give the title compound (7.3 g, yield 99%) as a slight brown solid.

¹H NMR (400 MHz, CDCl₃): δ 7.92 (s, 1H), 7.48 (s, 1H), 7.28 (s, 1H), 5.67 (dd, J=9.6, 2.8 Hz, 1H), 5.63 (br s, 1H), 4.07-4.01 (m, 3H), 3.78-3.70 (m, 1H), 3.67-3.64 (m, 2H), 2.62-2.53 (m, 1H), 2.45-2.39 (m, 2H), 2.34 (s, 3H), 2.18-2.12 (m, 1H), 2.07-2.02 (m, 1H), 1.81-1.73 (m, 2H), 1.69-1.61 (m, 1H), 1.52 (s, 9H).

### Description B-4

### tert-butyl 4-(5-methyl-1-(tetrahydro-2H-pyran-2-yl)-1H-indazol-6-yl)piperidine-1-carboxylate (D B-4)

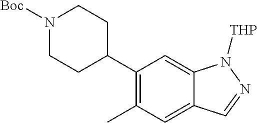

To a solution of tert-butyl 4-(5-methyl-1-(tetrahydro-2H-pyran-2-yl)-1H-indazol-6-yl)-5,6-dihydropyridine-1(2H)-carboxylate (80 g, crude) in MeOH (2 L) under H₂ was added Pd/C (10 g, 12% NV). The reaction mixture was degassed for 3 times and stirred at rt for 2 days. The mixture was filtered and the filtrate was concentrated to give the crude product as a white solid (65.8 g).

LC-MS [mobile phase: from 30% water (0.1% FA) and 70% CH₃CN (0.1% FA) to 5% water (0.1% FA) and 95% CH₃CN (0.1% FA) in 2.0 min]: Rt=0.63 min; MS Calcd.: 399.2, MS Found: 400.5 [M+H]⁺.

### Description B-5

### 5-methyl-6-(piperidin-4-yl)-1H-indazole (D B-5)

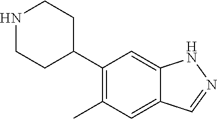 [SMILES: Cc1cc2cn[nH]c2cc1C1CCNCC1]

HCl/MeOH (5M, 200 mL) was added to a solution of tert-butyl 4-(5-methyl-1-(tetrahydro-2H-pyran-2-yl)-1H-indazol-6-yl)piperidine-1-carboxylate (55.4 g, 139 mmol) in MeOH (150 mL). The reaction mixture was stirred at rt overnight, then concentrated, treated with Na₂CO₃ aq. and basified with NaOH aq. to pH >12. The mixture was filtered to give the desired product as a white solid. (29.3 g, yield=98%)

LC-MS [mobile phase: mobile phase: from 90% water (0.1% FA) and 10% CH₃CN (0.1% FA) to 5% water (0.1% FA) and 95% CH₃CN (0.1% FA) in 2.0 min]: Rt=0.85 min; MS Calcd.: 215, MS Found: 216 [M+H]⁺.

### Description B-6

### 5-methyl-6-(1-(oxetan-3-yl)piperidin-4-yl)-1H-indazole (D B-6)

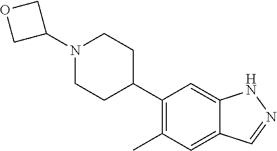 [SMILES: Cc1cc2cn[nH]c2cc1C1CCN(C2COC2)CC1]

NaBH₃CN (9.40 g, 149 mmol) was added to a solution of 5-methyl-6-(piperidin-4-yl)-1H-indazole (16.0 g, 74.3 mmol), oxetan-3-one (13.4 g, 223 mmol), zeolite (13.4 g) and AcOH (1.56 g, 1.63 mmol) in CH₂Cl₂/MeOH (320 mL/80 mL) at rt. The reaction mixture was stirred at rt overnight, filtered and the filtered cake was washed with CH₂Cl₂. The filtrate was washed with NaHCO₃ aq and brine. The organic part was dried over anhydrous Na₂SO₄, filtered and concentrated. The residue was purified by column (PE:EtOAc=1:1 to CH₂Cl₂:MeOH=50:1) to give the desired product as a white solid (11.9 g, yield=59%) LC-MS [mobile phase: from 90% water (0.1% FA) and 10% CH₃CN (0.1% FA) to 5% water (0.1% FA) and 95% CH₃CN (0.1% FA) in 2.0 min]: Rt=0.87 min; MS Calcd.: 271, MS Found: 272 [M+H]⁺.

### Descriptions B-7 and B-8

### (4-(6-iodo-2-methylpyrimidin-4-yl)-6-methylmorpholin-2-yl)methanol (Isomer 1, D B-7) and (4-(6-iodo-2-methylpyrimidin-4-yl)-6-methylmorpholin-2-yl)methanol (Isomer 2, D B-8)

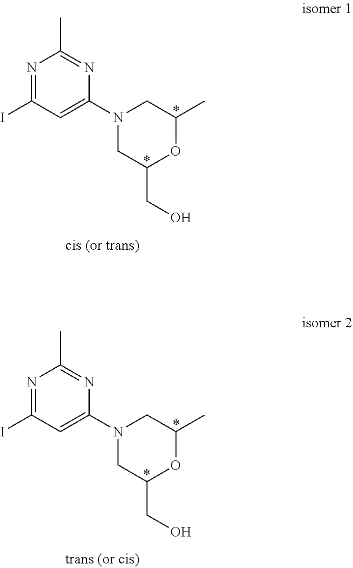 [SMILES: Cc1nc(I)cc(N2CC(C)OC(CO)C2)n1 ; Cc1nc(I)cc(N2CC(C)OC(CO)C2)n1]

DIPEA (886 mg, 6.90 mmol) was added to a solution of 4,6-diiodo-2-methylpyrimidine (792 mg, 2.29 mmol) and (6-methylmorpholin-2-yl)methanol (300 mg, 2.29 mmol) in THF/EtOH (7 mL/7 mL). The reaction mixture was stirred at room temperature overnight, then concentrated to give the residue. The residue was purified by silica gel chromatography (eluent: PE:EtOAc=5:1) afforded isomer 1 as a white solid (207 mg, yield: 25%) and isomer 2 as a white solid (172 mg, yield: 21%).

### Isomer 1:

LC-MS [mobile phase: from 80% water (0.1% FA) and 20% CH₃CN (0.1% FA) to 5% water (0.1% FA) and 95% CH₃CN (0.1% FA) in 10.0 min]: Rt=1.51 min; MS Calcd: 349, MS Found: 350 [M+H]⁺.

### Isomer 2:

LC-MS [mobile phase: from 80% water (0.1% FA) and 20% CH₃CN (0.1% FA) to 5% water (0.1% FA) and 95% CH₃CN (0.1% FA) in 10.0 min]: Rt=1.23 min; MS Calcd: 349, MS Found: 350 [M+H]⁺.

### Descriptions B-9 and B-10

### (4-(6-iodo-2-methylpyrimidin-4-yl)-5-methylmorpholin-2-yl)methanol (Single Unknown Isomer 1, D B-9; Single Unknown Isomer 2, D B-10)

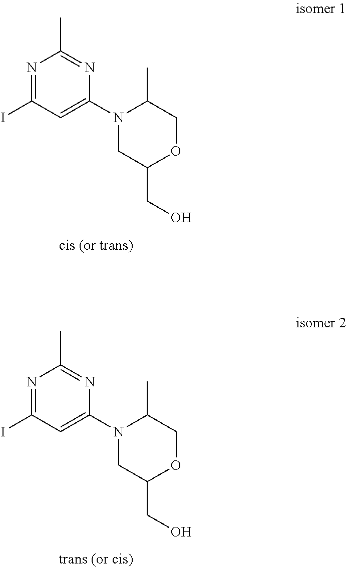 [SMILES: Cc1nc(I)cc(N2CC(CO)OCC2C)n1 ; Cc1nc(I)cc(N2CC(CO)OCC2C)n1]

To a solution of 4,6-diiodo-2-methylpyrimidine (792 mg, 2.29 mmol) and (6-methylmorpholin-3-yl)methanol (300 mg, 2.3 mmol) in i-PrOH (10 mL) was added DIPEA (886 mg, 6.9 mmol). The reaction mixture was stirred at 90° C. overnight, then concentrated to give the residue. The purification via silica gel chromatography (eluent: PE:EtOAc=5:1) afforded isomer 1 as a yellow solid (371 mg, yield: 46%) and isomer 2 as a bright oil (80 mg, yield: 21%).

### Isomer 1:

LC-MS [mobile phase: from 95% water (0.1% FA) and 5% CH₃CN (0.1% FA) to 5% water (0.1% FA) and 95% CH₃CN (0.1% FA) in 10.0 min]: Rt=3.76 min; MS Calcd: 349, MS Found: 350 [M+H]⁺.

### Isomer 2:

LC-MS [mobile phase: from 95% water (0.1% FA) and 5% CH₃CN (0.1% FA) to 5% water (0.1% FA) and 95% CH₃CN (0.1% FA) in 10.0 min]: Rt=3.66 min; MS Calcd: 34, MS Found: 350 [M+H]⁺.

### Description B-11

### 2-(4-(6-iodo-2-methylpyrimidin-4-yl)morpholin-2-yl)ethanol (D B-11)

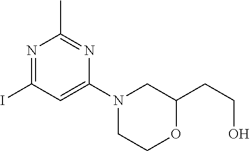 [SMILES: Cc1nc(I)cc(N2CCOC(CCO)C2)n1]

To a solution of 4,6-diiodo-2-methylpyrimidine (500 mg, 1.4 mmol) and 3-oxa-1,8-diazaspiro[4.5]decan-2-one (220 mg, 1.3 mmol) in EtOH/THF (7 mL/7 mL) was added DIPEA (508 mg, 3.9 mmol). The reaction mixture was stirred at rt overnight. The solvent was removed and the residue was purified by silica gel chromatography (PE:EtOAc=5:1) to give the desired product as a white solid (371 mg, yield: 81%).

LC-MS [mobile phase: from 70% water (0.1% FA) and 30% CH₃CN (0.1% FA) to 5% water (0.1% FA) and 95% CH₃CN (0.1% FA) in 2.0 min]: Rt=0.29 min; MS Calcd: 349.0, MS Found: 350.2 [M+H]⁺.

### Description B-12

### (4-(6-iodo-2-methoxypyrimidin-4-yl)-5-methylmorpholin-2-yl)methanol (D B-12)

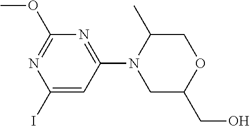 [SMILES: COc1nc(I)cc(N2CC(CO)OCC2C)n1]

To a solution of 4,6-diiodo-2-methoxypyrimidine (300 mg, 0.8 mmol) and (5-methylmorpholin-2-yl)methanol (109 mg, 0.8 mmol) in THF/EtOH=1/1 (30 mL) was added DIEA (320 mg, 2.5 mmol) at rt. The reaction was stirred at 60° C. for 48 h. The reaction mixture was concentrated and the residue was purified by silica gel chromatography eluted with PE:EtOAc=1:2 to afford product as a white solid (253 mg).

LC-MS [mobile phase: from 50% water (0.1% FA) and 50% CH₃CN (0.1% FA) to 5% water (0.1% FA) and 95% CH₃CN (0.1% FA) in 2.6 min]: Rt=1.01 min; MS Calcd: 365, MS Found: 366 [M+H]⁺.

### Description B-13

### tert-butyl 4-(1-(6-(2-(hydroxymethyl)-5-methylmorpholino)-2-methoxypyrimidin-4-yl)-5-methyl-1H-indazol-6-yl)piperidine-1-carboxylate (D B-13)

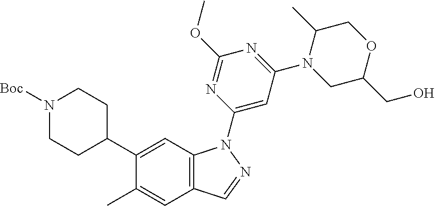 [SMILES: COc1nc(N2CC(CO)OCC2C)cc(-n2ncc3cc(C)c(C4CCN(C(=O)OC(C)(C)C)CC4)cc32)n1]

To a solution of tert-butyl 4-(5-methyl-1H-indazol-6-yl)piperidine-1-carboxylate (500 mg, 1.58 mmol) and (4-(6-iodo-2-methoxypyrimidin-4-yl)-5-methylmorpholin-2-yl)methanol (637 mg, 1.75 mmol), N,N′-dimethylcyclohexane-1,2-diamine (224 mg, 1.58 mmol), CuI (150 mg, 0.79 mmol) and K₃PO₄ (670 mg, 3.16 mmol) in toluene (10 mL) was stirred at 100° C. for 6 hours. The mixture was concentrated. The residue was purified by silica gel chromatography column (petroleum ether/EtOAc=1/1) to give the title compound (507 mg, yield given) as a white solid.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 05-95-POS; flow rate: 1.5 ml/min]: Rt=2.236 min; MS Calcd.: 552, MS Found: 553 [M+H]⁺.

### Description B-14

### methyl 3-(benzylamino)-2-hydroxybutanoate (D B-14)

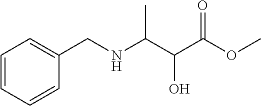 [SMILES: COC(=O)C(O)C(C)NCc1ccccc1]

A solution of methyl 3-amino-2-hydroxybutanoate hydrochloride (25.0 g, 147 mmol) in MeOH (500 mL) was added TEA (37.1 mL, 368 mmol) at 0° C. After 10 minutes stirring, benzaldehyde (18.7 g, 176 mmol) was added to the reaction. The mixture was stirred at 0° C. for 10 minutes, then NaBH₄ (8.4 g, 221 mmol) was added. The mixture was stirred at 0° C. to room temperature overnight. The reaction was quenched with 200 mL of sat.NH₄Cl. The mixture was extracted with EtOAc (500 mL×2). The organic layer was concentrated. The crude product was purified by chromatography using Petroleum ether/EtOAc=10:1 to 2:1 to give the title compound (10 g, 31%) as a yellow oil.

¹HNMR (300 MHz, CDCl₃): δ 7.39-7.31 (m, 5H), 4.70 (s, 2H), 3.87-3.66 (m, 5H), 1.19 (d, J=4.5 Hz, 3H).

### Description B-15

### methyl 3-(N-benzyl-2-chloroacetamido)-2-hydroxybutanoate (D B-15)

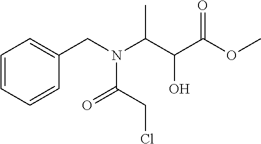 [SMILES: COC(=O)C(O)C(C)N(Cc1ccccc1)C(=O)CCl]

A mixture of methyl 3-(benzylamino)-2-hydroxybutanoate (10 g, 44.8 mmol) in DCM (200 mL) was added DIPEA (11.6 g, 89.6 mmol) followed by 2-chloroacetyl chloride (6.07 g, 54 mmol).

The mixture was stirred at 0° C. for 1 hour. The mixture was washed with water (200 mL), extracted with DCM (200 mL). The organic layer was concentrated. The crude product was purified by chromatography using Petroleum ether/EtOAc=4:1 to 1:1 to give compound (5.1 g, 38%) as brown oil.

¹HNMR (300 MHz, CDCl₃): δ 7.45-7.28 (m, 5H), 4.75 (s, 1H), 4.41-4.12 (m, 2H), 4.17-4.12 (m, 2H), 3.76-3.62 (m, 4H), 1.29-1.18 (m, 3H).

### Description B-16

### methyl 4-benzyl-3-methyl-5-oxomorpholine-2-carboxylate (D B-16)

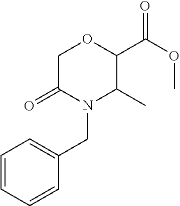 [SMILES: COC(=O)C1OCC(=O)N(Cc2ccccc2)C1C]

A mixture of methyl 3-(N-benzyl-2-chloroacetamido)-2-hydroxybutanoate (5.1 g, 17.0 mmol) in THF (60 min) at 0° C. was added NaH (1.36 g, 34 mmol, 60% in mineral oil). The mixture was stirred at 0° C. to room temperature overnight. The reaction was quenched with 20 mL of sat.NH₄Cl. The mixture was extracted with EtOAc (200 mL). The organic layer was washed with water (100 mL), and concentrated. The crude product was purified by chromatography using Petroleum ether/EtOAc=4:1 to 1:1 to give the title compound (3.4 g, 77%) as a yellow oil.

¹HNMR (300 MHz, CDCl₃): δ 7.37-7.25 (m, 5H), 5.50 (d, J=15.0 Hz, 1H), 4.34-4.19 (m, 3H), 3.82-3.71 (m, 5H), 1.25-1.19 (m, 3H).

### Description B-17

### (4-benzyl-3-methylmorpholin-2-yl)methanol (D B-17)

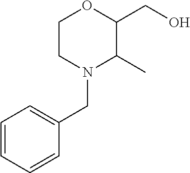 [SMILES: CC1C(CO)OCCN1Cc1ccccc1]

To a mixture of methyl 4-benzyl-3-methyl-5-oxomorpholine-2-carboxylate (3.4 g, 12.9 mmol) in THF (50 mL) was added LiAlH₄ (980 mg, 25.8 mmol) at 0° C. The mixture was stirred at 0° C. for 30 minutes, then the mixture was stirred at room temperature for 1 hour. The reaction was quenched with 10 mL of MeOH followed by sat.potassium sodium tartrate (20 mL). The mixture was diluted with 20 mL of EtOAc and stirred at room temperature for 1 hour. Na₂SO₄ was added to the mixture. The mixture was filtrated and concentrated. The crude product was purified by chromatography using Petroleum ether/EtOAc=2:1 to 1:1 to give compound (1.6 g, 82%) as yellow oil.

¹HNMR (300 MHz, CDCl₃): δ 7.38-7.29 (m, 5H), 4.20 (d, J=13.2 Hz, 1H), 4.07 (dd, J=12.3, 3.3 Hz, 1H), 3.80-3.71 (m, 2H), 3.61-3.53 (m, 2H), 3.15 (d, J=13.2 Hz, 1H), 2.82 (br s, 1H), 2.70 (dd, J=11.7, 0.3 Hz, 1H), 2.37 (dt, J=11.7, 3.3 Hz, 1H), 2.15 (dd, J=9.6, 2.7 Hz, 1H), 1.29 (d, J=6.3 Hz, 3H).

### Description B-18

### (3-methylmorpholin-2-yl)methanol hydrochloride (D B-18)

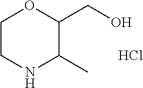

A mixture of (4-benzyl-3-methylmorpholin-2-yl)methanol (1.6 g, 7.2 mmol), Pd/C (320 mg, 20% W) in MeOH (10 mL) was added conc.HCl (3 drops). The mixture was stirred at 50° C. under H₂ (50 psi) overnight. The mixture was filtered and concentrated to give the title compound (1.0 g, 86%) as yellow oil.

¹HNMR (400 MHz, CDCl₃): δ 3.87 (dd, J=4.0, 1.6 Hz, 1H), 3.84-3.69 (m, 1H), 3.62 (dt, J=10.4, 3.6 Hz, 1H), 3.55-3.51 (m, 1H), 3.48 (s, 1H), 3.46-3.39 (m, 1H), 3.03-2.94 (m, 2H), 2.62-2.57 (m, 1H), 1.17 (d, J=6.4 Hz, 3H).

### Description B-19

### (4-(6-iodo-2-methoxypyrimidin-4-yl)-3-methylmorpholin-2-yl)methanol (D B-19)

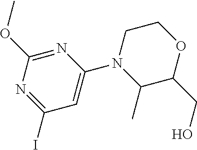 [SMILES: COc1nc(I)cc(N2CCOC(CO)C2C)n1]

To a solution of 4,6-diiodo-2-methoxypyrimidine (978 mg, 3 mmol), (3-methylmorpholin-2-yl)methanol hydrochloride (500 mg, 3 mmol) and TEA (909 mg, 9 mmol) in i-PrOH (10 mL) was stirred at 30° C. overnight. The mixture was diluted with H₂O (50 mL), extracted with EtOAc (30 mL×3). The organic layer was dried over Na₂SO₄, filtered and concentrated. The residue was purified by silica gel chromatography column (petroleum ether/EtOAc=1/1) to give the title compound (605 mg, 55%) as a colorless oil.

¹HNMR (400 MHz, CDCl₃): δ 6.68 (s, 1H), 4.18-4.08 (m, 2H), 3.99-3.90 (m, 7H), 3.71-3.67 (m, 1H), 3.35-3.30 (m, 1H), 2.05-2.02 (m, 1H), 1.36 (d, J=6.8 Hz, 3H).

### Description B-20

### tert-butyl 4-(1-(6-(2-(hydroxymethyl)-3-methylmorpholino)-2-methoxypyrimidin-4-yl)-5-methyl-1H-indazol-6-yl)piperidine-1-carboxylate (D B-20)

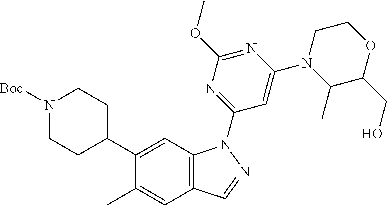 [SMILES: COc1nc(N2CCOC(CO)C2C)cc(-n2ncc3cc(C)c(C4CCN(C(=O)OC(C)(C)C)CC4)cc32)n1]

To a solution of tert-butyl 4-(5-methyl-1H-indazol-6-yl)piperidine-1-carboxylate (476 mg, 1.51 mmol) and (4-(6-iodo-2-methoxypyrimidin-4-yl)-3-methylmorpholin-2-yl)methanol (605 mg, 1.66 mmol), N,N′-dimethylcyclohexane-1,2-diamine (214 mg, 1.51 mmol), CuI (143 mg, 0.75 mmol) and K₃PO₄ (640 mg, 3.02 mmol) in toluene (3 mL) was stirred at 100° C. for 5 hours. The mixture was diluted with 50 mL of EtOAc and washed with NH₃H₂O (30 mL×3). The organic layer was dried over Na₂SO₄, filtered and concentrated. The residue was purified by silica gel chromatography column (Petroleum ether/EtOAc=1/1) to give the title compound (540 mg, 65%) as a white solid.

¹HNMR (400 MHz, CDCl₃): δ 8.70 (s, 1H), 8.07 (s, 1H), 7.51 (s, 1H), 6.89 (s, 1H), 4.40-4.35 (m, 3H), 4.29 (s, 3H), 4.10-3.93 (m, 4H), 3.42 (t, J=12.8 Hz, 1H), 2.98 (t, J=12.4 Hz, 1H), 2.85-2.80 (m, 2H), 2.47 (s, 3H), 1.88-1.85 (m, 2H), 1.75-1.65 (m, 4H), 1.50 (s, 9H), 1.38 (d, J=6.8 Hz, 3H).

### Description B-21

### (4-(2-methoxy-6-(5-methyl-6-(piperidin-4-yl)-1H-indazol-1-yl)pyrimidin-4-yl)-3-methylmorpholin-2-yl)methanol (D B-21)

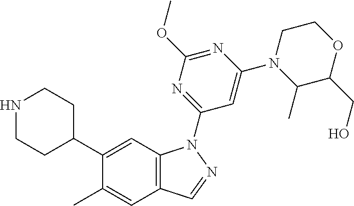 [SMILES: COc1nc(N2CCOC(CO)C2C)cc(-n2ncc3cc(C)c(C4CCNCC4)cc32)n1]

To a solution of tert-butyl 4-(1-(6-(2-(hydroxymethyl)-3-methylmorpholino)-2-methoxypyrimidin-4-yl)-5-methyl-1H-indazol-6-yl)piperidine-1-carboxylate (540 mg, 0.98 mmol) in DCM (4 mL) was added TFA (1 mL). The mixture was stirred at room for 1 hour. Sat.NaHCO₃ was added to the mixture to adjust pH >7. The mixture was diluted with H₂O (50 mL), extracted with EtOAc (30 mL×3). The organic layer was dried over Na₂SO₄, filtered and concentrated to give the title compound (442 mg, 99%) as a white solid.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 10-95-POS; flow rate: 1.5 ml/min]: Rt=1.679 min; MS Calcd.: 452, MS Found: 453 [M+H]⁺.

### Description B-22

### (4-(2-methoxy-6-(5-methyl-6-(piperidin-4-yl)-1H-indazol-1-yl)pyrimidin-4-yl)-5-methylmorpholin-2-yl)methanol (D B-22)

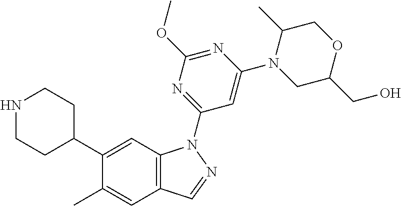 [SMILES: COc1nc(N2CC(CO)OCC2C)cc(-n2ncc3cc(C)c(C4CCNCC4)cc32)n1]

A solution of tert-butyl 4-(1-(6-(2-(hydroxymethyl)-5-methylmorpholino)-2-methoxypyrimidin-4-yl)-5-methyl-1H-indazol-6-yl)piperidine-1-carboxylate (507 mg, 0.92 mmol) in DCM (4 mL) and TFA (4 mL). The mixture was stirred at room temperature for 2 hours. The mixture was concentrated. The residue was purified by prep-TLC (DCM/MeOH=10/1) to give the title compound (400 mg, 96%) as a yellow solid.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 05-95-POS; flow rate: 1.5 ml/min]: Rt=1.756 min; MS Calcd.: 452, MS Found: 353 [M+H−100]⁺.

### Description B-23

### Mixture of tert-butyl 6-methyl-4-(((trifluoromethyl)sulfonyl)oxy)-5,6-dihydropyridine-1(2H)-carboxylate and tert-butyl 2-methyl-4-(((trifluoromethyl)sulfonyl)oxy)-5,6-dihydropyridine-1(2H)-carboxylate (D B-23)

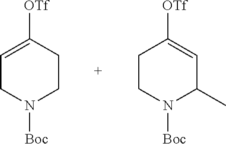 [SMILES: CC(C)(C)OC(=O)N1CC=C(OS(=O)(=O)C(F)(F)F)CC1 ; CC1C=C(OS(=O)(=O)C(F)(F)F)CCN1C(=O)OC(C)(C)C]

To a solution of tert-butyl 2-methyl-4-oxopiperidine-1-carboxylate (12.5 g, 58.7 mmol) in THF (200 mL) at −70° C. was added LiHMDS (65 mL, 64.5 mmol, 1.0 mol/L in THF). The mixture was stirred at −70° C. for 1 hour. Then N,N-Bis(trifluoromethylsulfonyl)aniline (23 g, 64.5 mmol) in THF (40 mL) was added to the reaction. The mixture was stirred at −70° C. to room temperature overnight. The reaction was quenched with 200 mL of sat. NH₄Cl (200 mL). The mixture was extracted with EtOAc (500 mL). The organic layer was washed with H₂O (200 mL), brine (100 mL) and concentrated. The crude product was purified by chromatography using Petroleum ether/EtOAc=100:1 to 10:1 to give compound (20.3 g, 100%) as yellow oil.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 10-95-POS; flow rate: 1.5 ml/min]: Rt=2.161 min; MS Calcd.: 345, MS Found: 290 [M−56+H]⁺.

### Description B-24

### Mixture of tert-butyl 6-methyl-4-(4,4,5,5-tetramethyl-1,3,2-dioxaborolan-2-yl)-5,6-dihydropyridine-1 (2H)-carboxylate and tert-butyl 2-methyl-4-(4,4,5,5-tetramethyl-1,3,2-dioxaborolan-2-yl)-5,6-dihydropyridine-1(2H)-carboxylate (D B-24)

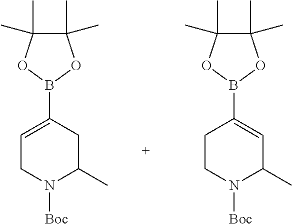

A mixture of mixture of tert-butyl 6-methyl-4-(((trifluoromethyl)sulfonyl)oxy)-5,6-dihydropyridine-1(2H)-carboxylate and tert-butyl 2-methyl-4-(((trifluoromethyl)sulfonyl)oxy)-5,6-dihydropyridine-1(2H)-carboxylate (20.3 g, 58.8 mmol), 4,4,4′,4′,5,5,5′,5′-octamethyl-2,2′-bi(1,3,2-dioxaborolane) (14.4 g, 58.8 mmol), Pd(dppf)Cl₂(4.8 g, 5.88 mol) and KOAc (11.5 g, 117.7 mmol) in 1,4-dioxane (300 mL) under N₂ was stirred at 100° C. for 4 hours. The mixture was concentrated with silica gel and purified by chromatography using Petroleum ether/EtOAc=20:1 to 10:1 to give the title compound (19 g, 100%) as yellow oil.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 40-95-POS; flow rate: 1.5 ml/min]: Rt=2.279 min; MS Calcd.: 323, MS Found: 268 [M−56+H]⁺.

### Description B-25

### Mixture of tert-butyl 2-methyl-4-(5-methyl-1H-indazol-6-yl)-5,6-dihydropyridine-1(2H)-carboxylate and tert-butyl 6-methyl-4-(5-methyl-1H-indazol-6-yl)-5,6-dihydropyridine-1(2H)-carboxylate (D B-25)

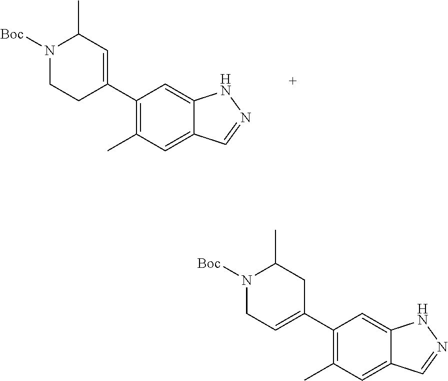 [SMILES: Cc1cc2cn[nH]c2cc1C1=CC(C)N(C(=O)OC(C)(C)C)CC1 ; Cc1cc2cn[nH]c2cc1C1=CCN(C(=O)OC(C)(C)C)C(C)C1]

A mixture of mixture of tert-butyl 6-methyl-4-(4,4,5,5-tetramethyl-1,3,2-dioxaborolan-2-yl)-5,6-dihydropyridine-1(2H)-carboxylate and tert-butyl 2-methyl-4-(4,4,5,5-tetramethyl-1,3,2-dioxaborolan-2-yl)-5,6-dihydropyridine-1(2H)-carboxylate (9.5 g, 29.2 mmol), 6-bromo-5-methyl-1H-indazole (4.1 g, 19.5 mmol), Pd(dppf)Cl₂ (1.59 g, 1.95 mmol) and K₂CO₃ (8.07 g, 58.5 mmol) in 120 mL of 1,4-dioxane/water (v/v=5/1) under N₂ was stirred at 100° C. for 4 hours. The mixture was concentrated with silica gel and purified by chromatography using petroleum ether/EtOAc=10/1 to 4/1 to give the title compound (5.0 g, 52%) as yellow oil.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 10-95-POS; flow rate: 1.5 ml/min]: Rt=2.311 min; MS Calcd.: 327, MS Found: 328 [M+H]⁺.

### Description B-26

### tert-butyl 2-methyl-4-(5-methyl-1H-indazol-6-yl)piperidine-1-carboxylate (D B-26)

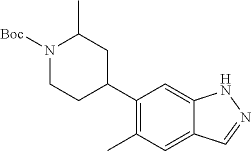 [SMILES: Cc1cc2cn[nH]c2cc1C1CCN(C(=O)OC(C)(C)C)C(C)C1]

A mixture of mixture of tert-butyl 2-methyl-4-(5-methyl-1H-indazol-6-yl)-5,6-dihydropyridine-1(2H)-carboxylate and tert-butyl 6-methyl-4-(5-methyl-1H-indazol-6-yl)-5,6-dihydropyridine-1(2H)-carboxylate (5.0 g, 15.3 mmol) and Pd/C (1.0 g, 20% W) in MeOH (100 mL) under H₂ (50 psi) was stirred at 50° C. for 7 days. The mixture was concentrated with silica gel and purified by chromatography using Petroleum ether/EtOAc=2:1 to 1:1 to give the title compound (2.65 g, 53%) as yellow oil.

¹HNMR (400 MHz, CDCl₃): δ 10.12 (br s, 1H), 7.95 (s, 1H), 7.52 (d, J=8.0 Hz, 1H), 7.30 (d, J=6.8 Hz, 1H), 4.67-4.20 (m, 1H), 4.02-3.81 (m, 1H), 3.32-3.01 (m, 2H), 2.44 (d, J=9.2 Hz, 3H), 1.75-1.66 (m, 4H), 1.51 (s, 9H), 1.27-1.26 (m, 3H)

### Description B-27

### tert-butyl 4-(1-(6-((S)-2-(hydroxymethyl)morpholino)-2-methylpyrimidin-4-yl)-5-methyl-1H-indazol-6-yl)-2-methylpiperidine-1-carboxylate

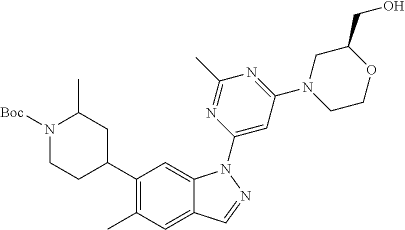 [SMILES: Cc1nc(N2CCO[C@H](CO)C2)cc(-n2ncc3cc(C)c(C4CCN(C(=O)OC(C)(C)C)C(C)C4)cc32)n1]

A mixture of tert-butyl 2-methyl-4-(5-methyl-1H-indazol-6-yl)piperidine-1-carboxylate (500 mg, 1.52 mmol), (S)-(4-(6-iodo-2-methylpyrimidin-4-yl)morpholin-2-yl)methanol (559 mg, 1.67 mmol), N,N′-dimethylcyclohexane-1,2-diamine (216 mg, 1.52 mmol), CuI (144 mg, 0.76 mmol) and K₃PO₄ (644 mg, 3.04 mmol) in toluene (5 mL) was stirred at 100° C. for 3 hours. The mixture was diluted with EtOAc (60 mL), washed with NH₃H₂O (30 mL) and brine (30 mL), dried over Na₂SO₄, filtered and concentrated. The residue was purified by silica gel chromatography column (petroleum ether/EtOAc=1:1) to give the title compound (285 mg, 35%) as yellow oil.

¹H-NMR (CDCl₃, 400 MHz): δ 8.78 (s, 1H), 8.10 (s, 1H), 7.53 (s, 1H), 6.99 (s, 1H), 4.37-4.30 (m, 2H), 4.12-4.09 (m, 2H), 3.84-3.69 (m, 5H), 3.53 (s, 1H), 3.18-3.11 (m, 1H), 3.01-2.95 (m, 3H), 2.66 (s, 3H), 2.53-2.42 (m, 3H), 1.81-1.56 (m, 4H), 1.52 (s, 9H), 1.35-1.32 (m, 3H).

### Description B-28

### (R)-(4-(6-iodo-2-methylpyrimidin-4-yl)morpholin-2-yl)methanol (D B-28)

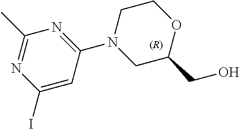

To a solution of (R)-morpholin-2-ylmethanol (300 mg, 2.56 mmol) and DIEA (992 mg, 7.68 mmol) in EtOH (10 mL) was added 4,6-diiodo-2-methylpyrimidine (885 mg, 2.56 mmol). The reaction was stirred at room temperature overnight. Solvent was removed in vacuum and the residue was purified by silica gel chromatography (eluted with PE/EtOAc=4:1) to give product (470 mg, yield 54.8%) as a pale yellow solid.

¹H NMR (400 MHz, CDCl₃): δ 6.79 (s, 1H), 4.16-4.06 (m, 2H), 4.06-4.02 (m, 1H), 3.79-3.73 (m, 1H), 3.70-3.57 (m, 3H), 3.04 (td, J=13.2, 3.6 Hz, 1H), 2.91 (dd, J=12.8, 10.4 Hz, 1H), 2.47 (s, 3H), 1.94 (t, J=6.0 Hz, 1H).

### Description B-29

### (S)-(4-(6-iodo-2-methylpyrimidin-4-yl)morpholin-2-yl)methanol (D B-29)

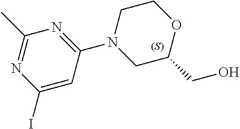

To a solution of (S)-morpholin-2-ylmethanol (300 mg, 2.56 mmol) and DIEA (992 mg, 7.68 mmol) in EtOH (10 mL) and THF (20 mL) was added 4,6-diiodo-2-methylpyrimidine (885 mg, 2.56 mmol). The reaction was stirred at room temperature overnight. Solvent was removed in vacuum and the residue was purified by silica gel chromatography (eluted with PE/EtOAc=4:1) to give product (420 mg, yield 48.8%) as a pale yellow solid.

LC-MS [mobile phase: from 90% water (0.1% FA) and 10% CH₃CN (0.1% FA) to 5% water (0.1% FA) and 95% CH₃CN (0.1% FA) in 2.0 min]: Rt=0.25 min; MS Calcd.: 335.1 MS Found: 336.0 [M+H]⁺.

### Description B-30

### ((2S)-4-(2-methyl-6-(5-methyl-6-(2-methyl piperidin-4-yl)-1H-indazol-1-yl)pyrimidin-4-yl)morpholin-2-yl)methanol (D B-30)

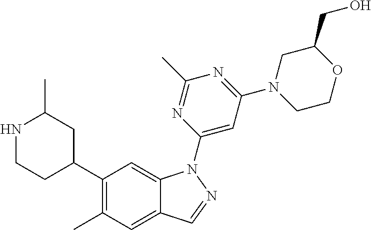 [SMILES: Cc1nc(N2CCO[C@H](CO)C2)cc(-n2ncc3cc(C)c(C4CCNC(C)C4)cc32)n1]

To a solution of tert-butyl 4-(1-(6-((S)-2-(hydroxymethyl)morpholino)-2-methylpyrimidin-4-yl)-5-methyl-1H-indazol-6-yl)-2-methylpiperidine-1-carboxylate (285 mg, 0.53 mmol) in DCM (4 mL) was added TFA (1 mL). The mixture was stirred at room temperature for 2 hours. Sat. NaHCO₃ was added to the mixture to adjust pH=9-10. The mixture was diluted with H₂O (30 mL) and extracted with DCM (30 mL×2). The organic layer was dried over Na₂SO₄, filtered and concentrated to give compound (220 mg, 95%) as yellow oil.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 10-95-POS; flow rate: 1.5 ml/min]: Rt=1.758 min; MS Calcd.: 436, MS Found: 437 [M+H]⁺.

### Description B-31

### (R)-(4-(6-iodo-2-methoxypyrimidin-4-yl)morpholin-2-yl)methanol (D B-31)

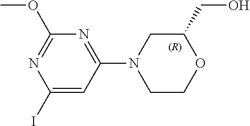

4,6-Diiodo-2-methoxypyrimidine (724 mg, 2.0 mmol) was added to the solution of (R)-morpholin-2-ylmethanol (235 mg, 2.0 mmol) and Et₃N (0.4 mL in MeOH (15 mL) at rt and the reaction was stirred at rt for 1 hour until all solid was dissolved. The reaction solution was concentrated and the residue was purified by silica gel chromatography (eluted with PE/EtOAc=2/1-1/1) to give product (680 mg, yield 97%) as a white solid.

¹H NMR (400 MHz, CDCl₃): δ 6.65 (s, 1H), 4.15˜4.01 (m, 3H), 3.91 (s, 3H), 3.77˜3.72 (m, 1H), 3.69˜3.57 (m, 3H), 3.10˜3.06 (m, 1H), 2.95˜2.88 (m, 1H), 1.96˜1.92 (m, 1H).

### Description B-32

### tert-butyl 4-(1-(6-((R)-2-(hydroxymethyl)morpholino)-2-methoxypyrimidin-4-yl)-5-methyl-1H-indazol-6-yl)-2-methylpiperidine-1-carboxylate (D B-32)

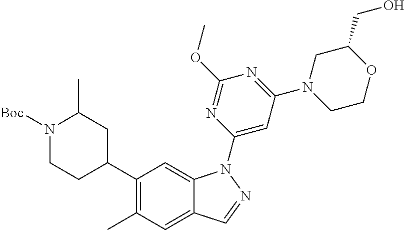 [SMILES: COc1nc(N2CCO[C@@H](CO)C2)cc(-n2ncc3cc(C)c(C4CCN(C(=O)OC(C)(C)C)C(C)C4)cc32)n1]

A mixture of tert-butyl 2-methyl-4-(5-methyl-1H-indazol-6-yl)piperidine-1-carboxylate (500 mg, 1.52 mmol), (R)-(4-(6-iodo-2-methoxypyrimidin-4-yl)morpholin-2-yl)methanol (586 mg, 1.67 mmol), N,N′-dimethylcyclohexane-1,2-diamine (216 mg, 1.52 mmol), CuI (144 mg, 0.76 mmol) and K₃PO₄ (644 mg, 3.04 mmol) in toluene (5 mL) was stirred at 100° C. for 3 hours. The mixture was diluted with EtOAc (60 mL), washed with NH₃H₂O (30 mL) and brine (30 mL), dried over Na₂SO₄, filtered and concentrated. The residue was purified by silica gel chromatography column (petroleum ether/EtOAc=1:2) to give compound (315 mg, 37%) as a yellow oil.

¹H-NMR (CDCl₃, 400 MHz): δ 8.70 (s, 1H), 8.07 (s, 1H), 7.51 (s, 1H), 6.85 (s, 1H), 4.30-4.27 (m, 3H), 4.05-4.01 (m, 7H), 3.81-3.63 (m, 7H), 3.26-2.88 (m, 5H), 2.48-2.45 (m, 3H), 1.97-1.92 (m, 2H), 1.50 (s, 9H).

### Description B-33

### ((2R)-4-(2-methoxy-6-(5-methyl-6-(2-methylpiperidin-4-yl)-1H-indazol-1-yl)pyrimidin-4-yl)morpholin-2-yl)methanol (D B-33)

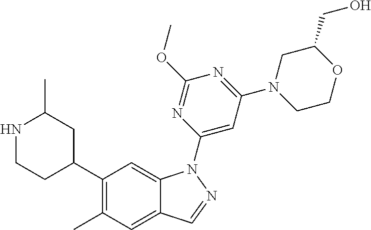 [SMILES: COc1nc(N2CCO[C@@H](CO)C2)cc(-n2ncc3cc(C)c(C4CCNC(C)C4)cc32)n1]

To a solution of tert-butyl 4-(1-(6-((R)-2-(hydroxymethyl)morpholino)-2-methoxypyrimidin-4-yl)-5-methyl-1H-indazol-6-yl)-2-methylpiperidine-1-carboxylate (315 mg, 0.57 mmol) in DCM (4 mL) was added TFA (1 mL). The mixture was stirred at room temperature for 2 hours. Sat. NaHCO₃ was added to the mixture to adjust pH=9-10. The mixture was diluted with H₂O (30 mL) and extracted with DCM (30 mL×2). The organic layer was dried over Na₂SO₄, filtered and concentrated to give compound (221 mg, 86%) as yellow oil.

¹H-NMR (CDCl₃, 400 MHz): δ 8.78 (s, 1H), 8.08 (s, 1H), 7.55 (s, 1H), 6.85 (s, 1H), 5.30 (s, 1H), 4.32-4.23 (m, 2H), 4.12 (s, 3H), 4.07-4.03 (m, 2H), 3.79-3.55 (m, 5H), 3.39-3.30 (m, 2H), 3.17-3.11 (m, 2H), 2.99-2.93 (m, 1H), 2.47 (s, 3H), 2.12-2.04 (m, 2H), 1.94-1.91 (m, 1H), 1.60-1.42 (m, 4H).

### Description B-34

### (S)-(4-(6-iodo-2-methoxypyrimidin-4-yl)morpholin-2-yl)methanol (D B-34)

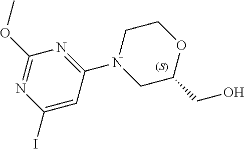

4,6-Diiodo-2-methoxypyrimidine (680 mg, 1.9 mmol) was added to the solution of (S)-morpholin-2-ylmethanol (235 mg, 2.0 mmol) and Et₃N (0.4 mL0 in MeOH (15 minL) at RT and the reaction was stirred at RT for 1 hour until all solid was dissolved. Then the reaction solution was concentrated and the residue was purified by silica gel chromatography (eluted with PE/EtOAc=2/1-2/1) to give product (680 mg, yield 97%) as a colorless oil.

¹H NMR (400 MHz, CDCl₃): δ 6.65 (s, 1H), 4.15-4.01 (m, 3H), 3.91 (s, 3H), 3.77-3.72 (m, 1H), 3.69-3.57 (m, 3H), 3.10-3.06 (m, 1H), 2.95-2.88 (m, 1H), 1.96-1.92 (m, 1H).

### Description B-35

### tert-butyl 4-(1-(6-((S)-2-(hydroxymethyl)morpholino)-2-methoxypyrimidin-4-yl)-5-methyl-1H-indazol-6-yl)-2-methylpiperidine-1-carboxylate (D B-35)

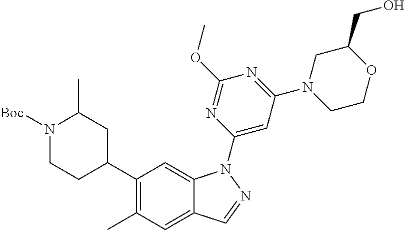 [SMILES: COc1nc(N2CCO[C@H](CO)C2)cc(-n2ncc3cc(C)c(C4CCN(C(=O)OC(C)(C)C)C(C)C4)cc32)n1]

A mixture of tert-butyl 2-methyl-4-(5-methyl-1H-indazol-6-yl)piperidine-1-carboxylate (500 mg, 1.52 mmol), (S)-(4-(6-iodo-2-methoxypyrimidin-4-yl)morpholin-2-yl)methanol (587 mg, 1.67 mmol), N,N′-dimethylcyclohexane-1,2-diamine (216 mg, 1.52 mmol), CuI (144 mg, 0.76 mmol) and K₃PO₄ (644 mg, 3.04 mmol) in toluene (3 mL) was stirred at 100° C. for 5 hours. The mixture was diluted with EtOAc (50 mL), washed with NH₃H₂O (30 mL×3), dried over Na₂SO₄, filtered and concentrated. The residue was purified by silica gel chromatography column (petroleum ether/EtOAc=1:1) to give compound (400 mg, 48%) as a white solid.

¹H-NMR (CDCl₃, 400 MHz): δ 8.72 (d, J=16 Hz, 1H), 8.07 (s, 1H), 7.51 (s, 1H), 6.85 (s, 1H), 4.30-4.25 (m, 4H), 4.21-4.03 (m, 7H), 3.92-3.64 (m, 6H), 3.28-2.94 (m, 5H), 2.48-2.45 (m, 3H), 1.98 (s, 2H), 1.50 (s, 9H).

### Description B-36

### ((2S)-4-(2-methoxy-6-(5-methyl-6-(2-methyl piperidin-4-yl)-1H-indazol-1-yl)pyrimidin-4-yl)morpholin-2-yl)methanol (D B-36)

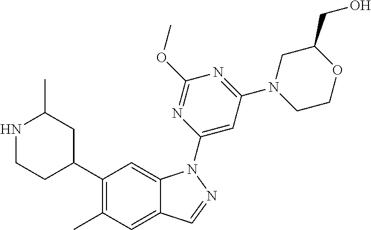 [SMILES: COc1nc(N2CCO[C@H](CO)C2)cc(-n2ncc3cc(C)c(C4CCNC(C)C4)cc32)n1]

To a solution of tert-butyl 4-(1-(6-((S)-2-(hydroxymethyl)morpholino)-2-methoxypyrimidin-4-yl)-5-methyl-1H-indazol-6-yl)-2-methylpiperidine-1-carboxylate (400 mg, 0.72 mmol) in DCM (4 mL) was added TFA (1 mL). The mixture was stirred at room temperature for 3 hours. Sat. NaHCO₃ was added to the mixture to adjust pH >7. The mixture was diluted with H₂O (50 mL) and extracted with EtOAc (30 mL×3). The organic layer was dried over Na₂SO₄, filtered and concentrated to give compound (327 mg, 100%) as a white solid.

¹H-NMR (CDCl₃, 400 MHz): δ 8.78 (s, 1H), 8.08 (s, 1H), 7.55 (s, 1H), 6.85 (s, 1H), 4.46-4.28 (m, 3H), 4.15 (s, 1H), 4.12 (s, 1H), 4.06-4.03 (m, 2H), 3.94-3.91 (m, 1H), 3.81-3.71 (m, 4H), 3.66-3.58 (m, 1H), 3.49-2.27 (m, 2H), 3.17-3.09 (m, 2H), 2.99-2.95 (m, 1H), 2.47 (s, 2H), 2.42 (s, 1H), 2.11 (s, 1H), 1.94-1.86 (m, 2H), 1.59-1.57 (m, 1H), 1.43-1.41 (m, 1H), 1.28-1.24 (m, 1H).

### Description B-37

### tert-butyl 4-(1-(6-((R)-2-(hydroxymethyl)morpholino)-2-methylpyrimidin-4-yl)-5-methyl-1H-indazol-6-yl)-2-methylpiperidine-1-carboxylate (D B-37)

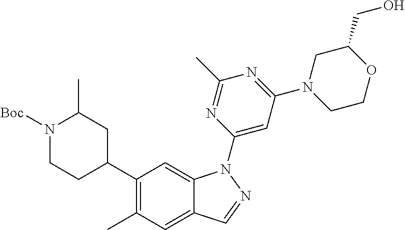 [SMILES: Cc1nc(N2CCO[C@@H](CO)C2)cc(-n2ncc3cc(C)c(C4CCN(C(=O)OC(C)(C)C)C(C)C4)cc32)n1]

A mixture of tert-butyl 2-methyl-4-(5-methyl-1H-indazol-6-yl)piperidine-1-carboxylate (500 mg, 1.52 mmol), (R)-(4-(6-iodo-2-methylpyrimidin-4-yl)morpholin-2-yl)methanol (560 mg, 1.67 mmol), N,N′-dimethylcyclohexane-1,2-diamine (216 mg, 1.52 mmol), CuI (144 mg, 0.76 mmol) and K₃PO₄ (644 mg, 3.04 mmol) in toluene (3 mL) was stirred at 100° C. for 5 hrs. The mixture was diluted with EtOAc (50 mL), washed with NH₃H₂O (30 mL×3) and brine (30 mL), dried over Na₂SO₄, filtered and concentrated. The residue was purified by silica gel chromatography column (petroleum ether/EtOAc=1:1) to give compound (562 mg, 69%) as a white solid.

¹H-NMR (CDCl₃, 400 MHz): δ 8.80 (d, J=16.8 Hz, 1H), 8.10 (s, 1H), 7.53 (s, 1H), 6.99 (s, 1H), 5.34 (s, 1H), 4.67-4.29 (m, 5H), 4.19-4.05 (m, 4H), 3.92-3.69 (m, 7H), 3.19-2.96 (m, 5H), 2.66 (s, 3H), 2.53-2.42 (m, 2H), 1.65 (s, 9H).

### Description B-38

### ((2R)-4-(2-methyl-6-(5-methyl-6-(2-methylpiperidin-4-yl)-1H-indazol-1-yl)pyrimidin-4-yl)morpholin-2-yl)methanol (D B-38)

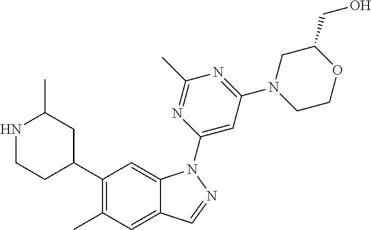 [SMILES: Cc1nc(N2CCO[C@@H](CO)C2)cc(-n2ncc3cc(C)c(C4CCNC(C)C4)cc32)n1]

To a solution of tert-butyl 4-(1-(6-((R)-2-(hydroxymethyl)morpholino)-2-methylpyrimidin-4-yl)-5-methyl-1H-indazol-6-yl)-2-methylpiperidine-1-carboxylate (562 mg, 1.05 mmol) in DCM (4 mL) was added TFA (1 mL). The mixture was stirred at room temperature for 3 hours. Sat. NaHCO₃ was added to the mixture to adjust pH >7. The mixture was diluted with H₂O (50 mL) and extracted with EtOAc (30 mL×3). The organic layer was dried over Na₂SO₄, filtered and concentrated to give compound (457 mg, 100%) as a white solid.

¹H-NMR (CDCl₃, 400 MHz): δ 8.56 (s, 1H), 8.11 (s, 1H), 7.56 (s, 1H), 6.99 (s, 1H), 4.38-4.31 (m, 4H), 4.12-4.09 (m, 2H), 3.83-3.72 (m, 7H), 3.52 (s, 1H), 3.42-3.39 (m, 2H), 3.19-2.98 (m, 6H), 2.68-2.65 (m, 3H), 2.51 (s, 2H).

### Description C-1

### 6-Bromo-5-methyl-1H-indazole (D C-1)

To a solution of 5-bromo-2,4-dimethylaniline (15.0 g, 75.0 mmol) in chloroform (150 mL) was added Ac₂O (15.0, 150 mmol) under ice bath. KOAc (8.00 g, 82.5 mmol), 18-crown-6 (10.0 g, 37.5 mmol) and isoamyl nitrite (26.3 g, 225 mmol) were added. The mixture was refluxed for 36 hrs. The reaction mixture was concentrated and the residue was dissolved in EtOAc (500 mL). The organic solution was washed with water (100 mL), dried over Na₂SO₄ and concentrated. The residue was dissolved in THF (100 mL) and NaOH (4 M, 40.0 mL, 160 mmol) was added. The mixture was stirred at rt for 1 h. The solvent was removed under vacuum and the residue was partitioned between EtOAc (400 mL) and water (200 mL). The organic layer was washed with brine, dried over Na₂SO₄ and concentrated. The crude was purified by column chromatography (PE:EtOAc from 10:1 to 5:1) to give the title compound (5.1 g, yield 32%) as an orange solid.

¹H NMR (300 MHz, CDCl₃): δ 10.20 (br s, 1H), 7.99 (s, 1H), 7.75 (s, 1H), 7.61 (s, 1H), 2.50 (s, 3H).

### Description C-2

### 6-Bromo-5-methyl-1-(tetrahydro-2H-pyran-2-yl)-1H-indazole (D C-2)

To a solution of 6-bromo-5-methyl-1H-indazole (5.10 g, 24.2 mmol) in dry DCM (120 mL) was added DHP (4.10 g, 48.4 mmol), TsOH (0.800 g, 4.80 mmol) and Mg₂SO₄ (5.0 g) at rt. The reaction mixture was heated to 35° C. and stirred for an hour. The reaction mixture was filtered and the filtrate was washed with a solution of Na₂CO₃ (10%, 100 mL), dried over Na₂SO₄ and concentrated. The crude was purified by column chromatography (PE:EtOAc from 50:1 to 20:1) to give the title compound (6.0 g, yield 84%) as an orange solid.

¹H NMR (300 MHz, CDCl₃): δ 7.90 (s, 1H), 7.84 (s, 1H), 7.55 (s, 1H), 5.63 (dd, J=9.6, 3.0 Hz, 1H), 4.05-4.00 (m, 1H), 3.78-3.70 (m, 1H), 2.58-2.44 (m, 4H), 2.20-2.02 (m, 2H), 1.78-1.65 (m, 3H).

LCMS (mobile phase: 5-95% ACN): Rt=2.19 min in 3 min; MS Calcd: 294; MS Found: 295 [M+H]⁺.

### Description C-3

### tert-Butyl 4-(5-methyl-1-(tetrahydro-2H-pyran-2-yl)-1H-indazol-6-yl)-5,6-dihydropyridine-1(2H)-carboxylate (D C-3)

To a suspension of 6-bromo-5-methyl-1-(tetrahydro-2H-pyran-2-yl)-1H-indazole (5.50 g, 18.6 mmol), tert-butyl 4-(4,4,5,5-tetramethyl-1,3,2-dioxaborolan-2-yl)-5,6-dihydropyridine-1(2H)-carboxylate (6.90 g, 22.3 mmol) and Na₂CO₃ (4.90 g, 46.5 mmol) in dioxane (150 mL) and water (130 mL) was added Pd(dppf)Cl₂ (658 mg, 0.900 mmol). The mixture was degassed with N₂ for 3 times and then stirred at 80° C. overnight. The solvent was removed under vacuum and the residue was partitioned between EtOAc (300 mL) and water (200 mL). The combined organic layers were washed with brine, dried over Na₂SO₄ and concentrated. The crude was purified by column chromatography (PE:EtOAc=10:1) to give the title compound (7.3 g, yield 99%) as a slight brown solid.

¹H NMR (400 MHz, CDCl₃): δ 7.92 (s, 1H), 7.48 (s, 1H), 7.28 (s, 1H), 5.67 (dd, J=9.6, 2.8 Hz, 1H), 5.63 (br s, 1H), 4.07-4.01 (m, 3H), 3.78-3.70 (m, 1H), 3.67-3.64 (m, 2H), 2.62-2.53 (m, 1H), 2.45-2.39 (m, 2H), 2.34 (s, 3H), 2.18-2.12 (m, 1H), 2.07-2.02 (m, 1H), 1.81-1.73 (m, 2H), 1.69-1.61 (m, 1H), 1.52 (s, 9H).

### Description C-4

### tert-Butyl 4-(5-methyl-1-(tetrahydro-2H-pyran-2-yl)-1H-indazol-6-yl)piperidine-1-carboxylate (D C-4)

To a solution of tert-butyl 4-(5-methyl-1-(tetrahydro-2H-pyran-2-yl)-1H-indazol-6-yl)-5,6-dihydropyridine-1(2H)-carboxylate (80 g, crude) in MeOH (2 L) under H₂ was added Pd/C (10 g, 12%/W). The reaction mixture was degassed for 3 times and stirred at r.t for 2 d. The mixture was filtered and the filtrate was concentrated to give the crude product as a white solid. (65.8 g)

LC-MS [mobile phase: from 30% water (0.1% FA) and 70% ACN (0.1% FA) to 5% water (0.1% FA) and 95% ACN (0.1% FA) in 2.0 min]: Rt=0.63 min; MS Calcd.: 399.2, MS Found: 400.5 [M+H]⁺.

### Description C-5

### 5-Methyl-6-(piperidin-4-yl)-1H-indazole (D C-5)

HCl/MeOH (5M, 200 mL) was added to a solution of tert-butyl 4-(5-methyl-1-(tetrahydro-2H-pyran-2-yl)-1H-indazol-6-yl)piperidine-1-carboxylate (55.4 g, 139 mmol) in MeOH (150 mL). The reaction mixture was stirred at rt overnight, then concentrated, treated with Na₂CO₃ aq. and basified with NaOH aq. to pH >12. The mixture was filtered to give the desired product as a white solid. (29.3 g, yield=98%)

LC-MS [mobile phase: from 90% water (0.1% FA) and 10% ACN (0.1% FA) to 5% water (0.1% FA) and 95% ACN (0.1% FA) in 2.0 min]: Rt=0.85 min; MS Calcd.: 215, MS Found: 216 [M+H]⁺.

### Description C-6

### 5-Methyl-6-(1-(oxetan-3-yl)piperidin-4-yl)-1H-indazole (D C-6)

NaBH₃CN (9.40 g, 149 mmol) was added to a solution of 5-methyl-6-(piperidin-4-yl)-1H-indazole (16.0 g, 74.3 mmol), oxetan-3-one (13.4 g, 223 mmol), zeolite (13.4 g) and AcOH (1.56 g, 1.63 mmol) in CH₂Cl₂/MeOH (320 mL/80 mL) at rt. The reaction mixture was stirred at rt overnight, filtered and the filtered cake was washed with CH₂Cl₂. The filtrate was washed with NaHCO₃ aq and brine. The organic part was dried over anhydrous Na₂SO₄, filtered and concentrated. The residue was purified by column (PE:EtOAc=1:1 to CH₂Cl₂:MeOH=50:1) to give the desired product as a white solid (11.9 g, yield=59%)

LC-MS [mobile phase: from 90% water (0.1% FA) and 10% ACN (0.1% FA) to 5% water (0.1% FA) and 95% ACN (0.1% FA) in 2.0 min]: Rt=0.87 min; MS Calcd.: 271, MS Found: 272 [M+H]⁺.

### Description C-7

### 4,6-Diiodo-2-methylpyrimidine (D C-7)

To a solution of NaI (11.9 g, 79.7 mmol) in HI (55%, 50 mL) was added 4,6-dichloro-2-methylpyrimidine (10.0 g, 61.3 mmol) in portions. The resulting suspension was heated to 40° C. and stirred for 1 hour. The reaction mixture was cooled and filtered. The solid was washed with water and then triturated with methanol (50 mL). The mixture was filtered to give the title compound (9.0 g, yield 42%) as white solid.

¹H NMR (400 MHz, CDCl₃): δ 8.07 (s, 1H), 2.67 (s, 3H).

LCMS (mobile phase: 5-95% acetonitrile in 2.5 min): Rt=1.59 min, MS Calcd: 346; MS Found: 347 [M+H]⁺.

### Description C-8

### (S)-4-(6-Iodo-2-methoxypyrimidin-4-yl)-3-methylmorpholine (D C-8)

To a solution of 4,6-diiodo-2-methoxypyrimidine (1.49 g, 4.12 mmol), (S)-3-methylmorpholine hydrochloride (500 mg, 3.63 mmol) and TEA (1.25 g, 12.36 mmol) in i-PrOH (10 mL) and DMSO (10 mL) was stirred at room temperature for 18 hours. The mixture was diluted with H₂O (20 mL) and extracted with EtOAc (20 mL×3). The organic phase was dried over Na₂SO₄, filtered and concentrated. The residue was purified by silica gel chromatography column (petroleum ether/EtOAc=5/1) to give the title compound (1.16 g, 95%) as yellow oil.

LC-MS [column: C₁₈; column size: 4.6×50 mm; mobile phase: B (ACN) A (0.02% NH₄OAc); gradient (B %)]: Rt=2.173 min, MS Calcd.: 335, MS Found: 336 [M+H]⁺.

### Description C-9

### (S)-tert-Butyl 4-(1-(2-methoxy-6-(3-methylmorpholino)pyrimidin-4-yl)-5-methyl-1H-indazol-6-yl)piperidine-1-carboxylate (D C-9)

A mixture of 5-methyl-6-(1-(oxetan-3-yl)piperidin-4-yl)-1H-indazole (200 mg, 0.64 mmol), (S)-4-(6-iodo-2-methoxypyrimidin-4-yl)-3-methylmorpholine (319 mg, 0.95 mmol), N,N′-dimethylcyclohexane-1,2-diamine (180 mg, 1.27 mmol), CuI (60 mg, 0.32 mmol) and K₃PO₄ (269 mg, 1.27 mmol) in toluene (2 mL) was stirred at 100° C. for 2 hours. The mixture was diluted with 5 mL of H₂O and extracted with EtOAc (5 mL×2). The organic layer was dried over Na₂SO₄, filtered and concentrated. The residue was purified by pre-TLC (petroleum ether/EtOAc=1/3) to give the title compound (140 mg, 42%) as a yellow solid.

LC-MS [column: C₁₈; column size: 4.6×50 mm; mobile phase: B (ACN) A (0.02% NH₄OAc); gradient (B %)]: Rt=3.056 min, MS Calcd.: 522, MS Found: 523 [M+H]⁺.

### Description C-10

### (S)-4-(2-Methoxy-6-(5-methyl-6-(piperidin-4-yl)-1H-indazol-1-yl)pyrimidin-4-yl)-3-methylmorpholine (D C-10)

To a mixture of (S)-tert-butyl 4-(1-(2-methoxy-6-(3-methylmorpholino)pyrimidin-4-yl)-5-methyl-1H-indazol-6-yl)piperidine-1-carboxylate (140 mg, 0.268 mmol) in DCM (2 mL) was added TFA (2 mL). The mixture was stirred at room temperature for 2 hours. The reaction was diluted with sat. NaHCO₃ to adjust pH=8-9 and extracted with DCM (20 mL). The organic layer was washed with brine, dried over Na₂SO₄, filtered and concentrated. The residue was purified by silica gel chromatography column (DCM/MeOH=20/1) to give the title compound (115 mg, 100%) as yellow solid.

LC-MS [column: C₁₈; column size: 4.6×50 mm; mobile phase: B (ACN) A (0.02% NH₄OAc); gradient (B %)]: Rt=2.145 min, MS Calcd.: 422, MS Found: 423 [M+H]⁺.

### Description C-11

### 4,6-diiodo-2-methoxypyrimidine (D C-11)

To a solution of NaI (5.5 g, 36.3 mmol) in HI (55% in water, 30 mL) was added 4,6-dichloro-2-methoxypyrimidine (5 g, 27.9 mmol). The mixture was heated to 40° C. and stirred for 14 h. The reaction mixture was cooled to room temperature and poured into ice water (50 mL). The filtered was washed with ice water three times to give product as a white solid (3.2 g, yield 32%).

LC-MS [mobile phase: from 80% water (0.1% TFA) and 20% ACN (0.1% TFA) to 20% water (0.1% TFA) and 80% ACN (0.1% TFA) in 10 min, purity 100%]: Rt=4.72 min; MS Calcd.: 362, MS Found: 363 [M+H]⁺.

### Description C-12

### (S)-(4-(6-Iodo-2-methoxypyrimidin-4-yl)morpholin-3-yl)methanol (D C-12)

To a solution of 4,6-diiodo-2-methoxypyrimidine (1.51 g, 4.17 mmol), (S)-morpholin-3-ylmethanol hydrochloride (584 mg, 3.79 mmol) in i-PrOH and DMF (20 mL, V/V=1/1) was added TEA (1.15 g, 11.37 mmol). The mixture was stirred at 35° C. for overnight. The mixture was diluted with EtOAc (100 mL), washed with brine (30 mL×3). The organic layer was dried over Na₂SO₄, filtered and concentrated. The residue was purified by silica gel chromatography column (petroleum ether/EtOAc=6:1) to give the title compound (580 mg, 44%) as a colorless oil.

¹H NMR (CDCl₃, 300 MHz): δ 6.68 (s, 1H), 4.28 (br s, 1H), 4.10 (d, J=16.4 Hz, 2H), 3.92-3.80 (m, 6H), 3.64-3.16 (m, 2H), 3.30-3.28 (m, 1H), 2.57 (br s, 1H).

### Description C-13

### (R)-(4-(6-Iodo-2-methoxypyrimidin-4-yl)morpholin-3-yl)methanol (D C-13)

To a solution of 4,6-diiodo-2-methoxypyrimidine (1.18 g, 3.27 mmol), (R)-morpholin-3-ylmethanol hydrochloride (500 mg, 3.27 mmol), TEA (991 mg, 9.81 mmol) in i-PrOH (10 mL) and DMSO (4 mL) was stirred at room temperature overnight. The mixture was diluted with H₂O (50 mL), extracted with EtOAc (50 mL×3). The organic layer was washed with brine (50 mL×2), dried over Na₂SO₄, filtered and concentrated. The residue was purified by silica gel chromatography column (petroleum ether/EtOAc=1:1) to give the title compound (500 mg, 42%) as white solid.

¹H NMR (CDCl₃, 300 MHz): δ6.68 (s, 1H), 4.14-4.06 (m, 2H), 4.02-3.89 (m, 7H), 3.67-3.53 (m, 2H), 3.34-3.26 (m, 1H).

### Description C-14

### 5-Methyl-6-(piperidin-4-yl)-1H-indazole hydrochloride (D C-14)

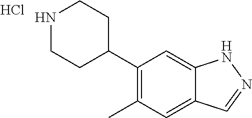 [SMILES: Cl ; Cc1cc2cn[nH]c2cc1C1CCNCC1]

tert-Butyl 4-(5-methyl-1-(tetrahydro-2H-pyran-2-yl)-1H-indazol-6-yl)piperidine-1-carboxylate (1.0 g, 2.5 mmol) was dissolved in HCl/MeOH (5 mol/L, 10 mL). Then, the mixture was stirred for 6 hrs. The mixture was concentrated under reduced pressure to afford the title compound (820 mg, yield >100%) as a light yellow solid used for next step without purification.

LC-MS: 5-95% ACN, Rt=1.13 min, MS Calcd.: 215, MS Found: 216 [M+H]⁺.

### Description C-15

### tert-Butyl 4-(5-methyl-1H-indazol-6-yl)piperidine-1-carboxylate (D C-15)

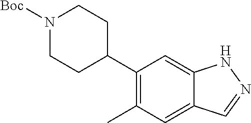 [SMILES: Cc1cc2cn[nH]c2cc1C1CCN(C(=O)OC(C)(C)C)CC1]

To a solution of 5-methyl-6-(piperdin-4-yl)-1H-indazole hydrochloride (600 mg, 2.39 mmol) in CH₃OH (10 mL) and H₂O (2 mL) was added KOH (268 mg, 4.78 mmol) and (Boc)₂O (781 mg, 3.58 mmol) under ice bath. The reaction mixture was stirred at rt for 2 hrs. The reaction mixture was diluted with water (50 mL) and extracted with EtOAc (50 mL×3). The combined organic layers were dried over Na₂SO₄ and concentrated. The residue was purified by column chromatograph (PE:EtOAc from 10:1 to 4:1) to give the title compound (353 mg, yield 47%) as a yellow oil.

¹H NMR (300 MHz, CDCl₃): δ 10.15 (br s, 1H), 7.95 (s, 1H), 7.53 (s, 1H), 7.29 (s, 1H), 4.34 (br s, 2H), 2.95-2.81 (m, 3H), 2.45 (s, 3H), 1.86-1.81 (m, 2H), 1.69-1.61 (m, 2H), 1.51 (s, 9H).

### Description C-16

### (R)-tert-Butyl 4-(1-(6-(3-(hydroxymethyl)morpholino)-2-methoxypyrimidin-4-yl)-5-methyl-1H-indazol-6-yl)piperidine-1-carboxylate (D C-16)

The title compound was prepared by a procedure similar to those described for Description 19 starting from N,N′-dimethylcyclohexane-1,2-diamine, (R)-(4-(6-iodo-2-methoxypyrimidin-4-yl)morpholin-3-yl)methanol, tert-butyl 4-(5-methyl-1H-indazol-6-yl)piperidine-1-carboxylate, CuI and K₃PO₄.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 min-05-95-POS; flow rate: 1.5 ml/min]: Rt=2.675 min; MS Calcd.: 538, MS Found: 539 [M+H]⁺.

### Description C-17

### (R)-(4-(2-Methoxy-6-(5-methyl-6-(piperidin-4-yl)-1H-indazol-1-yl)pyrimidin-4-yl)morpholin-3-yl)methanol (D C-17)

To a mixture of (R)-tert-butyl 4-(1-(6-(3-(hydroxymethyl)morpholino)-2-methoxypyrimidin-4-yl)-5-methyl-1H-indazol-6-yl)piperidine-1-carboxylate (100 mg, 0.19 mmol) in DCM (2 mL) was added TFA (2 mL). The mixture was stirred at room temperature for 3 hours. The reaction was basified with sat. NaHCO₃ (15 mL) to adjust pH=9, extracted with DCM (20 mL×3), dried over Na₂SO₄ and concentrated. The residue was purified by pre-TLC (DCM/MeOH=10/1) to give the title compound (40 mg, 48%) as a white solid.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins-05-95-POS; flow rate: 1.5 ml/min]: Rt=1.834 min; MS Calcd.: 438, MS Found: 439 [M+H]⁺.

### Description C-18

### (4-(6-Iodo-2-methoxypyrimidin-4-yl)-2-methylmorpholin-3-yl)methanol (D C-18)

To a solution of 4,6-diiodo-2-methoxypyrimidine (1.56 g, 4.78 mmol), (2-methylmorpholin-3-yl)methanol (1.6 g, 9.55 mmol) and TEA (2.89 g, 28.6 mmol) in DMSO (20 mL) was stirred at 60° C. overnight. The mixture was diluted with H₂O (30 mL), extracted with EtOAc (50 mL×3). The organic layer was dried over Na₂SO₄, filtered and concentrated. The residue was purified by silica gel chromatography column (petroleum ether/EtOAc=2/1) to give the title compound (640 mg, 37%) as yellow oil.

¹HNMR (400 MHz, CDCl₃): δ 6.61 (s, 1H), 4.07-4.03 (m, 1H), 3.91 (m, 3H), 3.73-3.56 (m, 4H), 3.31-3.30 (m, 1H), 1.93-1.90 (m, 1H), 1.58-1.56 (m, 1H), 1.12 (d, J=6.0 Hz, 3H).

### Description C-19

### tert-Butyl 4-(1-(6-(3-(hydroxymethyl)-2-methylmorpholino)-2-methoxypyrimidin-4-yl)-5-methyl-1H-indazol-6-yl)piperidine-1-carboxylate (D C-19)

To a solution of tert-butyl 4-(5-methyl-1H-indazol-6-yl)piperidine-1-carboxylate (550 mg, 1.75 mmol) and (4-(6-iodo-2-methoxypyrimidin-4-yl)-2-methylmorpholin-3-yl)methanol (640 mg, 1.75 mmol), N,N′-dimethylcyclohexane-1,2-diamine (249 mg, 1.75 mmol), CuI (166 mg, 0.88 mmol) and K₃PO₄ (742 mg, 3.50 mmol) in toluene (10 mL) was stirred at 100° C. for 4 hours. The mixture was diluted with 30 mL of H₂O and 10 mL NH₃H₂O and extracted with EtOAc (50 mL×3). The organic layer was dried over Na₂SO₄, filtered and concentrated to give the title compound (1.0 g, 100%) as a yellow solid.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 5-95-POS; flow rate: 1.5 ml/min]: Rt=2.740 min; MS Calcd.: 552, MS Found: 553 [M+H]⁺.

### Description C-20

### (4-(2-Methoxy-6-(5-methyl-6-(piperidin-4-yl)-1H-indazol-1-yl)pyrimidin-4-yl)-2-methylmorpholin-3-yl)methanol (D C-20)

A solution of tert-butyl 4-(1-(6-(3-(hydroxymethyl)-2-methylmorpholino)-2-methoxypyrimidin-4-yl)-5-methyl-1H-indazol-6-yl)piperidine-1-carboxylate (1.0 g, 1.85 mmol) in DCM (10 mL) and TFA (10 mL) was stirred at room for 30 minutes. The mixture was diluted with sat.NaHCO₃ to adjust pH=7-8. The mixture was extracted with DCM (40 mL×3). The organic layer was dried over Na₂SO₄, filtered and concentrated to give the title compound (920 mg, 100%) as a white solid.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 5-95-POS; flow rate: 1.5 ml/min]: Rt=1.850 min; MS Calcd.: 452, MS Found: 453 [M+H]⁺.

### Example A-1

### (R)-(4-(2-Methyl-6-(5-methyl-6-(1-(3-methyloxetan-3-yl)piperidin-4-yl)-1H-indazol-1-yl)pyrimidin-4-yl)morpholin-2-yl)methanol

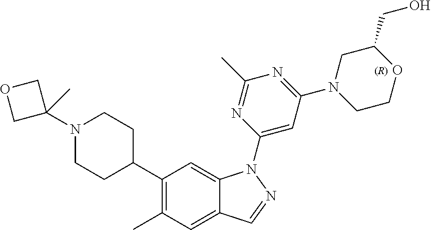

To a mixture of 5-methyl-6-(1-(3-methyloxetan-3-yl)piperidin-4-yl)-1H-indazole (65 mg, 0.228 mmol), (R)-(4-(6-iodo-2-methylpyrimidin-4-yl)morpholin-2-yl)methanol (77 mg, 0.230 mmol), CuI (44 mg, 0.23 mmol) and K₃PO₄ (98 mg, 0.46 mmol) in dry toluene (2 ml) was added N,N′-dimethylethylenediamine (41 mg, 0.46 mmol). The suspension was degassed with Ar and stirred at 100° C. for 3 hours. TLC showed the reaction was completed. The cooled reaction mixture was filtered and the filter cake was washed with CH₂Cl₂. The combined filtrate was concentrated and the residue was purified by column chromatography (eluent: CH₂Cl₂:MeOH=15:1) to give the desired product as yellow solid (62 mg, yield: 55%).

LC-MS [mobile phase: from 90% water (0.1% FA) and 10% CH₃CN (0.1% FA) to 5% water (0.1% FA) and 95% CH₃CN (0.1% FA) in 10.0 min]: Rt=5.49 min; MS Calcd: 492.28, MS Found: 493.6 [M+H]⁺.

¹H NMR (400 MHz, CDCl₃) δ 8.80 (s, 1H), 8.06 (s, 1H), 7.50 (s, 1H), 6.95 (s, 1H), 4.69 (d, J=4.8 Hz, 2H), 4.32-4.27 (m, 4H), 4.07 (d, J=9.5 Hz, 1H), 3.78-3.68 (m, 4H), 3.18-2.70 (m, 6H), 2.66 (s, 3H), 2.45 (s, 3H), 2.32 (br s, 2H), 1.93 (br s, 4H), 1.44 (s, 3H).

### Example A-2

### (S)-(4-(2-Methyl-6-(5-methyl-6-(1-(3-methyloxetan-3-yl)piperidin-4-yl)-1H-indazol-1-yl)pyrimidin-4-yl)morpholin-2-yl)methanol

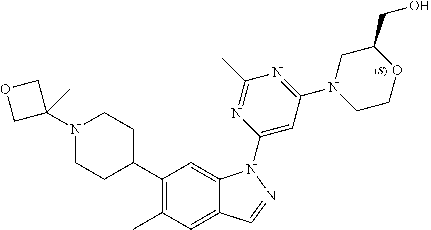

To a mixture of 5-methyl-6-(1-(3-methyloxetan-3-yl)piperidin-4-yl)-1H-indazol (65 mg, 0.228 mmol), (S)-(4-(6-iodo-2-methylpyrimidin-4-yl)morpholin-2-yl)methanol (77 mg, 0.230 mmol), CuI (44 mg, 0.23 mmol) and K₃PO₄ (98 mg, 0.46 mmol) in dry toluene (2 ml) was added N,N′-dimethylethylenediamine (41 mg, 0.46 mmol). The suspension was degassed with Ar and stirred at 100° C. for 3 hours. The cooled reaction mixture was filtered and the filtered cake was washed with CH₂Cl₂. The combined filtrate was concentrated and purified by column chromatography (eluent: CH₂Cl₂:MeOH=15:1) to afford the desired product as a yellow solid (65 mg, yield: 57%).

LC-MS [mobile phase: from 90% water (0.1% FA) and 10% CH₃CN (0.1% FA) to 5% water (0.1% FA) and 95% CH₃CN (0.1% FA) in 10.0 min]: Rt=5.49 min; MS Calcd: 492.28, MS Found: 493.6 [M+H]⁺.

¹H NMR (400 MHz, CDCl₃) δ 8.80 (s, 1H), 8.06 (s, 1H), 7.50 (s, 1H), 6.95 (s, 1H), 4.68 (br s, 2H), 4.32-4.27 (m, 4H), 4.07 (d, J=11.2 Hz, 1H), 3.78-3.68 (m, 4H), 3.18-2.72 (m, 6H), 2.66 (s, 3H), 2.45 (s, 3H), 2.32 (br s, 2H), 1.93 (br s, 4H), 1.44 (s, 3H).

### Example A-3

### (S)-(4-(2-Methoxy-6-(5-methyl-6-(1-(3-methyloxetan-3-yl)piperidin-4-yl)-1H-indazol-1-yl)pyrimidin-4-yl)morpholin-2-yl)methanol

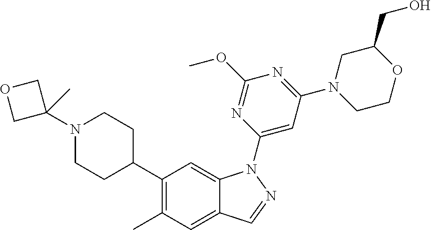 [SMILES: COc1nc(N2CCO[C@H](CO)C2)cc(-n2ncc3cc(C)c(C4CCN(C5(C)COC5)CC4)cc32)n1]

The title compound was prepared by a procedure similar to those described for E1 starting form a mixture of 5-methyl-6-(1-(3-methyloxetan-3-yl)piperidin-4-yl)-1H-indazole, (S)-(4-(6-iodo-2-methoxypyrimidin-4-yl)morpholin-2-yl)methanol, N,N′-dimethylcyclohexane-1,2-diamine, CuI and K₃PO₄ in toluene at 100° C.

¹H NMR (400 MHz, CDCl₃): δ 8.78 (s, 1H), 8.06 (s, 1H), 7.50 (s, 1H), 6.85 (s, 1H), 4.63 (d, J=5.2 Hz, 2H), 4.26-4.24 (m, 4H), 4.17 (s, 3H), 4.06 (d, J=11.6 Hz, 1H), 3.77-3.66 (m, 4H), 3.14 (t, J=14.0 Hz, 1H), 2.97 (t, J=12.0 Hz, 1H), 2.84-2.78 (m, 1H), 2.67 (d, J=10.0 Hz, 2H), 2.45 (s, 3H), 2.29 (t, J=10.0 Hz, 2H), 2.09 (br s, 1H), 1.93-1.82 (m, 4H), 1.69 (s, 3H).

LC-MS [column: C₁₈; column size: 4.6×50 mm; mobile phase: B (ACN): A (0.02% NH₄OAc); gradient (B %) in 6 min]: Rt=4.565 min; MS Calcd.: 508, MS Found: 509 [M+H]⁺.

### Example A-4

### (R)-(4-(2-Methoxy-6-(5-methyl-6-(l-(3-methyloxetan-3-yl)piperidin-4-yl)-1H-indazol-1-yl)pyrimidin-4-yl)morpholin-2-yl)methanol

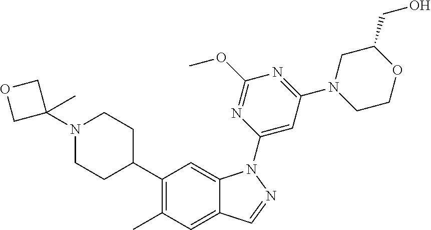 [SMILES: COc1nc(N2CCO[C@@H](CO)C2)cc(-n2ncc3cc(C)c(C4CCN(C5(C)COC5)CC4)cc32)n1]

The title compound was prepared by a procedure similar to those described for E A-1 starting form a mixture of 5-methyl-6-(1-(3-methyloxetan-3-yl)piperidin-4-yl)-1H-indazole, (R)-(4-(6-iodo-2-methoxypyrimidin-4-yl)morpholin-2-yl)methanol, N, N′-dimethylcyclohexane-1,2-diamine, CuI and K₃PO₄ in toluene at 100° C.

¹H NMR (400 MHz, CDCl₃): 58.78 (s, 1H), 8.06 (s, 1H), 7.50 (s, 1H), 6.85 (s, 1H), 4.63 (d, J=5.6 Hz, 2H), 4.31-4.24 (m, 4H), 4.17 (s, 3H), 4.06 (d, J=11.6 Hz, 1H), 3.78-3.66 (m, 4H), 3.14 (t, J=10.0 Hz, 1H), 2.97 (t, J=12.8 Hz, 1H), 2.84-2.78 (m, 1H), 2.67 (d, J=11.2 Hz, 2H), 2.45 (s, 3H), 2.29 (t, J=10.0 Hz, 2H), 2.07-2.04 (m, 1H), 1.93-1.81 (m, 4H), 1.67 (s, 3H).

LC-MS [column: C₁₈; column size: 4.6×50 mm; mobile phase: B (ACN): A (0.02% NH₄OAc); gradient (B %) in 6 min]: Rt=4.030 min; MS Calcd.: 508, MS Found: 509 [M+H]⁺.

### Examples B-1 and B-2

### (6-Methyl-4-(2-methyl-6-(5-methyl-6-(1-(oxetan-3-yl)piperidin-4-yl)-1H-indazol-1-yl)pyrimidin-4-yl)morpholin-2-yl)methanol (E B-1, Single unknown isomer 1; E B-2, single unknown isomer 2)

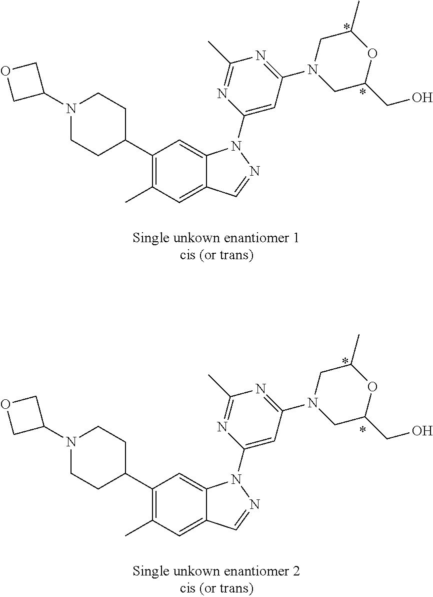 [SMILES: Cc1nc(N2CC(C)OC(CO)C2)cc(-n2ncc3cc(C)c(C4CCN(C5COC5)CC4)cc32)n1 ; Cc1nc(N2CC(C)OC(CO)C2)cc(-n2ncc3cc(C)c(C4CCN(C5COC5)CC4)cc32)n1]

To a mixture of (4-(6-iodo-2-methylpyrimidin-4-yl)-6-methylmorpholin-2-yl)methanol, isomer 1) (207 mg, 0.6 mmol), 5-methyl-6-(1-(oxetan-3-yl)piperidin-4-yl)-1H-indazole (161 mg, 0.6 mmol), CuI (113 mg, 0.6 mmol) and K₃PO₄ (251 mg, 1.2 mmol) in toluene (8 mL) was added N,N′-dimethylethylenediamine (104 mg, 1.2 mmol) under Ar. The reaction was stirred at 100° C. for 4 h. The cooled reaction mixture was filtered and the filtrate was concentrated. The residue was purified by column chromatography (eluent: PE:EtOAc=1:1, followed by CH₂Cl₂:MeOH=50:1) afforded the desired product as a yellow solid (168 mg, yield: 57%).

LC-MS [mobile phase: from 80% water (0.1% FA) and 20% CH₃CN (0.1% FA) to 5% water (0.1% FA) and 95% CH₃CN (0.1% FA) in 2.0 min]: Rt=0.99 min; MS Calcd: 492.28, MS Found: 493.5 [M+H]⁺.

The desired product was separated by chiral prep-HPLC (Method: Column: AD-H; Column size: 0.46 cm I.D.×15 cm L; Mobile phase: CO₂:EtOH (0.1% NH₃H₂O)=60:40; Flow rate: 0.5 ml/min; Wave length: UV 254 nm; Temperature: 25° C.; Sample solution in EtOH) afforded (6-methyl-4-(2-methyl-6-(5-methyl-6-(1-(oxetan-3-yl)piperidin-4-yl)-1H-indazol-1-yl)pyrimidin-4-yl)morpholin-2-yl)methanol (Single unknown isomer 1) as a white solid (80 mg, yield: 47%) and (6-methyl-4-(2-methyl-6-(5-methyl-6-(1-(oxetan-3-yl)piperidin-4-yl)-1H-indazol-1-yl)pyrimidin-4-yl)morpholin-2-yl)methanol (single unknown isomer 2) as a yellow solid (83 mg, yield: 49%).

### Example B-1

LC-MS [mobile phase: from 90% water (0.1% FA) and 10% CH₃CN (0.1% FA) to 5% water (0.1% FA) and 95% CH₃CN (0.1% FA) in 10.0 min]: Rt=5.55 min; MS Calcd: 492, MS Found: 493 [M+H]⁺.

¹H NMR (400 MHz, CDCl₃) δ 8.80 (s, 1H), 8.06 (s, 1H), 7.50 (s, 1H), 6.94 (s, 1H), 4.71 (d, J=6.4 Hz, 4H), 4.34 (d, J=11.2 Hz, 2H), 3.80-3.69 (m, 4H), 3.59-3.53 (m, 1H), 2.97 (d, J=10.0 Hz, 2H), 2.90-2.75 (m, 2H), 2.69-2.63 (m, 1H), 2.65 (s, 3H), 2.45 (s, 3H), 2.07-1.94 (m, 7H), 1.30 (d, J=6.0 Hz, 3H).

### Example B-2

LC-MS [mobile phase: from 90% water (0.1% FA) and 10% CH₃CN (0.1% FA) to 5% water (0.1% FA) and 95% CH₃CN (0.1% FA) in 10.0 min]: Rt=5.54 min; MS Calcd: 492, MS Found: 493 [M+H]⁺.

¹H NMR (400 MHz, CDCl₃) δ 8.80 (s, 1H), 8.06 (s, 1H), 7.50 (s, 1H), 6.94 (s, 1H), 4.71 (d, J=6.8 Hz, 4H), 4.34 (d, J=12.0 Hz, 2H), 3.80-3.69 (m, 4H), 3.59-3.53 (m, 1H), 2.97 (d, J=10.4 Hz, 2H), 2.84-2.78 (m, 2H), 2.68-2.63 (m, 1H), 2.65 (s, 3H), 2.45 (s, 3H), 2.07-1.94 (m, 7H), 1.30 (d, J=6.0 Hz, 3H).

### Examples B-3 and B-4

### (6-Methyl-4-(2-methyl-6-(5-methyl-6-(1-(oxetan-3-yl)piperidin-4-yl)-1H-indazol-1-yl)pyrimidin-4-yl)morpholin-2-yl)methanol (E B-3, single unknown enantiomer 3; E B-4, single unknown enantiomer 4)

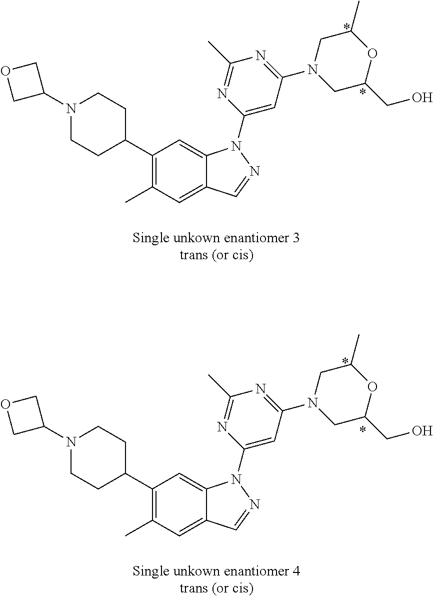 [SMILES: Cc1nc(N2CC(C)OC(CO)C2)cc(-n2ncc3cc(C)c(C4CCN(C5COC5)CC4)cc32)n1 ; Cc1nc(N2CC(C)OC(CO)C2)cc(-n2ncc3cc(C)c(C4CCN(C5COC5)CC4)cc32)n1]

The title compounds were prepared by a procedure similar to those described for example 1 and example 2 starting from N, N′-dimethylethylenediamine, (4-(6-iodo-2-methylpyrimidin-4-yl)-6-methylmorpholin-2-yl)methanol (isomer 2), 5-methyl-6-(1-(oxetan-3-yl)piperidin-4-yl)-1H-indazole, CuI and K₃PO₄.

Chiral Separation:

Method: Column: AD-H; Column size: 0.46 cm I.D.×15 cm L; Mobile phase: CO₂:EtOH (0.1% NH₃H₂O)=60:40; Flow rate: 0.5 ml/min; Wave length: UV 254 nm; Temperature: 25° C.; Sample solution in EtOH.

### Example B-3

LC-MS [mobile phase: from 90% water (0.1% FA) and 10% CH₃CN (0.1% FA) to 5% water (0.1% FA) and 95% CH₃CN (0.1% FA) in 10.0 min]: Rt=5.42 min; MS Calcd: 492, MS Found: 493 [M+H]⁺.

¹H NMR (400 MHz, CDCl₃) δ 8.80 (s, 1H), 8.06 (s, 1H), 7.50 (s, 1H), 6.93 (s, 1H), 4.71 (d, J=6.4 Hz, 4H), 4.07-4.02 (m, 2H), 3.99-3.87 (m, 2H), 3.75-3.66 (m, 3H), 3.59-3.53 (m, 1H), 3.33 (dd, J=13.2, 8.0 Hz, 1H), 2.97 (d, J=10.0 Hz, 2H), 2.86-2.80 (m, 1H), 2.64 (s, 3H), 2.45 (s, 3H), 2.07-1.93 (m, 7H), 1.26 (d, J=6.0 Hz, 3H).

### Example B-4

LC-MS [mobile phase: from 90% water (0.1% FA) and 10% CH₃CN (0.1% FA) to 5% water (0.1% FA) and 95% CH₃CN (0.1% FA) in 10.0 min]: Rt=5.41 min; MS Calcd: 492, MS Found: 493 [M+H]⁺.

¹H NMR (400 MHz, CDCl₃) δ 8.80 (s, 1H), 8.06 (s, 1H), 7.50 (s, 1H), 6.93 (s, 1H), 4.71 (d, J=6.8 Hz, 4H), 4.05 (d, J=3.6 Hz, 2H), 4.04-3.87 (m, 2H), 3.74-3.66 (m, 3H), 3.59-3.53 (m, 1H), 3.32 (dd, J=13.0, 7.5 Hz, 1H), 2.97 (d, J=10.0 Hz, 2H), 2.84 (t, J=10.8 Hz, 1H), 2.64 (s, 3H), 2.45 (s, 3H), 2.07-1.87 (m, 7H), 1.26 (d, J=6.4 Hz, 3H).

### Examples B-5 and B-6

### (5-methyl-4-(2-methyl-6-(5-methyl-6-(1-(oxetan-3-yl)piperidin-4-yl)-1H-indazol-1-yl)pyrimidin-4-yl)morpholin-2-yl)methanol (E B-5, Single unknown enantiomer 1; E B-6, Single Unknown Enantiomer 2)

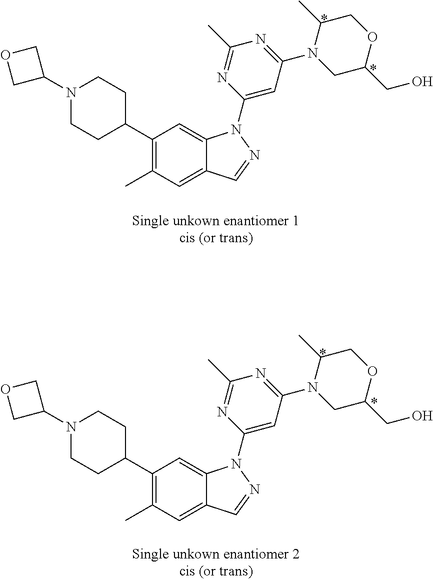 [SMILES: Cc1nc(N2CC(CO)OCC2C)cc(-n2ncc3cc(C)c(C4CCN(C5COC5)CC4)cc32)n1 ; Cc1nc(N2CC(CO)OCC2C)cc(-n2ncc3cc(C)c(C4CCN(C5COC5)CC4)cc32)n1]

The title compounds were prepared by a procedure similar to those described for E B-1 and E B-2 starting from N,N′-dimethylethylenediamine, (4-(6-iodo-2-methylpyrimidin-4-yl)-5-methylmorpholin-2-yl)methanol (isomer 1), 5-methyl-6-(1-(oxetan-3-yl)piperidin-4-yl)-1H-indazole, CuI and K₃PO₄.

Chiral Separation:

Method: Column: AD-H; Column size: 0.46 cm I.D.×15 cm L; Mobile phase: CO₂:EtOH (0.1% NH₃H₂O)=60:40; Flow rate: 0.5 mL/min; Wave length: UV 254 nm; Temperature: 25° C.; Sample solution in EtOH

### Example B-5

¹H NMR (400 MHz, CDCl3) δ 8.80 (s, 1H), 8.06 (s, 1H), 7.50 (s, 1H), 6.94 (s, 1H), 4.73-4.70 (m, 4H), 3.90-3.79 (m, 3H), 3.80-3.54 (m, 4H), 3.08-2.95 (m, 3H), 2.86-2.82 (m, 1H), 2.65 (s, 3H), 2.45 (s, 3H), 2.07-1.93 (m, 8H), 1.30 (d, J=6.8 Hz, 3H).

LC-MS [mobile phase: from 90% water (0.1% FA) and 10% CH₃CN (0.1% FA) to 5% water (0.1% FA) and 95% CH₃CN (0.1% FA) in 10.0 min]: Rt=5.55 min; MS Calcd: 492.28, MS Found: 493.5 [M+H]⁺.

Chiral HPLC: Rt: 1.892 min

### Example B-6

¹H NMR (400 MHz, CDCl3) δ 8.80 (s, 1H), 8.06 (s, 1H), 7.50 (s, 1H), 6.94 (s, 1H), 4.73-4.70 (m, 4H), 3.90-3.54 (m, 7H), 3.05-2.95 (m, 3H), 2.84-2.82 (m, 1H), 2.65 (s, 3H), 2.45 (s, 3H), 2.10-1.67 (m, 8H), 1.30 (d, J=6.8 Hz, 3H).

LC-MS [mobile phase: from 90% water (0.1% FA) and 10% CH₃CN (0.1% FA) to 5% water (0.1% FA) and 95% CH₃CN (0.1% FA) in 10.0 min]: Rt=5.54 min; MS Calcd: 492.28, MS Found: 493.6 [M+H]⁺.

Chiral HPLC: Rt: 4.966 min

### Examples B-7 and B-8

### 2-(4-(2-methyl-6-(5-methyl-6-(1-(oxetan-3-yl)piperidin-4-yl)-1H-indazol-1-yl)pyrimidin-4-yl)morpholin-2-yl)ethanol (E B-7, Single Unknown Enatiomer 1; E B-8, Single Unknown Enatiomer 2)

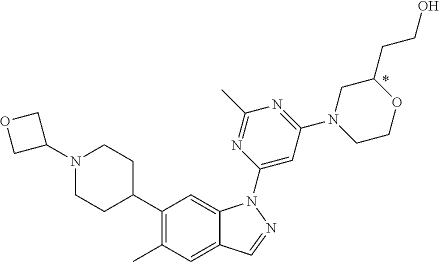 [SMILES: Cc1nc(N2CCOC(CCO)C2)cc(-n2ncc3cc(C)c(C4CCN(C5COC5)CC4)cc32)n1]

The title compounds were prepared by a procedure similar to those described for E B-1 and E B-2 starting from DMEDA, 2-(4-(6-iodo-2-methylpyrimidin-4-yl)morpholin-2-yl)ethanol, 5-methyl-6-(1-(oxetan-3-yl)piperidin-4-yl)-1H-indazole, CuI and K₃PO₄.

Chiral Separation:

Method: Column: AD-H, Column size: 0.46 cm I.D.×15 cm L, Mobile phase: CO₂:EtOH (0.05% NH₃H₂O)=60:40, Flow rate: 0.5 mL/min, Wave length: UV 205 nm, Temperature=25° C., Sample solution in EtOH

### Example B-7

LC-MS [mobile phase: from 90% water (0.1% FA) and 10% CH₃CN (0.1% FA) to 5% water (0.1% FA) and 95% CH₃CN (0.1% FA) in 10.0 min]: Rt=5.655 min; MS Calcd: 492, MS Found: 493 [M+H]⁺.

¹H NMR (400 MHz, CDCl₃) δ 8.77 (s, 1H), 8.06 (s, 1H), 7.50 (s, 1H), 6.94 (d, J=9.2 Hz, 1H), 4.71 (d, J=6.5 Hz, 4H), 4.33 (d, J=12.5 Hz, 2H), 4.04 (d, J=11.6 Hz, 1H), 3.86 (s, 2H), 3.71 (dd, J=24.6, 10.4 Hz, 2H), 3.60-3.52 (m, 1H), 3.09 (t, J=10.9 Hz, 1H), 2.97 (d, J=10.2 Hz, 2H), 2.85 (t, J=11.6 Hz, 2H), 2.65 (s, 3H), 2.45 (s, 3H), 2.09-1.99 (m, 2H), 1.94 (s, 4H), 1.85 (dd, J=12.3, 6.8 Hz, 2H).

### Example B-8

LC-MS [mobile phase: from 90% water (0.1% FA) and 10% CH₃CN (0.1% FA) to 5% water (0.1% FA) and 95% CH₃CN (0.1% FA) in 10.0 min]: Rt=5.655 min; MS Calcd: 492, MS Found: 493 [M+H]⁺.

¹H NMR (400 MHz, CDCl₃) δ 8.77 (s, 1H), 8.06 (s, 1H), 7.50 (s, 1H), 6.93 (s, 1H), 4.71 (d, J=6.5 Hz, 4H), 4.33 (d, J=11.3 Hz, 2H), 4.04 (d, J=11.3 Hz, 1H), 3.86 (s, 2H), 3.81-3.63 (m, 2H), 3.57 (dd, J=12.9, 6.4 Hz, 1H), 3.09 (t, J=11.0 Hz, 1H), 2.97 (d, J=10.0 Hz, 2H), 2.84 (t, J=11.7 Hz, 2H), 2.65 (s, 3H), 2.45 (s, 3H), 2.08-1.98 (m, 2H), 1.94 (s, 4H), 1.85 (dd, J=12.3, 6.9 Hz, 2H).

### Examples B-9-B-12

### (4-(2-methoxy-6-(5-methyl-6-(1-(oxetan-3-yl)piperidin-4-yl)-1H-indazol-1-yl)pyrimidin-4-yl)-5-methylmorpholin-2-yl)methanol (isomers 1 to 4)

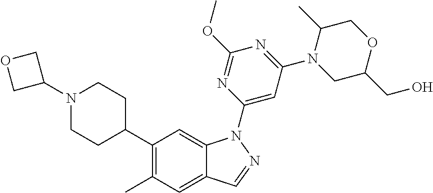 [SMILES: COc1nc(N2CC(CO)OCC2C)cc(-n2ncc3cc(C)c(C4CCN(C5COC5)CC4)cc32)n1]

To a solution of (4-(2-methoxy-6-(5-methyl-6-(piperidin-4-yl)-1H-indazol-1-yl)pyrimidin-4-yl)-5-methylmorpholin-2-yl)methanol (400 mg, 0.88 mmol), oxetan-3-one (318 mg, 4.42 mmol) and 1 drops of AcOH in DCM (20 mL) was added NaBH₃CN (110 mg, 1.76 mmol). The mixture was stirred at room temperature for 18 hours. The reaction mixture was concentrated. The residue was purified by prep-TLC (DCM/MeOH=20/1) to give the title compound (230 mg, 51%) as a yellow oil.

¹HNMR (400 MHz, CDCl₃): δ 8.76 (s, 0.5H), 8.74 (s, 0.5H), 8.07 (s, 1H), 7.51 (s, 1H), 6.81 (s, 0.5H), 6.79 (s, 0.5H), 5.30 (s, 1H), 5.28-5.33 (m, 1H), 4.90 (t, J=5.2 Hz, 2H), 4.69 (d, J=6.8 Hz, 2H), 4.58 (t, J=5.2 Hz, 2H), 4.38-4.36 (m, 0.5H), 4.22-4.19 (m, 0.5H), 4.22 (s, 1.5H), 4.19 (s, 1.5H), 4.07-4.00 (m, 1H), 3.88-3.78 (m, 2H), 3.62-3.52 (m, 2H), 2.92 (d, J=10.8 Hz, 2H), 2.83-2.81 (m, 1H), 2.39 (s, 3H), 2.26-2.25 (m, 2H), 2.01 (d, J=10.8 Hz, 2H), 1.90-1.86 (m, 2H), 1.38 (d, J=6.4 Hz, 1.5H), 1.32 (d, J=6.4 Hz, 1.5H).

Chiral separation: column: IDS; 0.46 cm×15 cm; Phase: EtOH=100; Flow rate: 0.5 ml/min;

Isomer 1 [Chiral HPLC: column: IDS; 0.46 cm×15 cm; Phase: EtOH=100; Flow rate: 0.5 ml/min; Wave length: UV 254 nm; Temperature: 35° C.]: Rt=8.382 min.

Isomer 2 [Chiral HPLC: column: IDS; 0.46 cm×15 cm; Phase: EtOH=100; Flow rate: 0.5 ml/min; Wave length: UV 254 nm; Temperature: 35° C.]: Rt=8.938 min.

Isomer 3 [Chiral HPLC: column: IDS; 0.46 cm×15 cm; Phase: EtOH=100; Flow rate: 0.5 ml/min; Wave length: UV 254 nm; Temperature: 35° C.]: Rt=9.740 min.

Isomer 4 [Chiral HPLC: column: IDS; 0.46 cm×15 cm; Phase: EtOH=100; Flow rate: 0.5 ml/min; Wave length: UV 254 nm; Temperature: 35° C.]: Rt=11.231 min.

### Examples B-13 and B-14

### (4-(2-methoxy-6-(5-methyl-6-(1-(oxetan-3-yl)piperidin-4-yl)-1H-indazol-1-yl)pyrimidin-4-yl)-3-methylmorpholin-2-yl)methanol (Isomers 1 and 2)

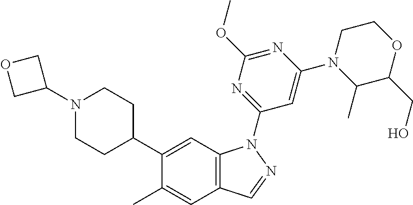 [SMILES: COc1nc(N2CCOC(CO)C2C)cc(-n2ncc3cc(C)c(C4CCN(C5COC5)CC4)cc32)n1]

To a solution of (4-(2-methoxy-6-(5-methyl-6-(piperidin-4-yl)-1H-indazol-1-yl)pyrimidin-4-yl)-3-methylmorpholin-2-yl)methanol (442 mg, 0.98 mmol), oxetan-3-one (352 mg, 4.9 mmol) and NaBH₃CN (123 mg, 1.96 mmol) in DCM (6 mL) was added catalyst AcOH. The mixture was stirred at 30° C. overnight. The reaction was quenched with 4 drops of sat.NaHCO₃ and concentrated. The residue was purified by silica gel chromatography column (DCM/MeOH=20/1) to give the title compound (180 mg, 36%) as a white solid.

¹HNMR (400 MHz, CDCl₃): δ 8.74 (s, 1H), 8.05 (s, 1H), 7.51 (s, 1H), 6.88 (s, 1H), 4.70 (d, J=6.8 Hz, 4H), 4.36 (t, J=6.8 Hz, 1H), 4.15 (s, 3H), 3.90-3.98 (m, 3H), 3.74-3.70 (m, 1H), 3.58 (t, J=6.4 Hz, 1H), 3.49 (s, 2H), 3.45-3.40 (m, 1H), 2.95 (d, J=11.2 Hz, 2H), 2.85-2.81 (m, 1H), 2.40 (s, 3H), 2.09-2.03 (m, 2H), 1.95-1.86 (m, 4H), 1.39 (d, J=6.8 Hz, 3H).

Example B-13 (isomer 1) and Example B-14 (isomer 2) were separated by chiral HPLC: column: Superchiral S-AD, 2 cm I.D.×25 cm, 5 μm; Phase: CO₂/MeOH/NH₃, H₂O=60/40/0.05; Flow rate: 30 ml/min; Wave length: 254 nm

### Isomer 1:

Chiral HPLC [column: Superchiral S-AD ID; 0.46 cm×15 cm; Phase: CO₂/EtOH/NH₃H₂O=55/45/0.05; Flow rate: 3.0 ml/min; Wave length: UV 254 nm; Temperature: 35° C.]: Rt=2.791 min.

¹H NMR (CDCl₃, 400 MHz): δ 8.75 (s, 1H), 8.07 (s, 1H), 7.51 (s, 1H), 6.88 (s, 1H), 4.72-4.67 (m, 4H), 4.36 (t, J=6.4 Hz, 1H), 4.15-4.06 (m, 5H), 4.02-3.95 (m, 3H), 3.74-3.71 (m, 1H), 3.55 (t, J=6.4 Hz, 1H), 3.46-3.39 (m, 1H), 2.93 (d, J=10.8 Hz, 2H), 2.86-2.81 (m, 1H), 2.46-2.44 (m, 4H), 2.05-1.98 (m, 2H), 1.94-1.85 (m, 4H), 1.39 (d, J=6.8 Hz, 3H).

LC-MS [column: C₁₈; column size: 4.6×50 mm; mobile phase: B (ACN) A (0.02% NH₄OAc); gradient (B %)]: Rt=3.857 min, MS Calcd.: 508, MS Found: 509 [M+H]⁺.

### Isomer 2:

Chiral HPLC [column: Superchiral S-AD ID; 0.46 cm×15 cm; Phase: CO₂/EtOH/NH₃H₂O=55/45/0.05; Flow rate: 3.0 ml/min; Wave length: UV 254 nm; Temperature: 35° C.]: Rt=6.830 min.

¹H NMR (CDCl₃, 400 MHz): δ 8.74 (s, 1H), 8.06 (s, 1H), 7.50 (s, 1H), 6.87 (s, 1H), 4.70-4.69 (m, 4H), 4.33 (t, J=6.0 Hz, 1H), 4.17-4.07 (m, 5H), 4.01-3.94 (m, 3H), 3.73-3.70 (m, 1H), 3.55 (t, J=6.4 Hz, 1H), 3.44-3.37 (m, 1H), 2.93 (d, J=10.8 Hz, 2H), 2.86-2.79 (m, 1H), 2.70 (s, 1H), 2.45 (s, 3H), 2.05-1.97 (m, 2H), 1.93-1.85 (m, 4H), 1.38 (d, J=6.8 Hz, 3H).

LC-MS [column: C₁₈; column size: 4.6×50 mm; mobile phase: B (ACN) A (0.02% NH₄OAc); gradient (B %)]: Rt=3.844 min, MS Calcd.: 508, MS Found: 509 [M+H]⁺.

### Examples B-15 and B-16

### ((2S)-4-(2-methyl-6-(5-methyl-6-(2-methyl-1-(oxetan-3-yl)piperidin-4-yl)-1H-indazol-1-yl)pyrimidin-4-yl)morpholin-2-yl)methanol (Isomers 1 and 2)

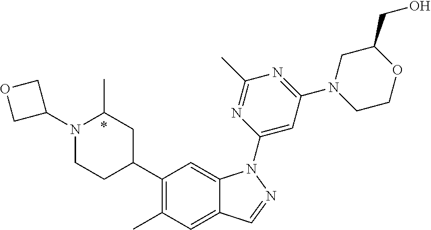 [SMILES: Cc1nc(N2CCO[C@H](CO)C2)cc(-n2ncc3cc(C)c(C4CCN(C5COC5)C(C)C4)cc32)n1]

To a solution of ((2S)-4-(2-methyl-6-(5-methyl-6-(2-methylpiperidin-4-yl)-1H-indazol-1-yl)pyrimidin-4-yl)morpholin-2-yl)methanol (220 mg, 0.50 mmol), oxetan-3-one (180 mg, 2.5 mmol) and catalyst AcOH (2 drops, 1 drop AcOH in 1 mL of DCE) in DCE (10 mL) was added NaBH₃CN (63 mg, 1.0 mmol). Another catalyst AcOH (8 drops, 1 drop AcOH in 1 mL of DCE) was added to the reaction, and the reaction was stirred at 45° C. overnight. After cooling, the reaction was quenched with sat.NaHCO₃ (4 drops) and concentrated. The residue was concentrated and purified by silica gel chromatography column (petroleum ether/EtOAc=1:4) to give compound (180 mg, 73%) as yellow oil.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 10-95-POS; flow rate: 1.5 ml/min]: Rt=2.200 min; MS Calcd.: 492, MS Found: 493 [M+H]⁺.

((2S)-4-(2-methyl-6-(5-methyl-6-(2-methyl-1-(oxetan-3-yl)piperidin-4-yl)-1H-indazol-1-yl)pyrimidin-4-yl)morpholin-2-yl)methanol (150 mg) was separated by chiral-HPLC to afford isomer 1 and isomer 2

### Chiral Prep-HPLC:

column: Superchiral S-AD, 2 cm I.D.×25 cm, 5 μm; Phase: CO₂/IPA/NH₃—H₂O=70/30/0.05; Flow rate: 30 ml/min; Wave length: 254 nm.

### Isomer 1:

LC-MS [column: C₁₈; column size: 4.6×50 mm; mobile phase: B (ACN) A (0.2% TFA); gradient (B %)]: Rt=3.543 min, MS Calcd.: 492, MS Found: 493 [M+H]⁺.

### Isomer 2:

LC-MS [column: C₁₈; column size: 4.6×50 mm; mobile phase: B (ACN) A (0.2% TFA); gradient (B %)]: Rt=3.638 min, MS Calcd.: 492, MS Found: 493 [M+H]⁺.

### Examples B-17-B-20

### ((2R)-4-(2-methoxy-6-(5-methyl-6-(2-methyl-1-(oxetan-3-yl)piperidin-4-yl)-1H-indazol-1-yl)pyrimidin-4-yl)morpholin-2-yl)methanol (Isomer 1 to 4)

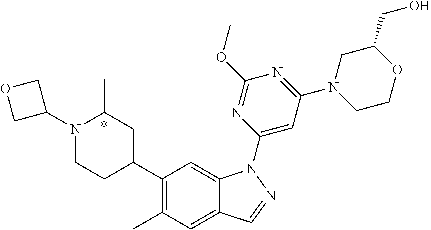 [SMILES: COc1nc(N2CCO[C@@H](CO)C2)cc(-n2ncc3cc(C)c(C4CCN(C5COC5)C(C)C4)cc32)n1]

To a solution of ((2R)-4-(2-methoxy-6-(5-methyl-6-(2-methylpiperidin-4-yl)-1H-indazol-1-yl)pyrimidin-4-yl)morpholin-2-yl)methanol (221 mg, 0.49 mmol), oxetan-3-one (176 mg, 2.45 mmol) and catalyst AcOH (2 drops, 1 drop AcOH in 1 mL of DCE) in DCE (10 mL) was added NaBH₃CN (62 mg, 0.98 mmol). The mixture was stirred at room temperature overnight. Another catalyst AcOH (7 drops, 1 drop AcOH in 1 mL of DCE) was added to the reaction. And the reaction was stirred at 45° C. overnight. After cooling, the reaction was quenched with sat.NaHCO₃ (4 drops) and concentrated. The residue was purified by silica gel chromatography column (DCM/MeOH=40:1) to give mixture compound (150 mg, 60%) as yellow oil.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 10-95-POS; flow rate: 1.5 ml/min]: Rt=2.158 min; MS Calcd.: 508, MS Found: 509 [M+H]⁺.

4 isomers were obtained from chiral separation:

### Isomer 1:

Chiral pre-HPLC: column: Chiralpak AD; 5.0 cm I.D.×25 cm L; Phase: EtOH:NH₃H₂O=100:0.1; Flow rate: 60 ml/min, Wave length: 254 nm.

¹H NMR (400 MHz, CDCl₃): δ 8.76 (s, 1H), 8.07 (s, 1H), 7.50 (s, 1H), 6.85 (s, 1H), 5.30 (s, 1H), 4.79-4.57 (m, 4H), 4.31-4.27 (m, 2H), 4.16 (s, 3H), 4.08-4.04 (m, 1H), 3.78-3.66 (m, 5H), 3.18-3.11 (m, 1H), 3.00-2.83 (m, 3H), 2.45 (s, 3H), 2.27-2.21 (m, 1H), 2.04-1.79 (m, 5H), 0.96 (d, J=6.0 Hz, 3H).

Chiral-HPLC [column: chiral pak IG, 0.46 cm I.D.×25 cm L; mobile phase: MeOH:ACN:DEA=85:15:0.1; flow rate: 1 mL/min; Wave length: 254 nm; Temperature: 35° C.]: Rt=10.518 min.

LC-MS [column: C₁₈; column size: 4.6×50 mm; mobile phase: B (ACN) A (0.02% NH₄OAc); gradient (B %)]: Rt=3.708 min, MS Calcd.: 508, MS Found: 509 [M+H]⁺.

### Isomer 2:

Chiral pre-HPLC: column: Chiralpak AD; 5.0 cm I.D.×25 cm L; Phase: EtOH:NH₃—H₂O=100:0.1; Flow rate: 60 ml/min, Wave length: 254 nm.

¹H NMR (400 MHz, CDCl₃): δ 8.76 (s, 1H), 8.07 (s, 1H), 7.51 (s, 1H), 6.85 (s, 1H), 5.30 (s, 1H), 4.73-4.65 (m, 4H), 4.31-4.25 (m, 2H), 4.15 (s, 3H), 4.08-3.97 (m, 2H), 3.78-3.68 (m, 4H), 3.24-3.11 (m, 3H), 3.00-2.94 (m, 1H), 2.74-2.64 (m, 2H), 2.46 (s, 3H), 2.03-1.89 (m, 4H), 1.06 (d, J=6.8 Hz, 3H).

Chiral-HPLC [column: chiral pak IG, 0.46 cm I.D.×25 cm L; mobile phase: MeOH:ACN:DEA=85:15:0.1; flow rate: 1 mL/min; Wave length: 254 nm; Temperature: 35° C.]: Rt=12.886 min.

LC-MS [column: C₁₈; column size: 4.6×50 mm; mobile phase: B (ACN) A (0.02% NH₄OAc); gradient (B %)]: Rt=3.709 min, MS Calcd.: 508, MS Found: 509 [M+H]⁺.

### Isomer 3:

Chiral pre-HPLC: column: Chiralpak AD; 5.0 cm I.D.×25 cm L; Phase: EtOH:NH₃—H₂O=100:0.1; Flow rate: 60 ml/min, Wave length: 254 nm.

¹H NMR (400 MHz, CDCl₃): 58.76 (s, 1H), 8.07 (s, 1H), 7.51 (s, 1H), 6.85 (s, 1H), 5.30 (s, 1H), 4.79-4.57 (m, 4H), 4.36-4.27 (m, 2H), 4.16 (s, 3H), 4.07-4.04 (m, 1H), 3.78-3.65 (m, 5H), 3.16-3.12 (m, 1H), 3.00-2.84 (m, 3H), 2.45 (s, 3H), 2.26-2.21 (m, 1H), 2.04-1.79 (m, 5H), 0.96 (d, J=6.0 Hz, 3H).

Chiral-HPLC [column: chiral pak IG, 0.46 cm I.D.×25 cm L; mobile phase: MeOH:ACN:DEA=85:15:0.1; flow rate: 1 mL/min; Wave length: 254 nm; Temperature: 35° C.]: Rt=11.795 min.

LC-MS [column: C₁₈; column size: 4.6×50 mm; mobile phase: B (ACN) A (0.02% NH₄OAc); gradient (B %)]: Rt=3.709 min, MS Calcd.: 508, MS Found: 509 [M+H]⁺.

### Isomer 4:

Chiral pre-HPLC: column: Chiralpak AD; 5.0 cm I.D.×25 cm L; Phase: EtOH:NH₃.H₂O=100:0.1; Flow rate: 60 ml/min, Wave length: 254 nm.

¹H NMR (400 MHz, CDCl₃): δ 8.76 (s, 1H), 8.07 (s, 1H), 7.51 (s, 1H), 6.85 (s, 1H), 5.30 (s, 1H), 4.73-4.65 (m, 4H), 4.31-4.25 (m, 2H), 4.15 (s, 3H), 4.08-3.97 (m, 2H), 3.78-3.66 (m, 4H), 3.23-3.15 (m, 2H), 3.00-2.94 (m, 1H), 2.74-2.64 (m, 2H), 2.46 (s, 3H), 2.04-1.90 (m, 4H), 1.77-1.73 (m, 1H), 1.06 (d, J=7.2 Hz, 3H).

Chiral-HPLC [column: chiral pak IG, 0.46 cm I.D.×25 cm L; mobile phase: MeOH:ACN:DEA=85:15:0.1; flow rate: 1 mL/min; Wave length: 254 nm; Temperature: 35° C.]: Rt=21.047 min.

LC-MS [column: C₁₈; column size: 4.6×50 mm; mobile phase: B (ACN) A (0.02% NH₄OAc); gradient (B %)]: Rt=3.712 min, MS Calcd.: 508, MS Found: 509 [M+H]⁺.

### Examples B-21-B-24

### ((2S)-4-(2-methoxy-6-(5-methyl-6-(2-methyl-1-(oxetan-3-yl)piperidin-4-yl)-1H-indazol-1-yl)pyrimidin-4-yl)morpholin-2-yl)methanol (Isomers 1 to 4)

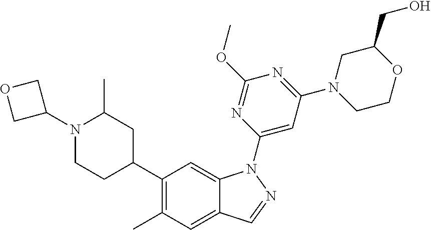 [SMILES: COc1nc(N2CCO[C@H](CO)C2)cc(-n2ncc3cc(C)c(C4CCN(C5COC5)C(C)C4)cc32)n1]

A solution of ((2S)-4-(2-methoxy-6-(5-methyl-6-(2-methylpiperidin-4-yl)-1H-indazol-1-yl)pyrimidin-4-yl)morpholin-2-yl)methanol (327 mg, 0.72 mmol), oxetan-3-one (259 mg, 3.6 mmol), NaBH₃CN (91 mg, 1.4 mmol) and catalyst AcOH in DCM (6 mL) and MeOH (1 mL) was stirred at room temperature overnight. Another catalyst AcOH (10 drops, 1 drop AcOH in 1 mL of DCM) was added to the reaction, and the reaction was stirred at 45° C. overnight. After cooling, the reaction was quenched with sat.NaHCO₃ (4 drops) and concentrated. The crude was purified by silica gel chromatography (DCM/MeOH=40:1) to give compound (300 mg, 82%) as a yellow oil.

LCMS[column: C₁₈; column size: 4.6×30 mm 5 μm; Dikwa Diamonsil plus; mobile phase: B (ACN): A (0.02% NH₄OAc+5% ACN); gradient (B %) in 4 mins. 10-95-POS; flow rate: 1.5 ml/min]: Rt=2.199 min; MS Calcd.: 508, MS Found: 509 [M+H]⁺.

4 isomers were obtained from chiral separation: Chiral HPLC: column: IGS; 0.46 cm×15 cm; Phase: Hexane/IPA=30/70; Flow rate: 1.0 ml/min; Wave length: UV 254 nm; Temperature: 35° C.

Isomer 1: chiral HPLC: Chiral HPLC: column: IGS; 0.46 cm×15 cm; Phase: MeOH/CAN=95/5; Flow rate: 1.0 ml/min; Wave length: UV 254 nm; Temperature: 35° C.; Rt=8.670 min.

Isomer 2: chiral HPLC: Chiral HPLC: column: IGS; 0.46 cm×15 cm; Phase: MeOH/CAN=95/5; Flow rate: 1.0 ml/min; Wave length: UV 254 nm; Temperature: 35° C.; Rt=12.114 min.

Isomer 3: chiral HPLC: Chiral HPLC: column: IGS; 0.46 cm×15 cm; Phase: MeOH/CAN=95/5; Flow rate: 1.0 ml/min; Wave length: UV 254 nm; Temperature: 35° C.; Rt=13.387 min.

Isomer 4: chiral HPLC: Chiral HPLC: column: IGS; 0.46 cm×15 cm; Phase: MeOH/CAN=95/5; Flow rate: 1.0 ml/min; Wave length: UV 254 nm; Temperature: 35° C.; Rt=17.380 min.

### Examples B-25-B-28

### ((2R)-4-(2-methyl-6-(5-methyl-6-(2-methyl-1-(oxetan-3-yl)piperidin-4-yl)-1H-indazol-1-yl)pyrimidin-4-yl)morpholin-2-yl)methanol

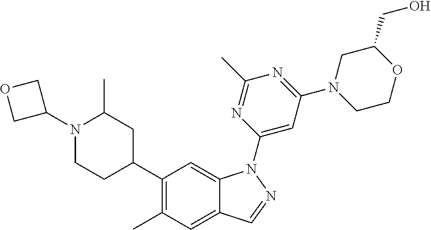 [SMILES: Cc1nc(N2CCO[C@@H](CO)C2)cc(-n2ncc3cc(C)c(C4CCN(C5COC5)C(C)C4)cc32)n1]

A solution of ((2R)-4-(2-methyl-6-(5-methyl-6-(2-methylpiperidin-4-yl)-1H-indazol-1-yl)pyrimidin-4-yl)morpholin-2-yl)methanol (457 mg, 1 mmol), oxetan-3-one (360 mg, 5 mmol), NaBH₃CN (126 mg, 2.0 mmol) and catalyst AcOH in DCM (6 mL) and MeOH (1 mL) was stirred at room temperature overnight. The reaction was quenched with NaHCO₃ (4 drops) and concentrated. The residue was concentrated and purified by flash chromatography (DCM/MeOH=40:1) to give compound (160 mg, 31%) as a white solid.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 4 mins. 10-95-POS; flow rate: 1.5 ml/min]: Rt=2.184 min; MS Calcd.: 492, MS Found: 493 [M+H]⁺.

((2R)-4-(2-methyl-6-(5-methyl-6-(2-methyl-1-(oxetan-3-yl)piperidin-4-yl)-1H-indazol-1-yl)pyrimidin-4-yl)morpholin-2-yl)methanol (160 mg) was separated by chiral-HPLC to afford isomer 1 (10 mg, 6%), isomer 2 (20 mg, 13%), isomer 3(15 mg, 9%) and isomer 4 (20 mg, 13%).

Chiral prep-HPLC: column: Superchiral S-AD, 2 cm I.D.×25 cm, 5 μm; Phase: CO₂/MeOH/NH₃H₂O=60/40/0.05; Flow rate: 30 ml/min; Wave length: 254 nm.

### Isomer 1:

Chiral HPLC [column: Superchiral S-AD ID; 0.46 cm×15 cm; Phase: CO₂/MeOH/DEA=60/40/0.05; Flow rate: 3.0 ml/min; Wave length: UV 254 nm; Temperature: 35° C.]: Rt=2.191 min.

¹H NMR (CDCl₃, 400 MHz): δ 8.79 (s, 1H), 8.06 (s, 1H), 7.50 (s, 1H), 6.95 (s, 1H), 4.83-4.77 (m, 2H), 4.69-4.60 (m, 2H), 4.33-4.30 (m, 2H), 4.09-4.05 (m, 1H), 3.81-3.66 (m, 5H), 3.15-3.08 (m, 1H), 2.98-2.90 (m, 3H), 2.65 (s, 3H), 2.45 (s, 3H), 2.26 (s, 1H), 2.06 (s, 1H), 1.93 (s, 2H), 1.82 (d, J=12.8 Hz, 2H), 1.75-1.64 (m, 1H), 1.00 (d, J=6.4 Hz, 3H).

LC-MS [column: C₁₈; column size: 4.6×50 mm; mobile phase: B (ACN) A (0.02% NH₄OAc); gradient (B %)]: Rt=3.701 min, MS Calcd.: 492, MS Found: 493 [M+H]⁺.

### Isomer 2:

Chiral HPLC [column: Superchiral S-AD ID; 0.46 cm×15 cm; Phase: CO₂/MeOH/DEA=60/40/0.05; Flow rate: 3.0 ml/min; Wave length: UV 254 nm; Temperature: 35° C.]: Rt=2.655 min.

¹H NMR (CDCl₃, 400 MHz): δ 8.81 (s, 1H), 8.05 (s, 1H), 7.49 (s, 1H), 6.95 (s, 1H), 4.78 (t, J=5.6 Hz, 2H), 4.76-4.72 (m, 2H), 4.32-4.30 (m, 2H), 4.09-3.99 (m, 2H), 3.79-3.66 (m, 4H), 3.29 (s, 1H), 3.17-3.08 (m, 2H), 2.97-2.91 (m, 1H), 2.81-2.78 (m, 1H), 2.70-2.61 (m, 2H), 2.65 (s, 3H), 2.45 (s, 3H), 2.13-2.07 (m, 1H), 1.95-1.92 (m, 2H), 1.77 (d, J=12.8 Hz, 1H), 1.07 (d, J=6.4 Hz, 3H).

Chiral HPLC [column: Superchiral S-AD ID; 0.46 cm×15 cm; Phase: CO₂/MeOH/DEA=60/40/0.05; Flow rate: 3.0 ml/min; Wave length: UV 254 nm; Temperature: 35° C.]: Rt=3.703 min, MS Calcd.: 492, MS Found: 493 [M+H]⁺.

### Isomer 3:

Chiral HPLC [column: Superchiral S-AD ID; 0.46 cm×15 cm; Phase: CO₂/MeOH/DEA=60/40/0.05; Flow rate: 3.0 ml/min; Wave length: UV 254 nm; Temperature: 35° C.]: Rt=3.594 min.

¹H NMR (CDCl₃, 400 MHz): δ 8.80 (s, 1H), 8.06 (s, 1H), 7.50 (s, 1H), 6.95 (s, 1H), 4.83-4.77 (m, 2H), 4.69-4.60 (m, 2H), 4.33-4.30 (m, 2H), 4.08-4.05 (m, 1H), 3.80-3.66 (m, 5H), 3.14-3.08 (m, 1H), 2.98-2.90 (m, 3H), 2.65 (s, 3H), 2.45 (s, 3H), 2.26 (s, 1H), 2.06 (s, 1H), 1.93 (s, 2H), 1.82 (d, J=12.8 Hz, 2H), 1.73-1.64 (m, 1H), 1.00 (d, J=6.4 Hz, 3H).

LC-MS [column: C₁₈; column size: 4.6×50 mm; mobile phase: B (ACN) A (0.02% NH₄OAc); gradient (B %)]: Rt=3.703 min, MS Calcd.: 492, MS Found: 493 [M+H]⁺.

### Isomer 4:

Chiral HPLC [column: Superchiral S-AD ID; 0.46 cm×15 cm; Phase: CO₂/MeOH/DEA=60/40/0.05; Flow rate: 3.0 ml/min; Wave length: UV 254 nm; Temperature: 35° C.]: Rt=5.014 min.

¹H NMR (CDCl₃, 400 MHz): δ 8.81 (s, 1H), 8.05 (s, 1H), 7.50 (s, 1H), 6.95 (s, 1H), 4.77 (t, J=6.0 Hz, 2H), 4.72-4.67 (m, 2H), 4.36-4.25 (m, 2H), 4.09-3.99 (m, 2H), 3.79-3.66 (m, 4H), 3.29 (s, 1H), 3.17-3.14 (m, 2H), 2.97-2.91 (m, 1H), 2.81-2.78 (m, 1H), 2.67-2.64 (m, 1H), 2.65 (s, 3H), 2.45 (s, 3H), 2.09-1.95 (m, 4H), 1.78 (d, J=12.8 Hz, 1H), 1.09 (d, J=6.4 Hz, 3H).

LC-MS [column: C₁₈; column size: 4.6×50 mm; mobile phase: B (ACN) A (0.02% NH₄OAc); gradient (B %)]: Rt=3.704 min, MS Calcd.: 492, MS Found: 493 [M+H]⁺.

### Example C-1

### (S)-4-(2-Methoxy-6-(5-methyl-6-(1-(oxetan-3-yl)piperidin-4-yl)-1H-indazol-1-yl)pyrimidin-4-yl)-3-methylmorpholine

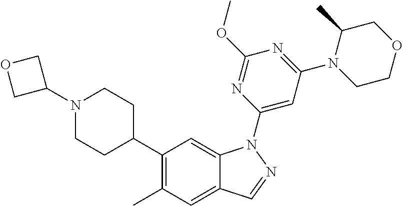 [SMILES: COc1nc(N2CCOC[C@@H]2C)cc(-n2ncc3cc(C)c(C4CCN(C5COC5)CC4)cc32)n1]

To a solution of (S)-4-(2-methoxy-6-(5-methyl-6-(piperidin-4-yl)-1H-indazol-1-yl)pyrimidin-4-yl)-3-methylmorpholine (115 mg, 0.27 mmol), oxetan-3-one (98 mg, 1.36 mmol) and NaBH₃CN (34 mg, 0.54 mmol) in DCM (5 mL) was added AcOH (1 drop). The mixture was stirred at room temperature for 18 hours. The mixture was concentrated. The residue was purified by prep-HPLC: (xbridge C18 SN.24813505811206 waters, gilson-1 X-bridge C₁₈ 5 μm 19×150 mm 40-80% B, A: H₂O (0.1% NH₄HCO₃), B: ACN, UV: 254 nm, Flowrate: 15 ml/min, GT: 12 mins) to give the title compound (20 mg, 15%) as a white solid.

¹HNMR (400 MHz, CD₃OD): δ 8.72 (s, 1H), 8.13 (s, 1H), 7.56 (s, 1H), 6.85 (s, 1H), 4.73-4.64 (m, 4H), 4.48-4.42 (m, 1H), 4.10-3.98 (m, 5H), 3.82-3.71 (m, 2H), 3.61-3.55 (m, 2H), 3.36 (s, 1H) 2.97-2.90 (m, 3H), 2.45 (s, 3H), 2.09-2.03 (m, 2H), 1.90-1.85 (m, 4H), 1.32 (d, J=6.8 Hz, 3H).

LC-MS [column: C₁₈; column size, 50×4.6 mm; mobile phase: B (ACN): A (0.02% NH₄OAc); gradient (B %) in 6 min]: Rt=4.006 min; MS Calcd.: 478, MS Found: 479 [M+H]⁺.

### Examples C-2 and C-3

Examples C-2 and C-3 were prepared by refluxing the indazole, iodo compound and amine under N₂, in the presence of CuI and K₃PO₄.

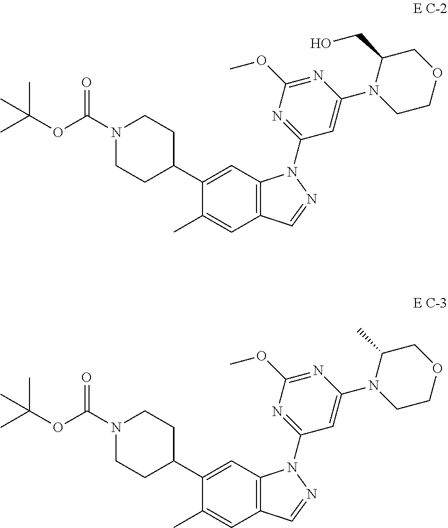 [SMILES: COc1nc(N2CCOC[C@@H]2CO)cc(-n2ncc3cc(C)c(C4CCN(C(=O)OC(C)(C)C)CC4)cc32)n1 ; COc1nc(N2CCOC[C@H]2C)cc(-n2ncc3cc(C)c(C4CCN(C(=O)OC(C)(C)C)CC4)cc32)n1]

| | Iodo | | | ¹HNMR (400 MHz, | |
|---|---|---|---|---|---|
| Indazole | compound | Amine | Product | CDCl₃): | LC-MS* |
| 5-methyl- | (S)-(4-(6- | N,N′- | (S)-tert-Butyl | δ 8.74 (s, 1H), | Rt = 4.005 min; |
| 6-(1- | iodo-2- | dimethylethylene- | 4-(1-(6-(3- | 8.06 (s, 1H), | MS |
| (oxetan- | methoxypyrimidin- | diamine | (hydroxymethyl)morpholino)- | 7.51 (s, 1H), 6.87 (s, | Calcd.: 494, |
| 3- | 4- | | 2- | 1H), 4.69 (d, J = 6.8 Hz, | MS Found: |
| yl)piperidin- | yl)morpholin- | | methoxypyrimidin- | 4H), | 495 [M + H]⁺ |
| 4-yl)- | 3- | | 4-yl)-5- | 4.61-4.51 (m, 1H), | |
| 1H- | yl)methanol | | methyl-1H- | 4.14 (s, 3H), | |
| indazole | | | indazol-6- | 4.10-3.97 (m, | |
| | | | yl)piperidine- | 4H), | |
| | | | 1-carboxylate | 3.71-3.53 (m, 3H), | |
| | | | (E C-2) | 3.44-3.36 (m, 1H), | |
| | | | | 2.95-2.92 (m, | |
| | | | | 2H), | |
| | | | | 2.87-2.80 (m, 1H), 2.46 (s, | |
| | | | | 3H), | |
| | | | | 2.25-2.20 (m, 1H), | |
| | | | | 2.04-1.99 (m, 2H), | |
| | | | | 1.94-1.85 (m, | |
| | | | | 4H). | |
| 5-methyl- | (R)-4-(6- | N,N′- | (R)-tert-Butyl | δ 8.77 (s, 1H), | Rt = 4.300 min; |
| 6-(1- | iodo-2- | dimethylcyclohexane- | 4-(1-(2- | 8.06 (s, 1H), | MS |
| (oxetan- | methoxypyrimidin- | 1,2- | methoxy-6-(3- | 7.51 (s, 1H), 6.80 (s, | Calcd.: 478, |
| 3- | 4- | diamine | methylmorpholino)pyrimidin- | 1H), 4.69 (d, J = 6.8 Hz, | MS Found: |
| yl)piperidin- | yl)-3- | | 4-yl)-5- | 4H), | 479 [M + H]⁺. |
| 4-yl)- | methylmorpholine | | methyl-1H- | 4.49-4.43 (m, 2H), | |
| 1H- | | | indazol-6- | 4.14 (s, 3H), | |
| indazole | | | yl)piperidine- | 4.02 (d, J = 12 Hz, | |
| | | | 1-carboxylate | 1H), | |
| | | | (E C-3) | 3.71-3.62 (m, 2H), | |
| | | | | 3.59-3.52 (m, 2H), | |
| | | | | 2.94-2.87 (m, | |
| | | | | 1H), 2.93 (d, J = 10.8 Hz, | |
| | | | | 2H), | |
| | | | | 2.86-2.79 (m, | |
| | | | | 1H), 2.45 (s, 3H), | |
| | | | | 2.01 (t, J = 15.6 Hz, | |
| | | | | 2H), | |
| | | | | 1.84-1.83 (m, 4H), | |
| | | | | 1.34 (d, J = 6.8 Hz, | |
| | | | | 3H). | |

*Phenomenex Kinetex 5 μm EVO C18, 50 * 4.6 mm; mobile phase: B (ACN): A (0.02% NH₄OAc); gradient (B %) in 6 min..

### Example C-4

### (R)-(4-(2-Methoxy-6-(5-methyl-6-(1-(oxetan-3-yl)piperidin-4-yl)-1H-indazol-1-yl)pyrimidin-4-yl)morpholin-3-yl)methanol

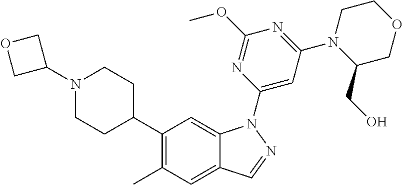 [SMILES: COc1nc(N2CCOC[C@H]2CO)cc(-n2ncc3cc(C)c(C4CCN(C5COC5)CC4)cc32)n1]

To a mixture of (R)-(4-(2-methoxy-6-(5-methyl-6-(piperidin-4-yl)-1H-indazol-1-yl)pyrimidin-4-yl)morpholin-3-yl)methanol (40 mg, 0.091 mmol) and oxetan-3-one (26 mg, 0.37 mmol) in DCM (2 mL)/MeOH (2 mL) was added AcOH/DCM solution (1 drop, from 1 drop HOAc in 1 mL DCM) and NaBH₃CN (12 mg, 0.18 mmol). The mixture was stirred at room temperature overnight. The mixture was concentrated. The residue was purified by pre-TLC (DCM/MeOH=20/1) to give the title compound (17 mg) as a white solid.

¹HNMR (400 MHz, CDCl₃): δ 8.74 (s, 1H), 8.06 (s, 1H), 7.51 (s, 1H), 6.87 (s, 1H), 4.69 (d, J=6.8 Hz, 4H), 4.61-4.51 (m, 1H), 4.14 (s, 3H), 4.10-3.97 (m, 4H), 3.71-3.53 (m, 3H), 3.44-3.36 (m, 1H), 2.95-2.92 (m, 2H), 2.87-2.80 (m, 1H), 2.46 (s, 3H), 2.25-2.20 (m, 1H), 2.04-1.99 (m, 2H), 1.94-1.85 (m, 4H).

LC-MS [Phenomenex Kinetex 5 μm EVO C₁₈, 50×4.6 mm; mobile phase: B (ACN): A (0.02% NH₄OAc); gradient (B %) in 6 min]: Rt=4.106 min; MS Calcd.: 494, MS Found: 495 [M+H]⁺.

### Examples C-5 and C-6

### (4-(2-Methoxy-6-(5-methyl-6-(1-(oxetan-3-yl)piperidin-4-yl)-1H-indazol-1-yl)pyrimidin-4-yl)-2-methylmorpholin-3-yl)methanol

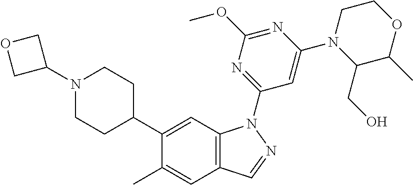 [SMILES: COc1nc(N2CCOC(C)C2CO)cc(-n2ncc3cc(C)c(C4CCN(C5COC5)CC4)cc32)n1]

To a solution of (4-(2-methoxy-6-(5-methyl-6-(piperidin-4-yl)-1H-indazol-1-yl)pyrimidin-4-yl)-2-methylmorpholin-3-yl)methanol (920 mg, 2.04 mmol), oxetan-3-one (734 mg, 10.2 mmol) and NaBH₃CN (257 mg, 4.08 mmol) in DCM (10 mL) was added 3 drops of AcOH. The mixture was stirred at room temperature for 14 hours. The reaction mixture was concentrated. The residue was purified by silica gel chromatography column (DCM/MeOH=50/1) to give the title compound (417 mg, 40%) as a yellow solid.

LCMS [column: C₁₈; column size: 4.6×30 mm 5 μm; Dikwa Diamonsil plus; mobile phase: B (ACN): A1 (0.02% NH₄OAc+5% ACN); gradient (B %) in 2.5 mins. 5-95-POS; flow rate: 1.5 ml/min]: Rt=1.59 min; MS Calcd.: 508, MS Found: 509 [M+H]⁺.

The mixture (4-(2-methoxy-6-(5-methyl-6-(1-(oxetan-3-yl)piperidin-4-yl)-1H-indazol-1-yl)pyrimidin-4-yl)-2-methylmorpholin-3-yl)methanol (389 mg) was separated by chiral-HPLC to afford isomer 1 (110 mg, 28%) and isomer 2 (130 mg, 33%).

### Chiral Prep-HPLC:

Column: Superchiral S-AD, 2 cm I.D.×25 cm, 5 μm; Phase: CO₂/IPE/NH₃H₂O=60/40/0.05; Flow rate: 30 ml/min; Wave length: 254 nm.

### Example C-5 (Isomer 1)

Chiral HPLC [column: Superchiral S-AD ID; 0.46 cm×15 cm; Phase: CO₂/IPA/DEA=60/40/0.05; Flow rate: 3.0 ml/min; Wave length: UV 254 nm; Temperature: 35° C.]: Rt=4.625 min.

LC-MS [column: C₁₈; column size: 4.6×50 mm; mobile phase: B (ACN) A (0.2% TFA); gradient (B %)]: Rt=3.586 min, MS Calcd.: 508, MS Found: 509 [M+H]⁺.

¹H NMR (CDCl₃, 400 MHz): δ 8.76 (s, 1H), 8.07 (s, 1H), 7.51 (s, 1H), 6.81 (s, 1H), 4.69 (t, J=6.4 Hz, 4H), 4.15 (s, 3H), 4.08-4.05 (m, 1H), 3.78-3.54 (m, 5H), 3.49 (s, 1H), 3.29 (br s, 1H), 2.95-2.92 (m, 2H), 2.88-2.79 (m, 1H), 2.46 (s, 3H), 2.05-1.82 (m, 7H), 1.16 (d, J=6.8 Hz, 3H).

### Example C-6

### Isomer 2:

Chiral HPLC [column: Superchiral S-AD ID; 0.46 cm×15 cm; Phase: CO₂/IPA/DEA=60/40/0.05; Flow rate: 3.0 ml/min; Wave length: UV 254 nm; Temperature: 35° C.]: Rt=5.

177 min.

LC-MS [column: C₁₈; column size: 4.6×50 mm; mobile phase: B (ACN) A (0.2% TFA); gradient (B %)]: Rt=3.587 min, MS Calcd.: 508, MS Found: 509 [M+H]⁺.

¹H NMR (CDCl₃, 400 MHz): b 8.76 (s, 1H), 8.07 (s, 1H), 7.51 (s, 1H), 6.81 (s, 1H), 4.69 (t, J=6.8 Hz, 4H), 4.15 (s, 3H), 4.10-4.05 (m, 1H), 3.80-3.54 (m, 5H), 3.49 (s, 1H), 3.30 (br s, 1H), 2.95-2.92 (m, 2H), 2.86-2.81 (m, 1H), 2.46 (s, 3H), 2.04-1.80 (m, 7H), 1.16 (d, J=6.8 Hz, 3H).

### F. Assays and Data

As stated above, the compounds of present invention are LRRK2 kinase inhibitors, and may be useful in the treatment of diseases mediated by LRRK2. The biological activities and/or properties of the compounds of present invention can be determined using any suitable assay, including assays for determining the activity of a candidate compound as a LRRK2 kinase inhibitor, as well as tissue and in vivo models.

### 1. Assays

a. Full Length G2019 Human LRRK2 Inhibition Mass Spectrometry Assay

This assay for Leucine Rich Repeat Kinase 2 (LRRK2) inhibition is based on the direct measurement of the peptide ‘LRRKtide’ (LRRKtide: RLGRDKYKT*LRQIRQ and “*” refers to the site of phosphorylation.) and phosphorylated ‘LRRKtide’ using a high throughput RapidFire mass spectrometry assay. Inhibitors are compounds that reduce the conversion of LRRKtide to phospho-LRRKtide.

### Human G2019 LRRK2 Plasmid Preparation

Primers used for PCR cloning:
pHTBV-F: SEQ ID No: 1
LRRK2 wt-F1: SEQ ID No: 2
LRRK2 wt-R¹: SEQ ID No: 3
LRRK2 wt-F2: SEQ ID No: 4
LRRK2 wt-R²: SEQ ID No: 5
LRRK2 wt-F3: SEQ ID No: 6
pHTBV-R: SEQ ID No: 7

pHTBV1-N-Flag-hu LRRK2 was generated by PCR amplifying the full length LRRK2 sequence with N terminal Flag tag from pcDNA3.1(+)_Human_LRRK2 (NCBI Reference Sequence: NP_940980.3) with the primers described above, and cloned into pHTBV1mcs3 vector between BamHI and KpnI sites.

The G2019 full length Flag-LRRK2 coding sequence is SEQ ID No: 8.

The translated amino acid sequence for human G2019 full length N terminal flag tagged LRRK2 protein is SEQ ID No: 9.

### Insect Cell Cultures

Sf9 insect cells (Invitrogen Life Technologies, Carlsbad, Calif.) were maintained at 27° C. in SF 900 II SFM in 500-ml shaker flasks (Erlenmeyer, Corning). The cells were maintained in exponential growth phase and subcultured twice per week. For larger volumes, cells were grown in 2-liter shaker flasks (Erlenmeyer, Corning) while being agitated with 120 rpm at 27° C. incubator shaker.

### Generation of the BacMam Virus

To generate the recombinant BacMam virus, DH10Bac competent cells (10361-012, Invitrogen) were transformed by the genotypically normal human LRRK2 BacMam plasmid to generate the recombinant baculovirus DNA. The Sf9 insect cells were co-transfected with the mixture of recombinant baculovirus DNA and cellfectin (10362-100, Invitrogen). After 4 h of incubation at 27° C., the transfection media was replaced with Sf-900 III SFM medium containing 5% HI FBS (Ser. No. 10/100,147, Invitrogen). The cells were further incubated for 4 days. The infected cell culture medium containing the baculovirus (P0 virus stock) was collected and amplified by further infecting the 200 ml Sf9 cells via 200-300 ul P0.

### Quantification of BacMam Viral Titre by BacPAKRapid Titer

The viral titre, measured as plaque forming unit (pfu)/ml was determined using BacPAK Papid Titer Kit (631406, Clontech) according to the manufacturer's protocol. The Sf9 cells seeded in 96-well plate with 3×10⁵ cells per well were incubated with serial dilution of the viral stocks for 1 h at 27° C., 50 μl methyl cellulose overlay was added to each well followed by 43˜47 h incubation. The cells were then fixed in 4% paraformaldehyde (PFA). After blocking the cells with diluted normal goat serum, Mouse anti-gp64 antibody was added to the cells. After 30 min incubation, the cells were washed with phosphate buffered saline containing 0.2% Triton-X100 (PBST) and incubated for another 30 min with goat anti-mouse antibody/HRP conjugate. This was followed by blue peroxidase substrate which detects the single infected cells and foci of infected cells by their dark blue color.

### Protein Expression & Purification

a) Expression of Flag Tagged Full Length G2019 Human LRRK2

HEK293 6E cells were incubated in a 37° C. incubator with a humidified atmosphere of 5% CO₂ on an orbital shaker rotating at 110 rpm. On the day of transduction, the cell viability was higher than 98% and the cell density was in the range of 1×10⁶˜1.5×10⁶ cells/ml. HEK293 6E cells were centrifuged at 1,000 rpm for 10 min, and then the cells were resuspended in fresh Freestyle 293 expression medium (Invitrogen: 12338) with 0.1% F-68(Invitrogen: 24040-032) but without antibiotics (G418) at density of 1×10⁶ cells/ml. BacMam virus with Flag-hu LRRK2 (genotypically normal) gene was centrifuged at 40,000 g for 2 hours, then resuspended in fresh Freestyle 293 expression medium. The resuspended virus was added into the cells in at MOI of 10. The cells were incubated in a 37° C. incubator with a humidified atmosphere of 5% CO₂ in air on an orbital shaker rotating at 110 rpm. Cultures were harvested at approximately 48 hours post-transduction by centrifugation at 4,000 rpm for 20 min and pellets were frozen for purification.

b) Purification of Flag Tagged Full Length G2019 Human LRRK2

The cell pellet was resuspended in (20 mL/liter cell culture) lysis buffer (50 mM TrisHCl pH7.5 at 4° C., 500 mM NaCl, 0.5 mM EDTA, 0.1% TritonX-100, 10% glycerol, freshly add 2 mM DTT), with protease inhibitors (Roche: 04693132001) and benzonase (Merck Millipore: 70746-3CN) at recommended concentration suggested by suppliers. The suspended cells were lysed by sonication on ice for 30 min (2 secs on/4 sec off, 20% amplitude), and centrifuged at 10,000 rpm for 30 minutes at 4° C. The supernatant was incubated with 1 mL per litre of cell culture of anti-Flag magnetic beads (Sigma-Aldrich: M8823) at 4° C. for 3 hours, then the beads were washed by 5 mL (5 column volume) binding buffer (50 mM Tris pH7.5@ 4 C, 500 mM NaCl, 0.5 mM EDTA, 0.1% TritonX-100, 10% glycerol, freshly add 2 mM DTT) for three times. The Flag tagged LRRK2 proteins were eluted by Elution buffer (50 mM Tris pH7.5@ 4 C, 500 mM NaCl, 0.5 mM EDTA, 0.1% Triton X-100, 10% glycerol, freshly add 2 mM DTT, 250 ug/ml Flag peptide (Sigma-Aldrich:F3290)) at 4° C. for 2 hours. Flag peptide was removed by Zeba Spin Desalting Columns, 7K MWCO (Thermo-Fisher: 89893) and the buffer of eluted LRRK2 proteins was exchanged into Storage Buffer (50 mM Tris pH7.5@4 C, 150 mM NaCl, 0.5 mM EDTA, 0.02% Triton X-100, 2 mM DTT and 50% Glycerol) using Amicon Ultra Centrifugal Filter Units (100 kD) (Merck: UFC910096). Fractions containing LRRK2 proteins were pooled, aliquoted and stored at −80° C. Protein concentration was determined by Bradford protein assay, and protein purity was analyzed by NuPAG Novex 4-12% Bis-Tris Protein Gels (Invitrogen: NP0322BOX).

### Assay Protocol

1) A 10 mM test compound was dissolved in 100% DMSO and serially diluted 1 in 4. 100 nL of this dilution series was then added to a 384 well, v bottom polypropylene plate, excluding columns 6 and 18. 100 nL of DMSO was added to columns 6 and 18 as controls wells. Assay dilution gave a top final assay concentration of test compound of 100 μM
2) 50 μl of 1% formic acid in laboratory grade water was added to column 18 using a multidrop combi dispenser to act as a pre stopped assay control.
3) 5 μl of ‘enzyme solution’ containing 50 nM of purified recombinant Full length Flag-LRRK2 in assay buffer (50 mM Hepes (pH 7.2), 10 mM MgCl2, 150 mM NaCl, 5% glycerol, 0.0025% triton X-100 and 1 mM DTT) was added to all wells using a multidrop combi dispenser, giving a final assay concentration of 25 nM LRRK2 enzyme. This resulted in column 6 (enzyme plus DMSO) giving 0% inhibition and column 18 giving 100% inhibition (pre stopped control). Test plates were then incubated for 30 minutes at room temperature.
4) 5 μl ‘substate solution’ containing 50 uM LRRKtide peptide substrate and 4 mM ATP was added to all wells of the plate using a multidrop combi dispenser giving a final assay concentration of 25 uM LRRKtide and 2 mM ATP. Test plates were then incubated for 1 hour at room temperature. (Incubation may vary depending on rate and linearity of reaction with different enzyme batches).
5) 50 μl of 1% formic acid in laboratory grade water was added to all wells (minus column 18) to quench the reaction, and plates were centrifuged at 3000 rpm for 10 minutes. Test plates were then analysed on an Agilent RapidFire High Throughput solid phase extraction system coupled to AB Sciex API 4000 triple quadropole mass spectrometer with the following setting:
   RapidFire settings:
   Sip Height=2 mm, Aspirate=500 ms, Load time=3000 ms, Elution time=3000 ms, Requilibration=500 ms.
   Flow rates: pump 1=1.5 mL/min, pump 2 1.25 mL/min pump 3=0.8 mL/min Mass Spectrometer Settings:
   LRRKtide Detection settings: Q1 mass 644.8 Da, Q3 mass 638.8, declustering potential 76 volts, collision energy 37 volts, CXP 34 volts.
   Phospho-LRRKtide Detection settings: Q1 mass 671.4 Da, Q3 mass 638.8, Declustering potential 76 volts, Collision energy 37 volts, CXP 34 volts.
   A C4 cartridge was used and running buffers were: A (aqueous) 0.1% formic acid in water B (organic) 0.1% formic acid, 80% acetonitrile, 20% water.
   Collision gas: 12, Curtain gas: 25, Ion Source gas (1): 60, Ion Source gas (2): 60, Ion Spray Voltage: 5500, Temperature: 600, Interface Heater: ON.
   Resolution Q1: low, Resolution Q3: low.
6) Data was analysed using ActivityBase software (IDBS). A percent conversion from LRRKtide to Phospho-LRRKtide was calculated using the following formula:

% conversion=(Phospho-LRRKtide product peak area/(Phospho-LRRKtide product peak area+LRRKtide substrate peak area))*100

b. Recombinant Cellular LRRK2 AlphaScreen Assay

To determine the activity of compounds against LRRK2 kinase activity in cells, the observed LRRK2 kinase-dependent modulation of LRRK2 Ser 935 phosphorylation (Dzamko et al., 2010, Biochem. J. 430: 405-413) was utilized to develop a quantitative 384 well plate-based immunoassay of LRRK2 Ser935 phosphorylation in the human neuroblastoma cell line SH-SY5Y, engineered to over-express recombinant full length LRRK2 protein.

A BacMam virus expressing full length recombinant LRRK2 was purchased from Invitrogen and amplified by inoculation of SF-9 cells at MOI 0.3 for 4-5 days in Sf-900 III SFM medium supplemented with 3% fetal bovine serum. Infected cell cultures were then centrifuged at 2000 g for 20 minutes, viral supernatant titer determined by anti-gp64 plaque assay and stored at 4° C.

Affinity-purified anti-phospho LRRK2 Ser935 sheep polyclonal antibody (Dzamko et al., 2010, Biochem. J. 430: 405-413) was biotinylated by standard methods (PerkinElmer). Anti-LRRK2 rabbit polyclonal antibody was purchased from Novus Biologicals. AlphaScreen Protein A IgG Kit (including acceptor and donor beads) was purchased from Perkin Elmer.

SH-SY5Y cells were grown in DMEM/F12 medium with 10% dialysed fetal bovine serum and harvested by treatment with 0.5% trypsin-EDTA for 5 minutes at 37° C. followed by centrifugation at 1000 rpm for 4 minutes. The cell pellet was resuspended in Opti-MEM reduced serum media (Invitrogen) at 200,000 cells/ml and mixed with the BacMam LRRK2 virus at MOI=50. 50 μl cell solutions were then dispensed to each well of a 384-well plate and incubated at 37° C., 5% CO₂ for 24 hours.

Serial dilutions of test compounds were prepared in Opti-MEM reduced serum media (Invitrogen) and 5.6 ul transferred from compound plate to cell assay plate to achieve a top final assay concentration of 10 uM. DMSO was used in certain wells as controls. Cells were incubated at 37° C., 5% CO₂ for 60 minutes. The medium was then removed and cells lysed by addition of 20 ul cell lysis buffer (Cell Signaling Technology) and incubation at 4° C. for 20 minutes. 10 ul of antibody/acceptor bead mix [(1/1000 biotinylated-pS935 LRRK2 antibody, 1/1000 total-LRRK2 antibody, 1/100 Acceptor beads in AlphaScreen detection buffer (25 mM Hepes (pH 7.4), 0.5% Triton X-100, 1 mg/ml Dextran 500 and 0.1% BSA)] was then added to each well and plates incubated for 2 hours at ambient temperature in the dark. 10 μl of donor beads solution (1/33.3 donor beads in AlphaScreen detection buffer) was then added to each well. Following incubation for a further 2 hours at ambient temperature in the dark, plates were read on an EnVision™ plate reader at emission 520-620 nm with excitation 680 nm. Dose response curve data was based on sigmoidal dose-response model.

c. FASSIF Solubility Assay

Compound solubility may be evaluated in the fasted state simulated intestinal media (FaSSIF) at pH 6.5. Certain amount of test compound was admixed with certain volume of FaSSIF to prepare a suspension of about 1 mg/ml. The suspension was incubated at 37° C. in the water bath shaker for 24 hours. At the 4^{th} and 24^{th} hour, the suspension was centrifugated at 14K rpm for 15 minutes. 100 μl of the supernatant was withdrawn and diluted with the same volume of 50% acetonitrile water solution and analysed with UPLC (Ultra performance Liquid Chromatography). FaSSIF solubility was calculated based on the peak area of the test compound.

The FaSSIF (170 ml) preparation 100 mg of lecithin and 274 mg (anhyd equiv) of NaTaurocholate were dissolved in about 150 ml of pH 6.5 buffer. The solution was made to the volume of 170 ml with the pH 6.5 buffer.

The pH 6.5 buffer solution (1 L) preparation 4.083 g KH₂PO₄ and 7.456 g KCl were dissolved in 800 ml of water, with 100 ml 0.1 M NaOH subsequently added. The solution was made to the volume of 1 L with water. The pH value of the buffer solution was measured and adjusted to be 6.50±0.1.

Standard solutions for UPLC calibration and solubility calculation 2 μM, 20 μM and 200 μM DMSO (50% ACN water) solutions.

UPLC Method and Parameter
Instrument: Waters ACQUITY UPLC System
Column: Waters ACQUITY UPLC BEH C18 (1.7 μm, 2.1×50 mm)
Mobile phase: A: 0.1% TFA in water/B: 0.1% TFA in CAN
Gradient: 0 min (A 95%/B 5%), 2 min (A 5%/B 95%), 2.5 min (A 5%/B 95%), 2.6 min (A 95%/B 5%), 3 min (A 95%/B 5%)
Flow rate: 0.8 mL/min; column temperature: 40° C.; injection volume: 1.0 μL; UV detection: 280 nm d. CLND Solubility Assay

Kinetic solubility of a compound may be evaluated by the CLND (ChemiLuminescent Nitrogen Detection) solubility assay, based on known protocols (see, e.g., Bhattachar S. N.; Wesley J. A.; Seadeek C., Evaluation of the Chemiluminescent Nitrogen Detector for Solubility Determinations to Support Drug Discovery, J. Pharm. Biomed. Anal. 2006 (41): 152-157; Kestranek A, Chervenek A, Logenberger J, Placko S. Chemiluminescent Nitrogen Detection (CLND) to Measure Kinetic Aqueous Solubility, Curr Protoc Chem Biol., 2013, 5(4):269-80). Typically, 5 μl of 10 mM DMSO stock solution of the test compound was diluted to 100 μl with pH7.4 phosphate buffered saline, equilibrated for 1 hour at room temperature, filtered through Millipore MultiscreenHTS-PCF filter plates (MSSL BPC). The filtrate is quantified by suitably calibrated flow injection Chemi-Luminescent Nitrogen Detection.

### 2. Assay Data

Compounds of Examples E A-1-A-4 were tested in the recombinant cellular LRRK2 AlphaScreen assay and exhibited a plC50 of ≥6.5. The compound of Example A-1 exhibited a plC50 of 6.7 in the recombinant cellular LRRK2 AlphaScreen assay.

Compounds of Examples E A-3 and E A-4 were tested in the recombinant cellular LRRK2 AlphaScreen assay and exhibited a plC50 of ≥7.5. The compound of Example A-3 exhibited a plC50 of 7.9 in the recombinant cellular LRRK2 AlphaScreen assay.

Compounds of Examples E A-1 and E A-2 were tested in the full length G2019 human LRRK2 Inhibition Mass Spectrometry assay and exhibited a plC50 of ≥7.0.

Compounds of Examples E B-1-E B-4 and E B-6-E B-8 were tested in the recombinant cellular LRRK2 AlphaScreen assay and exhibited a plC50 of ≥6.5 as follows:

| Example | pIC50 |
|---|---|
| B-1 | 7.3 |
| B-2 | 7.1 |
| B-3 | 7.3 |
| B-4 | 6.5 |
| B-6 | 7.2 |
| B-7 | 7.1 |
| B-8 | 7 |

Compounds of Examples E B-7 and E B-8 were tested in the full length G2019 human LRRK2 Inhibition Mass Spectrometry assay and exhibited a plC50 of ≥7.0.

Compounds of Examples E C-2 and E C-3 were tested in the recombinant cellular LRRK2 AlphaScreen assay and exhibited a plC50 of ≥7.0.

### 3. Sequence Listing

Primers used for PCR cloning of Human G2019 LRRK2 plasmids preparation: pHTBV-
F
SEQ ID NO: 1
5′-GATCTCGACGGGCGCGGATCCACCATGGATTACAAGGATGACGACGAT-3′
Primers used for PCR cloning of Human G2019 LRRK2 plasmids preparation: LRRK2
wt-F1
SEQ ID NO: 2
5′-CATGGATTACAAGGATGACGACGATAAGATGGCTAGTGGCAGCTGTCAG-3′
Primers used for PCR cloning of Human G2019 LRRK2 plasmids preparation: LRRK2
wt-R1
SEQ ID NO: 3
5′-GTTCACGAGATCCACTATTCAGTAAGAGTTCCACCAATTTGGGACTG-3′
Primers used for PCR cloning of Human G2019 LRRK2 plasmids preparation: LRRK2
wt-F2
SEQ ID NO: 4
5′-GAATAGTGGATCTCGTGAACAAG-3′
Primers used for PCR cloning of Human G2019 LRRK2 plasmids preparation: LRRK2
wt-R2
SEQ ID NO: 5
5′-GTCAGACAAACTGCTTGGAACCAGC-3′
Primers used for PCR cloning of Human G2019 LRRK2 plasmids preparation: LRRK2
wt-F3
SEQ ID NO: 6
5′-CTGGTTCCAAGCAGTTTGTCTGACCACAGGCCTGTGATAG-3′
Primers used for PCR cloning of Human G2019 LRRK2 plasmids preparation: pHTBV-
R
SEQ ID NO: 7
5′-GTTCTAGCCAAGCTTGGTACCCTATTACTCAACAGATGTTCGTCTC-3′
G2019 Full length Flag-LRRK2 coding sequence
SEQ ID NO: 8
atggattacaaggatgacgacgataagATGGCTAGTGGCAGCTGTCAGGGGTGCGAAGAGGACGAGGAAAC
TCTGAAGAAGTTGATAGTCAGGCTGAACAATGTCCAGGAAGGAAAACAGATAGAAACGCTGGTC
CAAATCCTGGAGGATCTGCTGGTGTTCACGTACTCCGAGCACGCCTCCAAGTTATTTCAAGGCAA
AAATATCCATGTGCCTCTGTTGATCGTCTTGGACTCCTATATGAGAGTCGCGAGTGTGCAGCAGG
TGGGTTGGTCACTTCTGTGCAAATTAATAGAAGTCTGTCCAGGTACAATGCAAAGCTTAATGGGA
CCCCAGGATGTTGGAAATGATTGGGAAGTCCTTGGTGTTCACCAATTGATTCTTAAAATGCTAAC
AGTTCATAATGCCAGTGTAAACTTGTCAGTGATTGGACTGAAGACCTTAGATCTCCTCCTAACTTC
AGGTAAAATCACCTTGCTGATACTGGATGAAGAAAGTGATATTTTCATGTTAATTTTTGATGCCAT
GCACTCATTTCCAGCCAATGATGAAGTCCAGAAACTTGGATGCAAAGCTTTACATGTGCTGTTTG
AGAGAGTCTCAGAGGAGCAACTGACTGAATTTGTTGAGAACAAAGATTATATGATATTGTTAAGT
GCGTTAACAAATTTTAAAGATGAAGAGGAAATTGTGCTTCATGTGCTGCATTGTTTACATTCCCTA
GCGATTCCTTGCAATAATGTGGAAGTCCTCATGAGTGGCAATGTCAGGTGTTATAATATTGTGGT
GGAAGCTATGAAAGCATTCCCTATGAGTGAAAGAATTCAAGAAGTGAGTTGCTGTTTGCTCCATA
GGCTTACATTAGGTAATTTTTTCAATATCCTGGTATTAAACGAAGTCCATGAGTTTGTGGTGAAAG
CTGTGCAGCAGTACCCAGAGAATGCAGCATTGCAGATCTCAGCGCTCAGCTGTTTGGCCCTCCT
CACTGAGACTATTTTCTTAAATCAAGATTTAGAGGAAAAGAATGAGAATCAAGAGAATGATGATGA
GGGGGAAGAAGATAAATTGTTTTGGCTGGAAGCCTGTTACAAAGCATTAACGTGGCATAGAAAGA
ACAAGCACGTGCAGGAGGCCGCATGCTGGGCACTAAATAATCTCCTTATGTACCAAAACAGTTTA
CATGAGAAGATTGGAGATGAAGATGGCCATTTCCCAGCTCATAGGGAAGTGATGCTCTCCATGC
TGATGCATTCTTCATCAAAGGAAGTTTTCCAGGCATCTGCGAATGCATTGTCAACTCTCTTAGAAC
AAAATGTTAATTTCAGAAAAATACTGTTATCAAAAGGAATACACCTGAATGTTTTGGAGTTAATGCA
GAAGCATATACATTCTCCTGAAGTGGCTGAAAGTGGCTGTAAAATGCTAAATCATCTTTTTGAAGG
AAGCAACACTTCCCTGGATATAATGGCAGCAGTGGTCCCCAAAATACTAACAGTTATGAAACGTC
ATGAGACATCATTACCAGTGCAGCTGGAGGCGCTTCGAGCTATTTTACATTTTATAGTGCCTGGC
ATGCCAGAAGAATCCAGGGAGGATACAGAATTTCATCATAAGCTAAATATGGTTAAAAAACAGTG
TTTCAAGAATGATATTCACAAACTGGTCCTAGCAGCTTTGAACAGGTTCATTGGAAATCCTGGGAT
TCAGAAATGTGGATTAAAAGTAATTTCTTCTATTGTACATTTTCCTGATGCATTAGAGATGTTATCC
CTGGAAGGTGCTATGGATTCAGTGCTTCACACACTGCAGATGTATCCAGATGACCAAGAAATTCA
GTGTCTGGGTTTAAGTCTTATAGGATACTTGATTACAAAGAAGAATGTGTTCATAGGAACTGGACA
TCTGCTGGCAAAAATTCTGGTTTCCAGCTTATACCGATTTAAGGATGTTGCTGAAATACAGACTAA
AGGATTTCAGACAATCTTAGCAATCCTCAAATTGTCAGCATCTTTTTCTAAGCTGCTGGTGCATCA
TICATTTGACTTAGTAATATTCCATCAAATGTCTTCCAATATCATGGAACAAAAGGATCAACAGTTT
CTAAACCTCTGTTGCAAGTGTTTTGCAAAAGTAGCTATGGATGATTACTTAAAAAATGTGATGCTA
GAGAGAGCGTGTGATCAGAATAACAGCATCATGGTTGAATGCTTGCTTCTATTGGGAGCAGATG
CCAATCAAGCAAAGGAGGGATCTTCTTTAATTTGTCAGGTATGTGAGAAAGAGAGCAGTCCCAAA
TTGGTGGAACTCTTACTGAATAGTGGATCTCGTGAACAAGATGTACGAAAAGCGTTGACGATAAG
CATTGGGAAAGGTGACAGCCAGATCATCAGCTTGCTCTTAAGGAGGCTGGCCCTGGATGTGGCC
AACAATAGCATTTGCCTTGGAGGATTTTGTATAGGAAAAGTTGAACCTTCTTGGCTTGGTCCTTTA
TTTCCAGATAAGACTTCTAATTTAAGGAAACAAACAAATATAGCATCTACACTAGCAAGAATGGTG
ATCAGATATCAGATGAAAAGTGCTGTGGAAGAAGGAACAGCCTCAGGCAGCGATGGAAATTTTTC
TGAAGATGTGCTGTCTAAATTTGATGAATGGACCTTTATTCCTGACTCTTCTATGGACAGTGTGTT
TGCTCAAAGTGATGACCTGGATAGTGAAGGAAGTGAAGGCTCATTTCTTGTGAAAAAGAAATCTA
ATTCAATTAGTGTAGGAGAATTTTACCGAGATGCCGTATTACAGCGTTGCTCACCAAATTTGCAAA
GACATTCCAATTCCTTGGGGCCCATTTTTGATCATGAAGATTTACTGAAGCGAAAAAGAAAAATAT
TATCTTCAGATGATTCACTCAGGTCATCAAAACTTCAATCCCATATGAGGCATTCAGACAGCATTT
CTTCTCTGGCTTCTGAGAGAGAATATATTACATCACTAGACCTTTCAGCAAATGAACTAAGAGATA
TTGATGCCCTAAGCCAGAAATGCTGTATAAGTGTTCATTTGGAGCATCTTGAAAAGCTGGAGCTT
CACCAGAATGCACTCACGAGCTTTCCACAACAGCTATGTGAAACTCTGAAGAGTTTGACACATTT
GGACTTGCACAGTAATAAATTTACATCATTTCCTTCTTATTTGTTGAAAATGAGTTGTATTGCTAAT
CTTGATGTCTCTCGAAATGACATTGGACCCTCAGTGGTTTTAGATCCTACAGTGAAATGTCCAACT
CTGAAACAGTTTAACCTGTCATATAACCAGCTGTCTTTTGTACCTGAGAACCTCACTGATGTGGTA
GAGAAACTGGAGCAGCTCATTTTAGAAGGAAATAAAATATCAGGGATATGCTCCCCCTTGAGACT
GAAGGAACTGAAGATTTTAAACCTTAGTAAGAACCACATTTCATCCCTATCAGAGAACTTTCTTGA
GGCTTGTCCTAAAGTGGAGAGTTTCAGTGCCAGAATGAATTTTCTTGCTGCTATGCCTTTCTTGC
CTCCTTCTATGACAATCCTAAAATTATCTCAGAACAAATTTTCCTGTATTCCAGAAGCAATTTTAAA
TCTTCCACACTTGCGGTCTTTAGATATGAGCAGCAATGATATTCAGTACCTACCAGGTCCCGCAC
ACTGGAAATCTTTGAACTTAAGGGAACTCTTATTTAGCCATAATCAGATCAGCATCTTGGACTTGA
GTGAAAAAGCATATTTATGGTCTAGAGTAGAGAAACTGCATCTTTCTCACAATAAACTGAAAGAGA
TTCCTCCTGAGATTGGCTGTCTTGAAAATCTGACATCTCTGGATGTCAGTTACAACTTGGAACTAA
GATCCTTTCCCAATGAAATGGGGAAATTAAGCAAAATATGGGATCTTCCTTTGGATGAACTGCAT
CTTAACTTTGATTTTAAACATATAGGATGTAAAGCCAAAGACATCATAAGGTTTCTTCAACAGCGA
TTAAAAAAGGCTGTGCCTTATAACCGAATGAAACTTATGATTGTGGGAAATACTGGGAGTGGTAA
AACCACCTTATTGCAGCAATTAATGAAAACCAAGAAATCAGATCTTGGAATGCAAAGTGCCACAG
TTGGCATAGATGTGAAAGACTGGCCTATCCAAATAAGAGACAAAAGAAAGAGAGATCTCGTCCTA
AATGTGTGGGATTTTGCAGGTCGTGAGGAATTCTATAGTACTCATCCCCATTTTATGACGCAGCG
AGCATTGTACCTTGCTGTCTATGACCTCAGCAAGGGACAGGCTGAAGTTGATGCCATGAAGCCTT
GGCTCTTCAATATAAAGGCTCGCGCTTCTTCTTCCCCTGTGATTCTCGTTGGCACACATTTGGAT
GTTTCTGATGAGAAGCAACGCAAAGCCTGCATGAGTAAAATCACCAAGGAACTCCTGAATAAGCG
AGGGTTCCCTGCCATACGAGATTACCACTTTGTGAATGCCACCGAGGAATCTGATGCTTTGGCAA
AACTTCGGAAAACCATCATAAACGAGAGCCTTAATTTCAAGATCCGAGATCAGCTTGTTGTTGGA
CAGCTGATTCCAGACTGCTATGTAGAACTTGAAAAAATCATTTTATCGGAGCGTAAAAATGTGCCA
ATTGAATTTCCCGTAATTGACCGGAAACGATTATTACAACTAGTGAGAGAAAATCAGCTGCAGTTA
GATGAAAATGAGCTTCCTCACGCAGTTCACTTTCTAAATGAATCAGGAGTCCTTCTTCATTTTCAA
GACCCAGCACTGCAGTTAAGTGACTTGTACTTTGTGGAACCCAAGTGGCTTTGTAAAATCATGGC
ACAGATTTTGACAGTGAAAGTGGAAGGTTGTCCAAAACACCCTAAGGGAATTATTTCGCGTAGAG
ATGTGGAAAAATTTCTTTCAAAGAAAAGGAAATTTCCAAAGAACTACATGTCACAGTATTTTAAGC
TCCTAGAAAAATTCCAGATTGCTTTGCCAATAGGAGAAGAATATTTGCTGGTTCCAAGCAGTTTGT
CTGACCACAGGCCTGTGATAGAGCTTCCCCATTGTGAGAACTCTGAAATTATCATCCGACTATAT
GAAATGCCTTATTTTCCAATGGGATTTTGGTCAAGATTAATCAATCGATTACTTGAGATTTCACCTT
ACATGCTTTCAGGGAGAGAACGAGCACTTCGCCCAAACAGAATGTATTGGCGACAAGGCATTTA
CTTAAATTGGTCTCCTGAAGCTTATTGTCTGGTAGGATCTGAAGTCTTAGACAATCATCCAGAGA
GTTTCTTAAAAATTACAGTTCCTTCTTGTAGAAAAGGCTGTATTCTTTTGGGCCAAGTTGTGGACC
ACATTGATTCTCTCATGGAAGAATGGTTTCCTGGGTTGCTGGAGATTGATATTTGTGGTGAAGGA
GAAACTCTGTTGAAGAAATGGGCATTATATAGTTTTAATGATGGTGAAGAACATCAAAAAATCTTA
CTTGATGACTTGATGAAGAAAGCAGAGGAAGGAGATCTCTTAGTAAATCCAGATCAACCAAGGCT
CACCATTCCAATATCTCAGATTGCCCCTGACTTGATTTTGGCTGACCTGCCTAGAAATATTATGTT
GAATAATGATGAGTTGGAATTTGAACAAGCTCCAGAGTTTCTCCTAGGTGATGGCAGTTTTGGAT
CAGTTTACCGAGCAGCCTATGAAGGAGAAGAAGTGGCTGTGAAGATTTTTAATAAACATACATCA
CTCAGGCTGTTAAGACAAGAGCTTGTGGTGCTTTGCCACCTCCACCACCCCAGTTTGATATCTTT
GCTGGCAGCTGGGATTCGTCCCCGGATGTTGGTGATGGAGTTAGCCTCCAAGGGTTCCTTGGAT
CGCCTGCTTCAGCAGGACAAAGCCAGCCTCACTAGAACCCTACAGCACAGGATTGCACTCCACG
TAGCTGATGGTTTGAGATACCTCCACTCAGCCATGATTATATACCGAGACCTGAAACCCCACAAT
GTGCTGCTTTTCACACTGTATCCCAATGCTGCCATCATTGCAAAGATTGCTGACTACGGCATTGC
TCAGTACTGCTGTAGAATGGGGATAAAAACATCAGAGGGCACACCAGGGTTTCGTGCACCTGAA
GTTGCCAGAGGAAATGTCATTTATAACCAACAGGCTGATGTTTATTCATTTGGTTTACTACTCTAT
GACATTTTGACAACTGGAGGTAGAATAGTAGAGGGTTTGAAGTTTCCAAATGAGTTTGATGAATTA
GAAATACAAGGAAAATTACCTGATCCAGTTAAAGAATATGGTTGTGCCCCATGGCCTATGGTTGA
GAAATTAATTAAACAGTGTTTGAAAGAAAATCCTCAAGAAAGGCCTACTTCTGCCCAGGTCTTTGA
CATTTTGAATTCAGCTGAATTAGTCTGTCTGACGAGACGCATTTTATTACCTAAAAACGTAATTGTT
GAATGCATGGTTGCTACACATCACAACAGCAGGAATGCAAGCATTTGGCTGGGCTGTGGGCACA
CCGACAGAGGACAGCTCTCATTTCTTGACTTAAATACTGAAGGATACACTTCTGAGGAAGTTGCT
GATAGTAGAATATTGTGCTTAGCCTTGGTGCATCTTCCTGTTGAAAAGGAAAGCTGGATTGTGTC
TGGGACACAGTCTGGTACTCTCCTGGTCATCAATACCGAAGATGGGAAAAAGAGACATACCCTA
GAAAAGATGACTGATTCTGTCACTTGTTTGTATTGCAATTCCTTTTCCAAGCAAAGCAAACAAAAA
AATTTTCTTTTGGTTGGAACCGCTGATGGCAAGTTAGCAATTTTTGAAGATAAGACTGTTAAGCTT
AAAGGAGCTGCTCCTTTGAAGATACTAAATATAGGAAATGTCAGTACTCCATTGATGTGTTTGAGT
GAATCCACAAATTCAACGGAAAGAAATGTAATGTGGGGAGGATGTGGCACAAAGATTTTCTCCTT
TTCTAATGATTTCACCATTCAGAAACTCATTGAGACAAGAACAAGCCAACTGTTTTCTTATGCAGC
TTTCAGTGATTCCAACATCATAACAGTGGTGGTAGACACTGCTCTCTATATTGCTAAGCAAAATAG
CCCTGTTGTGGAAGTGTGGGATAAGAAAACTGAAAAACTCTGTGGACTAATAGACTGCGTGCACT
TTTTAAGGGAGGTAATGGTAAAAGAAAACAAGGAATCAAAACACAAAATGTCTTATTCTGGGAGA
GTGAAAACCCTCTGCCTTCAGAAGAACACTGCTCTTTGGATAGGAACTGGAGGAGGCCATATTTT
ACTCCTGGATCTTTCAACTCGTCGACTTATACGTGTAATTTACAACTTTTGTAATTCGGTCAGAGT
CATGATGACAGCACAGCTAGGAAGCCTTAAAAATGTCATGCTGGTATTGGGCTACAACCGGAAAA
ATACTGAAGGTACACAAAAGCAGAAAGAGATACAATCTTGCTTGACCGTTTGGGACATCAATCTT
CCACATGAAGTGCAAAATTTAGAAAAACACATTGAAGTGAGAAAAGAATTAGCTGAAAAAATGAG
ACGAACATCTGTTGAGTAA
Translated protein sequence for human G2019 Full length LRRK2 flag tagged protein
SEQ ID NO: 9
MDYKDDDDKMASGSCQGCEEDEETLKKLIVRLNNVQEGKQIETLVQILEDLLVFTYSEHASKLFQGKN
IHVPLLIVLDSYMRVASVQQVGWSLLCKLIEVCPGTMQSLMGPQDVGNDWEVLGVHQLILKMLTVHN
ASVNLSVIGLKTLDLLLTSGKITLLILDEESDIFMLIFDAMHSFPANDEVQKLGCKALHVLFERVSEEQLT
EFVENKDYMILLSALTNFKDEEEIVLHVLHCLHSLAIPCNNVEVLMSGNVRCYNIVVEAMKAFPMSERI
QEVSCCLLHRLTLGNFFNILVLNEVHEFVVKAVQQYPENAALQISALSCLALLTETIFLNQDLEEKNEN
QENDDEGEEDKLFWLEACYKALTWHRKNKHVQEAACWALNNLLMYQNSLHEKIGDEDGHFPAHRE
VMLSMLMHSSSKEVFQASANALSTLLEQNVNERKILLSKGIHLNVLELMQKHIHSPEVAESGCKMLNH
LFEGSNTSLDIMAAVVPKILTVMKRHETSLPVQLEALRAILHFIVPGMPEESREDTEFHHKLNMVKKQC
FKNDIHKLVLAALNRFIGNPGIQKCGLKVISSIVHFPDALEMLSLEGAMDSVLHTLQMYPDDQEIQCLG
LSLIGYLITKKNVFIGTGHLLAKILVSSLYRFKDVAEIQTKGFQTILAILKLSASFSKLLVHHSFDLVIFHQM
SSNIMEQKDQQFLNLCCKCFAKVAMDDYLKNVMLERACDQNNSIMVECLLLLGADANQAKEGSSLIC
QVCEKESSPKLVELLLNSGSREQDVRKALTISIGKGDSQIISLLLRRLALDVANNSICLGGFCIGKVEPS
WLGPLFPDKTSNLRKQTNIASTLARMVIRYQMKSAVEEGTASGSDGNFSEDVLSKFDEWTFIPDSSM
DSVFAQSDDLDSEGSEGSFLVKKKSNSISVGEFYRDAVLQRCSPNLQRHSNSLGPIFDHEDLLKRKR
KILSSDDSLRSSKLQSHMRHSDSISSLASEREYITSLDLSANELRDIDALSQKCCISVHLEHLEKLELHQ
NALTSFPQQLCETLKSLTHLDLHSNKFTSFPSYLLKMSCIANLDVSRNDIGPSVVLDPTVKCPTLKQFN
LSYNQLSEVPENLTDVVEKLEQLILEGNKISGICSPLRLKELKILNLSKNHISSLSENFLEACPKVESFSA
RMNFLAAMPFLPPSMTILKLSQNKFSCIPEAILNLPHLRSLDMSSNDIQYLPGPAHWKSLNLRELLFSH
NQISILDLSEKAYLWSRVEKLHLSHNKLKEIPPEIGCLENLTSLDVSYNLELRSFPNEMGKLSKIWDLPL
DELHLNFDFKHIGCKAKDIIRFLQQRLKKAVPYNRMKLMIVGNTGSGKTTLLQQLMKTKKSDLGMQSA
TVGIDVKDWPIQIRDKRKRDLVLNVWDFAGREEFYSTHPHFMTQRALYLAVYDLSKGQAEVDAMKP
WLFNIKARASSSPVILVGTHLDVSDEKQRKACMSKITKELLNKRGFPAIRDYHFVNATEESDALAKLRK
TIINESLNFKIRDQLVVGQLIPDCYVELEKIILSERKNVPIEFPVIDRKRLLQLVRENQLQLDENELPHAVH
FLNESGVLLHFQDPALQLSDLYFVEPKWLCKIMAQILTVKVEGCPKHPKGIISRRDVEKFLSKKRKFPK
NYMSQYFKLLEKFQIALPIGEEYLLVPSSLSDHRPVIELPHCENSEIIIRLYEMPYFPMGFWSRLINRLLE
ISPYMLSGRERALRPNRMYWRQGIYLNWSPEAYCLVGSEVLDNHPESELKITVPSCRKGCILLGQVV
DHIDSLMEEWFPGLLEIDICGEGETLLKKWALYSFNDGEEHQKILLDDLMKKAEEGDLLVNPDQPRLTI
PISQIAPDLILADLPRNIMLNNDELEFEQAPEFLLGDGSFGSVYRAAYEGEEVAVKIFNKHTSLRLLRQE
LVVLCHLHHPSLISLLAAGIRPRMLVMELASKGSLDRLLQQDKASLTRTLQHRIALHVADGLRYLHSAM
IIYRDLKPHNVLLFTLYPNAAIIAKIADYGIAQYCCRMGIKTSEGTPGFRAPEVARGNVIYNQQADVYSF
GLLLYDILTIGGRIVEGLKFPNEFDELEIQGKLPDPVKEYGCAPWPMVEKLIKQCLKENPQERPTSAQ
VFDILNSAELVCLTRRILLPKNVIVECMVATHHNSRNASIWLGCGHTDRGQLSFLDLNTEGYTSEEVAD
SRILCLALVHLPVEKESWIVSGTQSGTLLVINTEDGKKRHTLEKMTDSVTCLYCNSFSKQSKQKNFLLV
GTADGKLAIFEDKTVKLKGAAPLKILNIGNVSTPLMCLSESTNSTERNVMWGGCGTKIFSFSNDFTIQK
LIETRTSQLFSYAAFSDSNIITVVVDTALYIAKQNSPVVEVWDKKTEKLCGLIDCVHFLREVMVKENKES
KHKMSYSGRVKTLCLQKNTALWIGTGGGHILLLDLSTRRLIRVIYNFCNSVRVMMTAQLGSLKNVMLV
LGYNRKNTEGTQKQKEIQSCLTVWDINLPHEVQNLEKHIEVRKELAEKMRRTSVE
SEQ ID NO: 10: ′LRRKtide′ peptide
H-RLGRDKYKTLRQIRQ-OH

1-32. (canceled)

33. A compound of Formula (I): 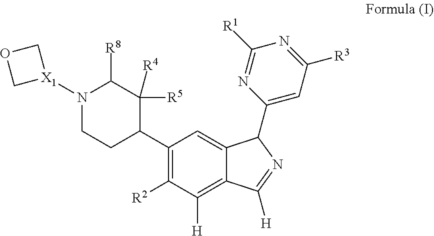
wherein:
R¹ is selected from the group consisting of CN, C₁₋₃ alkyl, C₁₋₃ alkoxy, C₁₋₃haloalkyl and C₃ cycloalkyl;
R² is selected from the group consisting of H, halo, CN, C₁₋₃alkyl and C₁₋₃haloalkyl;
R³ is selected from the group consisting of:
a) an N-linked 4-6 membered heterocyclyl ring optionally substituted with one, two, three or four substituents independently selected from the group consisting of halo, hydroxyl, C₁₋₆alkyl and C₁₋₆ alkoxyl;
wherein:
the C₁₋₆alkyl group optionally is substituted with one or two substituents independently selected from the group consisting of: halo, hydroxyl, C₁₋₃alkoxy and cyclopropyl, and
the C₁₋₆ alkoxyl group optionally is substituted with one or two substituents independently selected from the group consisting of halo, hydroxyl and C₁₋₃ alkoxyl,
when the N-linked 4-6 membered heterocyclyl ring contains a substitutable nitrogen atom, the N-linked 4-6 membered heterocyclyl ring optionally is substituted with a 4-6 membered heterocyclyl ring;
wherein:
the 4-6 membered heterocyclyl ring optionally is substituted with one, two or three substituents independently selected from halo, hydroxyl, and C₁₋₃ alkoxyl, and
the 4-6 membered heterocyclyl ring is attached to the substitutable nitrogen atom of the N-linked 4-6 membered heterocyclyl ring;
b) NHR⁷; and
c) OR⁷;
R⁴ and R⁵ are independently selected from the group consisting of H, hydroxyl and halo;
X₁ is CR⁶;
wherein:
R⁶ is C₁₋₃alkyl,
wherein:
the C₁₋₃alkyl group of R⁶ optionally is substituted with one or two substituents independently selected from the group consisting of hydroxyl, halo and C₁₋₃alkoxyl;
R⁷ is independently selected from the group consisting of C₄₋₆ cycloalkyl and a nitrogen- or oxygen-containing 4-6 membered heterocyclyl;
wherein:
the C₄₋₆ cycloalkyl group of R⁷ optionally is substituted with one, two or three substituents independently selected from halo, hydroxyl, C₁₋₃ alkoxyl and C₁₋₃ alkyl;
the nitrogen- or oxygen-containing 4-6 membered heterocyclyl of R⁷ optionally is substituted with one or more substituents independently selected from halo, hydroxyl, C₁₋₃ alkoxyl and C₁₋₃ alkyl;
the C₁₋₃ alkyl group defined for R⁷ and the nitrogen- or oxygen-containing 4-6 membered heterocyclyl defined for R⁷ optionally is substituted with one, two or three halo or hydroxyl groups; and
R⁸ is hydrogen or C₁₋₃alkyl; or
a pharmaceutically acceptable salt thereof.

34. A compound of Formula (I-A): 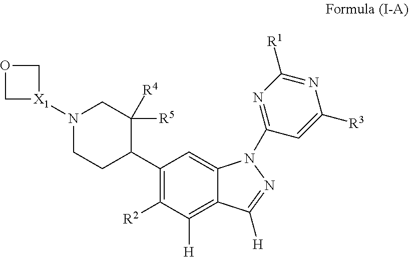
wherein:
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃haloalkyl;
R³ is selected from the group consisting of:
a) an N-linked 4-6 membered heterocyclyl ring optionally substituted with one or two substituents independently selected from the group consisting of: halo, hydroxyl, C₁₋₆alkyl and C₁₋₆ alkoxyl;
wherein:
the C₁₋₆alkyl optionally is substituted with one or two substituents independently selected from the group consisting of: halo, hydroxyl, C₁₋₃alkoxy and cyclopropyl,
the C₁₋₆ alkoxyl group is optionally substituted with one or two substituents independently selected from the group consisting of halo, hydroxyl and C₁₋₃ alkoxyl;
when the N-linked 4-6 membered heterocyclyl ring contains a substitutable nitrogen atom, the N-linked 4-6 membered heterocyclyl ring optionally is substituted with a 4-6 membered heterocyclyl ring;
wherein:
the 4-6 membered heterocyclyl ring optionally is substituted with one, two or three substituents independently selected from halo, hydroxyl, and C₁₋₃ alkoxyl; and
the 4-6 membered heterocyclyl ring is attached to the substitutable nitrogen atom of the N-linked 4-6 membered heterocyclyl ring;
b) NHR⁷; and
c) OR⁷;
R⁴ and R⁵ are independently selected from the group consisting of H, hydroxyl and halo;
X₁ is CR⁶;
wherein:
R⁶ is C₁₋₃alkyl is unsubstituted or optionally is substituted with one or two substituents independently selected from the group consisting of hydroxyl, halo and C₁₋₃alkoxyl;
R⁷ is independently selected from the group consisting of C₄₋₆ cycloalkyl and a nitrogen- or oxygen-containing 4-6 membered heterocyclyl;
wherein:
C₄₋₆ cycloalkyl, is optionally substituted with one, two or three substituents independently selected from halo, hydroxyl, C₁₋₃ alkoxyl and C₁₋₃ alkyl,
the C₁₋₃ alkyl group is optionally substituted with one two or three halo or hydroxyl groups, and
the nitrogen- or oxygen-containing 4-6 membered heterocyclyl optionally is substituted with one or more substituents independently selected from halo, hydroxyl, C₁₋₃ alkoxyl and C₁₋₃ alkyl,
wherein:
the C₁₋₃ alkyl group as defined for C₄₋₆ cycloalkyl and for the nitrogen- or oxygen-containing 4-6 membered heterocyclyl is optionally substituted with one two or three halo or hydroxyl groups; or
a pharmaceutically acceptable salt thereof

35. A compound of Formula (I-B): 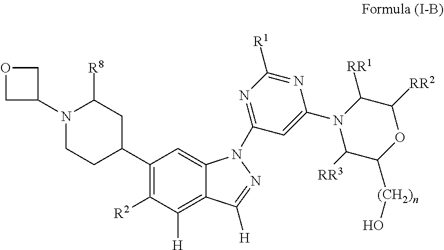
wherein:
R¹ is selected from the group consisting of CN, C₁₋₃ alkyl, C₁₋₃ alkoxy, C₁₋₃haloalkyl and C₃ cycloalkyl;
R² is selected from the group consisting of H, halo, CN, C₁₋₃alkyl and C₁₋₃haloalkyl;
RR¹, RR² and RR³ independently are hydrogen or C₁₋₃alkyl;
R⁸ is hydrogen or C₁₋₃alkyl;
n is 1 or 2; and
provided that:
when n is 1 and R⁸ is hydrogen, RR², RR¹ and RR³, respectively, are not all hydrogen; or
a pharmaceutically acceptable salt thereof:

36. A compound of Formula (I-C): 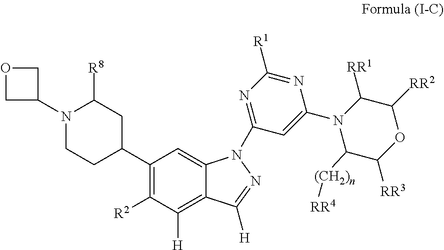
wherein:
R¹ is selected from the group consisting of CN, C₁₋₃ alkyl, C₁₋₃ alkoxy, C₁₋₃haloalkyl, and C₃ cycloalkyl;
R² is selected from the group consisting of H, halo, CN, C₁₋₃alkyl and C₁₋₃haloalkyl;
R⁸ is hydrogen or C₁₋₃alkyl;
RR¹, RR², and RR³ independently are hydrogen or C₁₋₃alkyl;
RR⁴ is hydrogen or hydroxyl;
n is 1 or 2; or
a pharmaceutically acceptable salt thereof

37. The compound of Formula (I) or a pharmaceutically acceptable salt thereof according to claim 33, wherein R¹ is selected from the group consisting of C₁₋₃ alkyl and C₁₋₃ alkoxyl.

38. The compound of Formula (I) or a pharmaceutically acceptable salt thereof according to claim 33, wherein R² is selected from the group consisting of H, halo and C₁₋₃alkyl.

39. The compound of Formula (I) or a pharmaceutically acceptable salt thereof according to claim 33, wherein R⁴ and R⁵ are independently selected from the group consisting of H and fluoro.

40. The compound of Formula (I) or a pharmaceutically acceptable salt thereof according to claim 33, wherein R⁴ and R⁵, respectively, are both H.

41. The compound of Formula (I) or a pharmaceutically acceptable salt thereof according to claim 33, wherein:
R³ is an N-linked 4-6 membered heterocyclyl ring optionally substituted with one or two substituents independently selected from the group consisting of halo, hydroxyl, C₁₋₃alkyl and C₁₋₃ alkoxyl;
wherein:
C₁₋₃alkyl group of R³ is optionally substituted with one or two substituents independently selected from the group consisting of: halo, hydroxyl and C₁₋₃alkoxy, and
C₁₋₃ alkoxyl group of R³ is optionally substituted with one or two substituents independently selected from halo, hydroxyl and C₁₋₃ alkoxyl.

42. The compound of Formula (I) or a pharmaceutically acceptable salt thereof, according to claim 33, wherein R⁶ is H or unsubstituted C₁₋₃alkyl.

43. The compound of Formula (I-B) or a pharmaceutically acceptable salt thereof according to claim 35, wherein n is 1, RR¹ is methyl, RR² is hydrogen, and RR³ is hydrogen.

44. The compound of Formula (I-B) or a pharmaceutically acceptable salt thereof according to claim 35, wherein n is 1, RR¹ is hydrogen, RR² is hydrogen, and RR³ is methyl.

45. The compound of Formula (I-B) or a pharmaceutically acceptable salt thereof according to claim 35, wherein n is 2, RR¹, RR² and RR³, respectively, are hydrogen.

46. The compound of Formula (I) or a pharmaceutically acceptable salt thereof according to claim 33, wherein R⁸ is selected from the group consisting of hydrogen and methyl.

47. The compound of Formula (I-C), or a pharmaceutically acceptable salt thereof according to claim 36, wherein n is 1.

48. The compound of Formula (I-C), or a pharmaceutically acceptable salt thereof according to claim 36, wherein RR² is hydrogen.

49. The compound of Formula (I-C), or a pharmaceutically acceptable salt thereof according to claim 36, wherein RR¹ is hydrogen.

50. The compound of Formula (I) or a pharmaceutically acceptable salt thereof according to claim 33, wherein R⁸ is hydrogen.

51. The compound of Formula (I-C) or a pharmaceutically acceptable salt thereof according to claim 36, wherein RR³ is hydrogen or methyl.

52. The compound of Formula (I-C) or a pharmaceutically acceptable salt thereof according to claim 36, wherein RR⁴ is hydrogen.

53. The compound of Formula (I-C) or a pharmaceutically acceptable salt thereof according to claim 36, wherein RR⁴ is hydroxyl.

54. A pharmaceutical composition comprising a compound of Formula (I) or a pharmaceutically acceptable salt thereof according to claim 33 and a pharmaceutically acceptable excipient.

55. A method for treating Parkinson's disease, which comprises administering to a subject in need thereof a therapeutically effective amount of a compound of Formula (I), or a pharmaceutically acceptable salt thereof according to claim 33.

56. The method for treating Parkinson's disease according to claim 55, wherein the subject is a human.

57. The method for treating Parkinson's disease according to claim 56, wherein the subject is a human expressing the G2019S mutation in the LRRK2 kinase.


SEQUENCE LISTING (10 sequences)

SEQ ID NO: 1 - DNA, 48 bp, Artificial
  Primers used for PCR cloning of Human G2019 LRRK2 plasmids preparation pHTBV-F
gatctcgacgggcgcggatccaccatggattacaaggatgacgacgat

SEQ ID NO: 2 - DNA, 49 bp, Artificial
  Primers used for PCR cloning of Human G2019 LRRK2 plasmids preparation LRRK2 wt-F1
catggattacaaggatgacgacgataagatggctagtggcagctgtcag

SEQ ID NO: 3 - DNA, 47 bp, Artificial
  Primers used for PCR cloning of Human G2019 LRRK2 plasmids preparation LRRK2 wt-R1
gttcacgagatccactattcagtaagagttccaccaatttgggactg

SEQ ID NO: 4 - DNA, 23 bp, Artificial
  Primers used for PCR cloning of Human G2019 LRRK2 plasmids preparation LRRK2 wt-F2
gaatagtggatctcgtgaacaag

SEQ ID NO: 5 - DNA, 25 bp, Artificial
  Primers used for PCR cloning of Human G2019 LRRK2 plasmids preparation LRRK2 wt-R2
gtcagacaaactgcttggaaccagc

SEQ ID NO: 6 - DNA, 40 bp, Artificial
  Primers used for PCR cloning of Human G2019 LRRK2 plasmids preparation LRRK2 wt-F3
ctggttccaagcagtttgtctgaccacaggcctgtgatag

SEQ ID NO: 7 - DNA, 46 bp, Artificial
  Primers used for PCR cloning of Human G2019 LRRK2 plasmids preparation pHTBV-R
gttctagccaagcttggtaccctattactcaacagatgttcgtctc

SEQ ID NO: 8 - DNA, 7611 bp, Artificial
  G2019  Full length Flag-LRRK2 coding sequence
atggattacaaggatgacgacgataagatggctagtggcagctgtcaggggtgcgaagaggacgaggaaactctgaagaagttgatagtcaggctgaacaatgtccaggaaggaaaacagatagaaacgctggtccaaatcctggaggatctgctggtgttcacgtactccgagcacgcctccaagttatttcaaggcaaaaatatccatgtgcctctgttgatcgtcttggactcctatatgagagtcgcgagtgtgcagcaggtgggttggtcacttctgtgcaaattaatagaagtctgtccaggtacaatgcaaagcttaatgggaccccaggatgttggaaatgattgggaagtccttggtgttcaccaattgattcttaaaatgctaacagttcataatgccagtgtaaacttgtcagtgattggactgaagaccttagatctcctcctaacttcaggtaaaatcaccttgctgatactggatgaagaaagtgatattttcatgttaatttttgatgccatgcactcatttccagccaatgatgaagtccagaaacttggatgcaaagctttacatgtgctgtttgagagagtctcagaggagcaactgactgaatttgttgagaacaaagattatatgatattgttaagtgcgttaacaaattttaaagatgaagaggaaattgtgcttcatgtgctgcattgtttacattccctagcgattccttgcaataatgtggaagtcctcatgagtggcaatgtcaggtgttataatattgtggtggaagctatgaaagcattccctatgagtgaaagaattcaagaagtgagttgctgtttgctccataggcttacattaggtaattttttcaatatcctggtattaaacgaagtccatgagtttgtggtgaaagctgtgcagcagtacccagagaatgcagcattgcagatctcagcgctcagctgtttggccctcctcactgagactattttcttaaatcaagatttagaggaaaagaatgagaatcaagagaatgatgatgagggggaagaagataaattgttttggctggaagcctgttacaaagcattaacgtggcatagaaagaacaagcacgtgcaggaggccgcatgctgggcactaaataatctccttatgtaccaaaacagtttacatgagaagattggagatgaagatggccatttcccagctcatagggaagtgatgctctccatgctgatgcattcttcatcaaaggaagttttccaggcatctgcgaatgcattgtcaactctcttagaacaaaatgttaatttcagaaaaatactgttatcaaaaggaatacacctgaatgttttggagttaatgcagaagcatatacattctcctgaagtggctgaaagtggctgtaaaatgctaaatcatctttttgaaggaagcaacacttccctggatataatggcagcagtggtccccaaaatactaacagttatgaaacgtcatgagacatcattaccagtgcagctggaggcgcttcgagctattttacattttatagtgcctggcatgccagaagaatccagggaggatacagaatttcatcataagctaaatatggttaaaaaacagtgtttcaagaatgatattcacaaactggtcctagcagctttgaacaggttcattggaaatcctgggattcagaaatgtggattaaaagtaatttcttctattgtacattttcctgatgcattagagatgttatccctggaaggtgctatggattcagtgcttcacacactgcagatgtatccagatgaccaagaaattcagtgtctgggtttaagtcttataggatacttgattacaaagaagaatgtgttcataggaactggacatctgctggcaaaaattctggtttccagcttataccgatttaaggatgttgctgaaatacagactaaaggatttcagacaatcttagcaatcctcaaattgtcagcatctttttctaagctgctggtgcatcattcatttgacttagtaatattccatcaaatgtcttccaatatcatggaacaaaaggatcaacagtttctaaacctctgttgcaagtgttttgcaaaagtagctatggatgattacttaaaaaatgtgatgctagagagagcgtgtgatcagaataacagcatcatggttgaatgcttgcttctattgggagcagatgccaatcaagcaaaggagggatcttctttaatttgtcaggtatgtgagaaagagagcagtcccaaattggtggaactcttactgaatagtggatctcgtgaacaagatgtacgaaaagcgttgacgataagcattgggaaaggtgacagccagatcatcagcttgctcttaaggaggctggccctggatgtggccaacaatagcatttgccttggaggattttgtataggaaaagttgaaccttcttggcttggtcctttatttccagataagacttctaatttaaggaaacaaacaaatatagcatctacactagcaagaatggtgatcagatatcagatgaaaagtgctgtggaagaaggaacagcctcaggcagcgatggaaatttttctgaagatgtgctgtctaaatttgatgaatggacctttattcctgactcttctatggacagtgtgtttgctcaaagtgatgacctggatagtgaaggaagtgaaggctcatttcttgtgaaaaagaaatctaattcaattagtgtaggagaattttaccgagatgccgtattacagcgttgctcaccaaatttgcaaagacattccaattccttggggcccatttttgatcatgaagatttactgaagcgaaaaagaaaaatattatcttcagatgattcactcaggtcatcaaaacttcaatcccatatgaggcattcagacagcatttcttctctggcttctgagagagaatatattacatcactagacctttcagcaaatgaactaagagatattgatgccctaagccagaaatgctgtataagtgttcatttggagcatcttgaaaagctggagcttcaccagaatgcactcacgagctttccacaacagctatgtgaaactctgaagagtttgacacatttggacttgcacagtaataaatttacatcatttccttcttatttgttgaaaatgagttgtattgctaatcttgatgtctctcgaaatgacattggaccctcagtggttttagatcctacagtgaaatgtccaactctgaaacagtttaacctgtcatataaccagctgtcttttgtacctgagaacctcactgatgtggtagagaaactggagcagctcattttagaaggaaataaaatatcagggatatgctcccccttgagactgaaggaactgaagattttaaaccttagtaagaaccacatttcatccctatcagagaactttcttgaggcttgtcctaaagtggagagtttcagtgccagaatgaattttcttgctgctatgcctttcttgcctccttctatgacaatcctaaaattatctcagaacaaattttcctgtattccagaagcaattttaaatcttccacacttgcggtctttagatatgagcagcaatgatattcagtacctaccaggtcccgcacactggaaatctttgaacttaagggaactcttatttagccataatcagatcagcatcttggacttgagtgaaaaagcatatttatggtctagagtagagaaactgcatctttctcacaataaactgaaagagattcctcctgagattggctgtcttgaaaatctgacatctctggatgtcagttacaacttggaactaagatcctttcccaatgaaatggggaaattaagcaaaatatgggatcttcctttggatgaactgcatcttaactttgattttaaacatataggatgtaaagccaaagacatcataaggtttcttcaacagcgattaaaaaaggctgtgccttataaccgaatgaaacttatgattgtgggaaatactgggagtggtaaaaccaccttattgcagcaattaatgaaaaccaagaaatcagatcttggaatgcaaagtgccacagttggcatagatgtgaaagactggcctatccaaataagagacaaaagaaagagagatctcgtcctaaatgtgtgggattttgcaggtcgtgaggaattctatagtactcatccccattttatgacgcagcgagcattgtaccttgctgtctatgacctcagcaagggacaggctgaagttgatgccatgaagccttggctcttcaatataaaggctcgcgcttcttcttcccctgtgattctcgttggcacacatttggatgtttctgatgagaagcaacgcaaagcctgcatgagtaaaatcaccaaggaactcctgaataagcgagggttccctgccatacgagattaccactttgtgaatgccaccgaggaatctgatgctttggcaaaacttcggaaaaccatcataaacgagagccttaatttcaagatccgagatcagcttgttgttggacagctgattccagactgctatgtagaacttgaaaaaatcattttatcggagcgtaaaaatgtgccaattgaatttcccgtaattgaccggaaacgattattacaactagtgagagaaaatcagctgcagttagatgaaaatgagcttcctcacgcagttcactttctaaatgaatcaggagtccttcttcattttcaagacccagcactgcagttaagtgacttgtactttgtggaacccaagtggctttgtaaaatcatggcacagattttgacagtgaaagtggaaggttgtccaaaacaccctaagggaattatttcgcgtagagatgtggaaaaatttctttcaaagaaaaggaaatttccaaagaactacatgtcacagtattttaagctcctagaaaaattccagattgctttgccaataggagaagaatatttgctggttccaagcagtttgtctgaccacaggcctgtgatagagcttccccattgtgagaactctgaaattatcatccgactatatgaaatgccttattttccaatgggattttggtcaagattaatcaatcgattacttgagatttcaccttacatgctttcagggagagaacgagcacttcgcccaaacagaatgtattggcgacaaggcatttacttaaattggtctcctgaagcttattgtctggtaggatctgaagtcttagacaatcatccagagagtttcttaaaaattacagttccttcttgtagaaaaggctgtattcttttgggccaagttgtggaccacattgattctctcatggaagaatggtttcctgggttgctggagattgatatttgtggtgaaggagaaactctgttgaagaaatgggcattatatagttttaatgatggtgaagaacatcaaaaaatcttacttgatgacttgatgaagaaagcagaggaaggagatctcttagtaaatccagatcaaccaaggctcaccattccaatatctcagattgcccctgacttgattttggctgacctgcctagaaatattatgttgaataatgatgagttggaatttgaacaagctccagagtttctcctaggtgatggcagttttggatcagtttaccgagcagcctatgaaggagaagaagtggctgtgaagatttttaataaacatacatcactcaggctgttaagacaagagcttgtggtgctttgccacctccaccaccccagtttgatatctttgctggcagctgggattcgtccccggatgttggtgatggagttagcctccaagggttccttggatcgcctgcttcagcaggacaaagccagcctcactagaaccctacagcacaggattgcactccacgtagctgatggtttgagatacctccactcagccatgattatataccgagacctgaaaccccacaatgtgctgcttttcacactgtatcccaatgctgccatcattgcaaagattgctgactacggcattgctcagtactgctgtagaatggggataaaaacatcagagggcacaccagggtttcgtgcacctgaagttgccagaggaaatgtcatttataaccaacaggctgatgtttattcatttggtttactactctatgacattttgacaactggaggtagaatagtagagggtttgaagtttccaaatgagtttgatgaattagaaatacaaggaaaattacctgatccagttaaagaatatggttgtgccccatggcctatggttgagaaattaattaaacagtgtttgaaagaaaatcctcaagaaaggcctacttctgcccaggtctttgacattttgaattcagctgaattagtctgtctgacgagacgcattttattacctaaaaacgtaattgttgaatgcatggttgctacacatcacaacagcaggaatgcaagcatttggctgggctgtgggcacaccgacagaggacagctctcatttcttgacttaaatactgaaggatacacttctgaggaagttgctgatagtagaatattgtgcttagccttggtgcatcttcctgttgaaaaggaaagctggattgtgtctgggacacagtctggtactctcctggtcatcaataccgaagatgggaaaaagagacataccctagaaaagatgactgattctgtcacttgtttgtattgcaattccttttccaagcaaagcaaacaaaaaaattttcttttggttggaaccgctgatggcaagttagcaatttttgaagataagactgttaagcttaaaggagctgctcctttgaagatactaaatataggaaatgtcagtactccattgatgtgtttgagtgaatccacaaattcaacggaaagaaatgtaatgtggggaggatgtggcacaaagattttctccttttctaatgatttcaccattcagaaactcattgagacaagaacaagccaactgttttcttatgcagctttcagtgattccaacatcataacagtggtggtagacactgctctctatattgctaagcaaaatagccctgttgtggaagtgtgggataagaaaactgaaaaactctgtggactaatagactgcgtgcactttttaagggaggtaatggtaaaagaaaacaaggaatcaaaacacaaaatgtcttattctgggagagtgaaaaccctctgccttcagaagaacactgctctttggataggaactggaggaggccatattttactcctggatctttcaactcgtcgacttatacgtgtaatttacaacttttgtaattcggtcagagtcatgatgacagcacagctaggaagccttaaaaatgtcatgctggtattgggctacaaccggaaaaatactgaaggtacacaaaagcagaaagagatacaatcttgcttgaccgtttgggacatcaatcttccacatgaagtgcaaaatttagaaaaacacattgaagtgagaaaagaattagctgaaaaaatgagacgaacatctgttgagtaa

SEQ ID NO: 9 - protein, 2536 aa, Artificial
  Translated protein sequence for human G2019 Full length LRRK2 flag tagged protein
MDYKDDDDKMASGSCQGCEEDEETLKKLIVRLNNVQEGKQIETLVQILEDLLVFTYSEHASKLFQGKNIHVPLLIVLDSYMRVASVQQVGWSLLCKLIEVCPGTMQSLMGPQDVGNDWEVLGVHQLILKMLTVHNASVNLSVIGLKTLDLLLTSGKITLLILDEESDIFMLIFDAMHSFPANDEVQKLGCKALHVLFERVSEEQLTEFVENKDYMILLSALTNFKDEEEIVLHVLHCLHSLAIPCNNVEVLMSGNVRCYNIVVEAMKAFPMSERIQEVSCCLLHRLTLGNFFNILVLNEVHEFVVKAVQQYPENAALQISALSCLALLTETIFLNQDLEEKNENQENDDEGEEDKLFWLEACYKALTWHRKNKHVQEAACWALNNLLMYQNSLHEKIGDEDGHFPAHREVMLSMLMHSSSKEVFQASANALSTLLEQNVNFRKILLSKGIHLNVLELMQKHIHSPEVAESGCKMLNHLFEGSNTSLDIMAAVVPKILTVMKRHETSLPVQLEALRAILHFIVPGMPEESREDTEFHHKLNMVKKQCFKNDIHKLVLAALNRFIGNPGIQKCGLKVISSIVHFPDALEMLSLEGAMDSVLHTLQMYPDDQEIQCLGLSLIGYLITKKNVFIGTGHLLAKILVSSLYRFKDVAEIQTKGFQTILAILKLSASFSKLLVHHSFDLVIFHQMSSNIMEQKDQQFLNLCCKCFAKVAMDDYLKNVMLERACDQNNSIMVECLLLLGADANQAKEGSSLICQVCEKESSPKLVELLLNSGSREQDVRKALTISIGKGDSQIISLLLRRLALDVANNSICLGGFCIGKVEPSWLGPLFPDKTSNLRKQTNIASTLARMVIRYQMKSAVEEGTASGSDGNFSEDVLSKFDEWTFIPDSSMDSVFAQSDDLDSEGSEGSFLVKKKSNSISVGEFYRDAVLQRCSPNLQRHSNSLGPIFDHEDLLKRKRKILSSDDSLRSSKLQSHMRHSDSISSLASEREYITSLDLSANELRDIDALSQKCCISVHLEHLEKLELHQNALTSFPQQLCETLKSLTHLDLHSNKFTSFPSYLLKMSCIANLDVSRNDIGPSVVLDPTVKCPTLKQFNLSYNQLSFVPENLTDVVEKLEQLILEGNKISGICSPLRLKELKILNLSKNHISSLSENFLEACPKVESFSARMNFLAAMPFLPPSMTILKLSQNKFSCIPEAILNLPHLRSLDMSSNDIQYLPGPAHWKSLNLRELLFSHNQISILDLSEKAYLWSRVEKLHLSHNKLKEIPPEIGCLENLTSLDVSYNLELRSFPNEMGKLSKIWDLPLDELHLNFDFKHIGCKAKDIIRFLQQRLKKAVPYNRMKLMIVGNTGSGKTTLLQQLMKTKKSDLGMQSATVGIDVKDWPIQIRDKRKRDLVLNVWDFAGREEFYSTHPHFMTQRALYLAVYDLSKGQAEVDAMKPWLFNIKARASSSPVILVGTHLDVSDEKQRKACMSKITKELLNKRGFPAIRDYHFVNATEESDALAKLRKTIINESLNFKIRDQLVVGQLIPDCYVELEKIILSERKNVPIEFPVIDRKRLLQLVRENQLQLDENELPHAVHFLNESGVLLHFQDPALQLSDLYFVEPKWLCKIMAQILTVKVEGCPKHPKGIISRRDVEKFLSKKRKFPKNYMSQYFKLLEKFQIALPIGEEYLLVPSSLSDHRPVIELPHCENSEIIIRLYEMPYFPMGFWSRLINRLLEISPYMLSGRERALRPNRMYWRQGIYLNWSPEAYCLVGSEVLDNHPESFLKITVPSCRKGCILLGQVVDHIDSLMEEWFPGLLEIDICGEGETLLKKWALYSFNDGEEHQKILLDDLMKKAEEGDLLVNPDQPRLTIPISQIAPDLILADLPRNIMLNNDELEFEQAPEFLLGDGSFGSVYRAAYEGEEVAVKIFNKHTSLRLLRQELVVLCHLHHPSLISLLAAGIRPRMLVMELASKGSLDRLLQQDKASLTRTLQHRIALHVADGLRYLHSAMIIYRDLKPHNVLLFTLYPNAAIIAKIADYGIAQYCCRMGIKTSEGTPGFRAPEVARGNVIYNQQADVYSFGLLLYDILTTGGRIVEGLKFPNEFDELEIQGKLPDPVKEYGCAPWPMVEKLIKQCLKENPQERPTSAQVFDILNSAELVCLTRRILLPKNVIVECMVATHHNSRNASIWLGCGHTDRGQLSFLDLNTEGYTSEEVADSRILCLALVHLPVEKESWIVSGTQSGTLLVINTEDGKKRHTLEKMTDSVTCLYCNSFSKQSKQKNFLLVGTADGKLAIFEDKTVKLKGAAPLKILNIGNVSTPLMCLSESTNSTERNVMWGGCGTKIFSFSNDFTIQKLIETRTSQLFSYAAFSDSNIITVVVDTALYIAKQNSPVVEVWDKKTEKLCGLIDCVHFLREVMVKENKESKHKMSYSGRVKTLCLQKNTALWIGTGGGHILLLDLSTRRLIRVIYNFCNSVRVMMTAQLGSLKNVMLVLGYNRKNTEGTQKQKEIQSCLTVWDINLPHEVQNLEKHIEVRKELAEKMRRTSVE

SEQ ID NO: 10 - protein, 15 aa, Artificial
  ′LRRKtide′ peptide
RLGRDKYKTLRQIRQ